(19) United States
(12) Patent Application Publication
(10) Pub. No.: US 2014/0024548 A1
(43) Pub. Date: Jan. 23, 2014

(54) DRUG SELECTION FOR MALIGNANT CANCER THERAPY USING ANTIBODY-BASED ARRAYS

(71) Applicant: NESTEC S.A., Vevey (CH)

(72) Inventors: Sharat Singh, Rancho Santa Fe, CA (US); Nicholas Hoe, San Diego, CA (US); Fred Princen, La Jolla, CA (US); Xinjun Liu, San Diego, CA (US)

(21) Appl. No.: 13/923,316

(22) Filed: Jun. 20, 2013

Related U.S. Application Data
(63) Continuation of application No. PCT/US2011/066624, filed on Dec. 21, 2011.
(60) Provisional application No. 61/438,904, filed on Feb. 2, 2011.
(60) Provisional application No. 61/426,948, filed on Dec. 23, 2010.

(51) Int. Cl. G01N 33/574

(52) U.S. Cl. CPC G01N 33/5748
USPC 506/9

(57) ABSTRACT

The present invention provides methods for selecting a suitable anticancer drug therapy, and for identifying and predicting response, for the treatment of a malignant cancer involving aberrant c-Met signaling. The present invention also provides methods for monitoring the status of a malignant cancer involving aberrant cMet signaling and monitoring how a patient with the malignant cancer is responding to anticancer drug therapy.

### CROSS-REFERENCE TO RELATED APPLICATIONS

The present application is a continuation of PCT/2011/066624, filed Dec. 21, 2011, which application claims priority to U.S. Provisional Application No. 61/438,904, filed Feb. 2, 2011, and U.S. Provisional Application No. 61/426,948, filed Dec. 23, 2010, the disclosures of which are herein incorporated by reference in their entirety for all purposes.

### BACKGROUND OF THE INVENTION

A wide variety of human malignancies are caused by sustained cMet stimulation, overexpression, or mutation, including carcinomas of the breast, liver, lung, ovary, kidney, and thyroid. Activation of cMet can induce increases in cell growth, invasion, angiogenesis and metastasis. For example, activating mutations in the cMet gene (MET) have been identified in patients with a particular hereditary form of papillary renal cancer, directly implicating cMet in human tumorigenesis. Dysregulation of cMET signaling due to activation of the cMet receptor via genomic alterations or increases in protein expression has been associated with an array of primary tumors. For instance, 41-72% of lung tumors from patients with primary tumors exhibited increased cMet expression, and 8-13% of the tumors carried MET mutations.

cMet is a tyrosine kinase receptor that is mutated and overexpressed in many cancers, in particular non-small cell lung cancer. Upon binding to its ligand HGF, cMet dimerizes and autophosphorylates tyrosine residues in its kinase domain. This induces recruitment of adaptor proteins and signal transduction through numerous downstream cascades such as the MAPK and AKT pathways.

Aberrant cMet signaling is implicated in cancer progression, as well as in a patient's response to anticancer drugs. Amplification of the cMET gene which results in increased protein expression, as well as HGF stimulation both can induce drug resistance in lung cancer cells by activating the PI3K-AKT pathway (see, e.g., McDermott et al. Cancer Res., 71:1625-1634 (2010); Engelman et al. Science, 316:1039-1043, (2007); and Yano et al., Cancer Res., 68:9479-9487 (2008)). Engelman et al. also shows that in the presence of a tyrosine kinase inhibitor (gefitinib) the PI3K-AKT pathway is activated via phosphorylation of HER3 by cMet. Zucali et al. (Ann. Oncol., 19:1605-12 (2008)) describes that elevated expression of activated cMet significantly correlates to a shorter time to progression (TTP) in patients with non-small cell lung cancer (NSCLC).

The implication of cMet signaling in tumor growth and progression has led to the development of a variety of anticancer drugs aimed at blocking cMet signaling. Holmes et al. (J. Mol. Biol., 367:395-408, (2007)) describes that the N-terminus of HGF can bind, but not activate cMet signaling, suggesting that this may be an effective method of antagonizing the cMet pathway. Examples of current c-Met drugs under development include neutralizing antibodies such as MAG102 (Amgen) and MetMab (Roche), and tyrosine kinase inhibitors (TKIs) such as ARQ 197, XL 184, PF-02341066, GSK1363089/XL880, INC280, MP470, MGCD265, SGX523, PF04217903 and JNJ38877605. Preliminary clinical results of several of these drug agents have been encouraging.

Tyrosine kinase inhibitors directed to epidermal growth factor receptor (EGFR), such as gefitinib and erlotinib, have been used to treat cancers including NSCLC. These drugs have been shown to elicit partial responses in 10-20% of NSCLC patients (Fukuoko et al. J. Clin. Oncol, 21:2237-2246 (2003) and Kris et al. JAMA, 290:2149-2158 (2003)). Of those NSCLC patients harboring EGFR mutations and on EGFR-TKI therapy, 70-75% show a positive response rate (see, e.g. Yano et al., Cancer Res., 68:9479-9487 (2008)). However, 25-30% of the patients are intrinsically resistant to EGFR-TKIs. Moreover, even those patients who are initial responders to treatment acquire resistance with time. Cappuzzo et al. (J. Clin. Oncol., 27:1667-1674 (2009)) describes that tumor samples from surgically resected NSCLC patients exhibited increased MET copy number as assayed by fluorescent in situ hybridization (FISH). A subset (20%) of tumor samples from surgically resected NSCLC patients exhibited increased copy numbers of EGFR and MET, suggesting a correlation between EGFR- and cMet-TKI resistance (Cappuzzo et al., J. Clin. Oncol., 27:1667-1674 (2009)). Strikingly, McDermott et al. shows that sensitivity to EGFR TKIs is associated with acquired resistance to cMet TKIs.

Thus, there is a need in the art for assays to detect the presence of aberrant cMet signaling in a patient sample to monitor cMet inhibitor therapy and to guide treatment decisions. The present invention satisfies this need and provides related advantages as well.

### BRIEF SUMMARY OF THE INVENTION

The present invention provides compositions and methods for detecting the status (e.g., expression and/or activation levels) of components of signal transduction pathways in tumor cells (e.g., non-small cell lung cancer cells). Information on the expression and/or activation states of components of signal transduction pathways (e.g., HER3 and/or c-Met signal transduction pathway components) derived from practice of the present invention can be used for malignant cancer diagnosis, prognosis, and in the design of cancer treatments for malignancies involving aberrant c-Met signaling.

In one aspect, the present invention provides a method for therapy selection for a subject with a malignancy involving aberrant c-Met signaling, the method comprising:
(a) detecting and/or quantifying the expression level and/or activation level of cMet protein in a sample taken from the subject;
(b) detecting and/or quantifying the expression level and/or activation level of HER3 protein in the sample;
(c) comparing the expression level and/or activation level of cMet protein and/or HER3 protein in the sample to (i) the expression level and/or activation level of a control protein and/or (ii) the expression level and/or activation level of cMet protein and/or HER3 protein in a control sample; and
(d) determining whether to administer a cMet inhibitor alone or a cMet inhibitor in combination with a pathway-directed therapy based upon a difference between the expression level and/or activation level of cMet protein and/or HER3 protein in the sample compared to the control protein and/or control sample.

In certain embodiments, step (d) comprises administering a cMet inhibitor alone or a cMet inhibitor in combination with a pathway-directed therapy based upon the differences between the expression and/or activation levels of cMet protein and/or HER3 protein in the sample compared to the control protein and/or control sample. In certain instances, the level of expression or activation of the control protein corresponds to a cut-off or threshold value. In other instances, the level of expression or activation of cMet protein or HER3 protein in the control sample corresponds to a cut-off or threshold value.

In some embodiments, the methods of the present invention may be useful to aid or assist in the selection of a suitable anticancer drug (e.g., cMet inhibitor) for the treatment of a malignancy involving aberrant c-Met signaling. In other embodiments, the methods of the present invention may be useful for improving the selection of a suitable anticancer drug (e.g., cMet inhibitor) for the treatment of a malignancy involving aberrant c-Met signaling. In yet other embodiments, the methods of the present invention may be useful to predict or identify the response (or likelihood of response) of a malignancy involving aberrant c-Met signaling or the response (or likelihood of response) of a subject having the malignancy to treatment with an anticancer drug (e.g., cMet inhibitor).

In another aspect, the present invention provides a method for monitoring the status of a malignancy involving aberrant cMet signaling in a subject or monitoring how a patient with the malignancy is responding to a therapy, the method comprising:
(a) detecting and/or quantifying serial changes to the expression level and/or activation level of cMet protein in a sample taken from the subject;
(b) detecting and/or quantifying serial changes to the expression level and/or activation level of HER3 protein in the sample; and
(c) comparing the expression level and/or activation level of cMet protein and/or HER3 protein in the sample to (i) the expression level and/or activation level of a control protein over time and/or (ii) the expression level and/or activation level of cMet protein and/or HER3 protein in a control sample over time,
wherein an increasing expression level and/or activation level of cMet protein and/or HER3 protein over time indicates disease progression or a negative response to the therapy, and
wherein a decreasing expression level and/or activation level of cMet protein and/or HER3 protein over time indicates disease remission or a positive response to the therapy.

In some embodiments, the methods of the present invention may be useful to aid or assist in monitoring the status of a malignancy involving aberrant cMet signaling in a subject or monitoring how a patient with the malignancy is responding to anticancer drug (e.g., cMet inhibitor) therapy. In other embodiments, the methods of the present invention may be useful for improving the monitoring of the status of a malignancy involving aberrant cMet signaling in a subject or monitoring of how a patient with the malignancy is responding to anticancer drug (e.g., cMet inhibitor) therapy.

The disclosures of the following patent documents are herein incorporated by reference in their entirety for all purposes: PCT Publication No. WO 2008/036802; PCT Publication No. WO 2009/012140; PCT Publication No. WO 2009/108637; PCT Publication No. WO 2010/132723; PCT Publication No. WO 2011/008990; and PCT Publication No. WO 2011/050069.

Other objects, features, and advantages of the present invention will be apparent to one of skill in the art from the following detailed description and figures.

### BRIEF DESCRIPTION OF THE DRAWINGS

FIG. 1 shows the expression profiling of HER1, HER2, HER3, cMet, IGF1R, cKit, PI3K, and Shc in NSCLC tumor tissue samples from Caucasian patients using the Collaborative Enzyme Enhanced Reactive ImmunoAssay (CEER) described herein. IgG and cytokeratin (CK) were used as controls.

FIGS. 2A-2D show the expression levels of total HER1 and HER2 proteins in NSCLC tumor tissue samples from both Asian patients and Caucasian patients as determined by CEER.

FIGS. 3A-3D show the expression levels of total HER3 and cMET proteins in NSCLC tumor tissue samples from both Asian patients and Caucasian patients as determined by CEER.

FIG. 4A shows a summary table of the expression levels of HER1, HER2. HER3, cMET and CK in NSCLC tumor tissue samples from Asian patients. FIG. 4B shows a summary table of the expression levels of HER1, HER2. HER3, cMET and CK in NSCLC tumor tissue samples from Caucasian patients.

FIG. 5 shows a comparison of the differential HER3 and cMET profiling of NSCLC tumor tissue samples from Asian and Caucasian patients.

FIG. 6 shows another embodiment of the invention, which is particularly useful in determining activated (e.g., phosphorylated) and total analyte levels in a biological sample. As a non-limiting example, expression profiles of total HER3, cMET and PI3K, as well as phosphorylated HER3, cMET and PI3K can be detected using the CEER array.

FIG. 7 shows the expression profiling of HER1, HER2, HER3, c-Met, IGF1R, cKit, PI3K, and Shc in NSCLC tumor tissue samples from Asian patients using CEER. IgG and cytokeratin (CK) were used as controls.

FIG. 8 shows the expression profiling of VEGFR2 in NSCLC tumor tissue samples from Asian patients using CEER. IgG was used as a control.

FIGS. 9A-9D show the expression of activated cMET, HER2, HER3 and PI3K in N87 cells treated with various dosages of cMET inhibitor as determined by CEER.

FIGS. 10A-10E show the expression profiles using CEER of HER1, HER2, HER3, c-Met, IGF1R, cKit, PI3K, and Shc protein in HCC827 cells treated with varying amounts HGF. IgG and CK were used as controls. In a non-limiting example, a range of HGF was used to treat the cells.

### DETAILED DESCRIPTION OF THE INVENTION

### I. Introduction

cMet is a tyrosine kinase receptor consisting of a 50-kDa extracellular alpha-chain and a 140-kDa transmembrane beta-chain linked by disulfide bonds. Upon binding to hepatocyte growth factor/scatter factor (HGF/SF), its ligand, cMet dimerizes and autophosphorylates tyrosine residues in its kinase domain. This induces recruitment of adaptor proteins and signal transduction through numerous downstream signaling cascades such as the MAPK and AKT pathways. Together, HGF/SF and cMet comprise a well-characterized ligand/receptor complex involved in both multiple cellular signaling pathways and numerous cellular functions, including proliferation, cell survival, motility and morphogenesis.

Dysregulation of cMet signaling has been implicated in many different types of cancer, including colon, gastric, bladder, breast, ovarian, pancreatic, kidney, liver, lung, head and neck, thyroid, and pancreatic cancers (see, e.g., Peruzzi, B and Bottaro, D P, Clin. Cancer Res., 12:3657-3660 (2006). In tumor cells, activation of cMet signaling triggers a diverse series of signaling cascades (e.g., MAPK, PI3K, VEGFR, EGFR, HER2 and HER3 pathways) resulting in cell growth, proliferation, invasion, migration, protection from apoptosis, and metastasis (see, e.g., Eder et al., Clin. Cancer Res., 15: 2207-2214 (2009)). For example, cMet signaling induces tumor angiogenesis by inducing proliferation and migration of endothelial cells, as well as inducing the expression of VEGF (see, e.g., Rosen et al., Adv. Cancer Res., 67: 257-279 (1995)). cMet has been demonstrated to interact with and phosphorylate kinases such as RON, EGFR, HER2, HER3, PI3K, and SHC. cMet may interact with other kinases as well, e.g., p95HER2, IGF-1R, c-KIT, and others. Aberrant signaling of the cMet signaling pathway due to dysregulation of the cMet receptor or overexpression of its ligand, hepatocyte growth factor/scatter factor (HGF/SF), has been associated with an aggressive papillary renal cancer.

Aberrant cMet signaling can be due to genomic alteration such as gene amplication or mutation, as well as overexpression of cMet protein and HGF/SF. Gene amplification and/or activating mutations in the cMet gene have been identified within the tyrosine kinase, juxtamembrane, and semaphorin domains of the receptor. Analysis of patients with primary tumors revealed that 41-72% of patients with primary lung tumors have tumor cells that overexpress cMet protein, while 8-13% of the patient have lung tumor cells that carry cMet mutations (see, Table 3 in Example 4). It has been shown that mutated and overexpressed cMet is typically associated with a worse prognosis for cancer. More importantly, within the last few years, aberrant cMet signaling has been correlated to resistance to anticancer therapies (e.g., EGFR inhibitor, specifically EGFR tyrosine kinase inhibitors (EGFR TKIs)).

A number of therapeutic strategies have been employed to inhibit cMet, such as monoclonal antibodies and small-molecule tyrosine kinase inhibitors. Examples of cMet inhibitors under development include neutralizing antibodies, such as MAG102 (Amgen) and MetMab (Roche), as well as tyrosine kinase inhibitors (TKIs), such as ARQ 197, XL 184, PF-02341066, GSK1363089/XL880, MP470, MGCD265, SGX523, PF04217903 and JNJ38877605. It has been demonstrated that cMET and HGF/SF are markers of positive response to cMET inhibitors (see, ARQ127 Study in MetMab, European Society for Medical Oncology Congress, Oct. 17, 2010). Yet interestingly, patients with a malignancy (e.g., gastric cancer) and cMet gene amplification do not respond to tyrosine kinase inhibitors. Engelman et al. (Science, 316:1039-1043, (2007)) demonstrated that resistance to the EGFR TKI gefitinib is associated with activated cMet which activates HER3 and the PI3K-AKT signaling pathway. Resistance to cMET and/or EGFR inhibitors may be attributed to functional redundancies among multiple signaling pathways. Thus, in many cases, there is a need to employ multi-targeted therapies to overcome resistance to tyrosine kinase inhibitors and to effectively treat malignancies involving aberrant cMet signaling.

It has been shown that treatment of gastric cancer cells overexpressing HER2 (e.g., N87 cells) with a cMet inhibitor caused an increased level of activated cMet, EGFR, HER2, and HER3 proteins. Treatment of NSCLC cells (e.g., HCC827 cells) with a cMet inhibitor induced activation of cMet and PI3K via activation of the HER2-HER3 and PI3K-AKT signaling pathway (see, Example 10).

A Phase II clinical study in Caucasian patients with NSCLC demonstrated that positive response to cMet inhibitors (see, ARQ197 Study in MetMab; European Society for Medical Oncology Congress, Oct. 17, 2010) can lead to an increase in both progression-free survival and survival. In an exemplary demonstration of the present invention, most Caucasian patients with NSCLC exhibited low expression of HER1 and HER2, and more than 50% of said patients also had high expression of cMet and HER3 (see, Example 8). Further analysis revealed that Caucasian patients with positive responses to cMet inhibitor therapy also expressed the activated forms of cMet and HER3 (phospho-cMet and phospho-HER3, respectively), indicating that the cMet and HER3 signaling pathways can be co-activated in tumor cells from these patients. Possible mechanisms for the co-activation include interactions of cMet with truncated HER3 protein or interaction of HER3 with truncated cMet protein. Henceforth, cMet and HER3 are markers of positive response to cMET inhibitor therapy in Caucasian patients with NSCLC. In another demonstration of the present invention, it was shown that Caucasian patients with NSCLC with KRAS mutations exhibited high levels of cMet and HER3 expression (see, Example 8). In addition, it was predicted using the present invention that treating these Caucasian patients with a cMet inhibitor alone such as monotherapy would result in a positive response due to inhibition of both the cMet and HER3-PI3K pathways.

In another exemplary demonstration of the present invention, samples obtained from Asian patients with NSCLC exhibited high levels of HER1 concomitant with expression of activated (i.e., phosphorylated) HER1 (see, Example 9). Unlike in the Caucasian patients, the Asian patients did not express high levels of cMet and HER3 (see, Example 9). Based on the predictive method of the present invention, cMet inhibitors are not expected to be effective in Asian patients with NSCLC. The expression/activation profile of Asian patients with NSCLC correlates with the data supporting the notion that EGFR inhibitors are highly effective in Asian patients.

The present invention provides methods for therapy selection for a patient with a malignancy involving aberrant cMet signaling based on detecting, quantifying, and comparing the activity of particular signal transduction pathways, and components thereof, which serve as expression/activation profiles or signatures for a given type of cancer in a particular patient population. Accordingly, knowledge of the activity level of a particular signal transduction system within a cancer cell prior to, during, and after treatment provides a physician with highly relevant information that can be used to select an appropriate course of treatment to adopt. Furthermore, the continued monitoring of signal transduction pathways that are active in cancer cells as treatment progresses can provide the physician with additional information on the efficacy of treatment, prompting the physician to either continue a particular course of treatment or to switch to another line of treatment, when, for example, cancer cells have become resistant to treatment through further aberrations that activate either the same or another signal transduction pathway.

Accordingly, the present invention provides methods for therapy selection by detecting, quantifying, and comparing the expression and activation states of a plurality of dysregulated signal transduction molecules in tumor tissue of a solid tumor in a specific, multiplex, high-throughput assay, such as the Collaborative Enzyme Enhanced Reactive Immunoassay (CEER). The present invention also provides methods for the selection of appropriate therapy (single drugs or combinations of drugs) to down-regulate or shut down a dysregulated signaling pathway. Thus, the invention can be used to facilitate the design of personalized therapies for cancer patients.

The ability to detect and quantify the activity of a plurality of signal transduction pathways in tumor cells characterized by dysregulated cMet signaling, and then to determine whether to administer a cMet inhibitor alone (e.g., monotherapy) or in combination (e.g., combination therapy) with a pathway-directed therapy based on upon differences in the expression and/or activation level of target proteins compared to control proteins and samples is an important advantage of the present invention. A current problem with selecting an effective therapeutic strategy for cancer patients is due to the high incidence of intrinsic and acquired resistance to anticancer drug treatments (e.g., tyrosine kinase inhibitors). For example, a subset of patients with non-small cell lung cancer are intrinsically resistant to EGFR TKIs. And even those initially responsive to the therapy tend to become resistant over time. The present invention overcomes or mitigates this and other problems by providing methods for the selection of appropriate therapy (single drugs or combinations of drugs) based on predictive expression/activation profiles of a plurality of dysregulated signal transduction molecules in tumor tissue of a solid tumor as determined by a specific, multiplex, high-throughput assay, such as a Collaborative Enzyme Enhanced Reactive Immunoassay (CEER). As such, the detection of the activation state of multiple signal transducers in rare cells in tumors facilitates cancer prognosis and diagnosis as well as the design of personalized, targeted therapies.

The methods of the present invention are beneficially tailored to address key issues in cancer management and provide a higher standard of care for malignant cancer patients because they (1) provide a method to detect and quantify the protein expression and/or activated protein level of components of multiple signaling pathways associated with cancer, (2) provide a method to compare the protein expression and/or activated protein levels to a control protein or control sample, (3) enable pathway profiling (e.g., expression and/or activation status of specific signal transduction molecules can be detected in tumor samples from patients), and (4) can be used as a predictive indicator of a patient's response to anticancer therapy. As such, the methods of the present invention enable the serial sampling of malignant tumor tissues, resulting in valuable information on changes occurring in tumor cells as a function of time and therapy and providing clinicians with a means to monitor rapidly evolving cancer pathway signatures.

In sum, the methods of the present invention advantageously provide accurate prediction, selection, and monitoring of cancer patients with aberrant cMet signaling most likely to benefit from targeted therapy by performing pathway profiling using, for example, multiplexed, antibody-based assays such as CEER, and comparing the pathway profiles to prognostic molecular profiles predictive of a patient's response to particular anticancer therapies.

### II. Definitions

As used herein, the following terms have the meanings ascribed to them unless specified otherwise.

The term “cancer” is intended to include any member of a class of diseases characterized by the uncontrolled growth of aberrant cells. The term includes all known cancers and neoplastic conditions, whether characterized as malignant, benign, soft tissue, or solid, and cancers of all stages and grades including pre- and post-metastatic cancers. Examples of different types of cancer include, but are not limited to, digestive and gastrointestinal cancers such as gastric cancer (e.g., stomach cancer), colorectal cancer, gastrointestinal stromal tumors (GIST), gastrointestinal carcinoid tumors, colon cancer, rectal cancer, anal cancer, bile duct cancer, small intestine cancer, and esophageal cancer; breast cancer; lung cancer (e.g., non-small cell lung cancer (NSCLC)); gallbladder cancer; liver cancer; pancreatic cancer; appendix cancer; prostate cancer, ovarian cancer; renal cancer (e.g., renal cell carcinoma); cancer of the central nervous system; skin cancer; lymphomas; gliomas; choriocarcinomas; head and neck cancers; osteogenic sarcomas; and blood cancers. As used herein, a “tumor” comprises one or more cancerous cells.

The term “non-small cell lung cancer” or “NSCLC” includes a disease in which malignant cancer cells form in the tissues of the lung. Examples of non-small cell lung cancers include, but are not limited to, squamous cell carcinoma, large cell carcinoma, and adenocarcinoma.

The term “analyte” includes any molecule of interest, typically a macromolecule such as a polypeptide, whose presence, amount (expression level), activation state, and/or identity is determined In certain instances, the analyte is a signal transduction molecule such as, e.g., a component of a HER3 (ErbB3) or cMet signaling pathway.

The term “signal transduction molecule” or “signal transducer” includes proteins and other molecules that carry out the process by which a cell converts an extracellular signal or stimulus into a response, typically involving ordered sequences of biochemical reactions inside the cell. Examples of signal transduction molecules include, but are not limited to, receptor tyrosine kinases such as EGFR (e.g., EGFR/HER1/ErbB1, HER2/Neu/ErbB2, HER3/ErbB3, HER4/ErbB4), VEGFR1/FLT1, VEGFR2/FLK1/KDR, VEGFR3/FLT4, FLT3/FLK2, PDGFR (e.g., PDGFRA, PDGFRB), c-KIT/SCFR, INSR (insulin receptor), IGF-IR, IGF-IIR, IRR (insulin receptor-related receptor), CSF-1R, FGFR 1-4, HGFR 1-2, CCK4, TRK A-C, c-MET, RON, EPHA 1-8, EPHB 1-6, AXL, MER, TYRO3, TIE 1-2, TEK, RYK, DDR 1-2, RET, c-ROS, V-cadherin, LTK (leukocyte tyrosine kinase), ALK (anaplastic lymphoma kinase), ROR 1-2, MUSK, AATYK 1-3, and RTK 106; truncated forms of receptor tyrosine kinases such as truncated HER2 receptors with missing amino-terminal extracellular domains (e.g., p95ErbB2 (p95m), p110, p95c, p95n, etc.), truncated cMET receptors with missing amino-terminal extracellular domains, and truncated HER3 receptors with missing amino-terminal extracellular domains; receptor tyrosine kinase dimers (e.g., p95HER2/HER3; p95HER2/HER2; truncated HER3 receptor with HER1, HER2, HER3, or HER4; HER2/HER2; HER3/HER3; HER2/HER3; HER1/HER2; HER1/HER3; HER2/HER4; HER3/HER4; etc.); non-receptor tyrosine kinases such as BCR-ABL, Src, Frk, Btk, Csk, Abl, Zap70, Fes/Fps, Fak, Jak, Ack, and LIMK; tyrosine kinase signaling cascade components such as AKT (e.g., AKT1, AKT2, AKT3), MEK (MAP2K1), ERK2 (MAPK1), ERK1 (MAPK3), PI3K (e.g., PIK3CA (p110), PIK3R1 (p85)), PDK1, PDK2, phosphatase and tensin homolog (PTEN), SGK3, 4E-BP1, P70S6K (e.g., p70 S6 kinase splice variant alpha I), protein tyrosine phosphatases (e.g., PTP1B, PTPN13, BDP1, etc.), RAF, PLA2, MEKK, JNKK, JNK, p38, Shc (p66), Ras (e.g., K-Ras, N-Ras, H-Ras), Rho, Rac1, Cdc42, PLC, PKC, p53, cyclin D1, STAT1, STAT3, phosphatidylinositol 4,5-bisphosphate (PIP2), phosphatidylinositol 3,4,5-trisphosphate (PIP3), mTOR, BAD, p21, p27, ROCK, IP3, TSP-1, NOS, GSK-3β, RSK 1-3, JNK, c-Jun, Rb, CREB, Ki67, and paxillin; nuclear hormone receptors such as estrogen receptor (ER), progesterone receptor (PR), androgen receptor, glucocorticoid receptor, mineralocorticoid receptor, vitamin A receptor, vitamin D receptor, retinoid receptor, thyroid hormone receptor, and orphan receptors; nuclear receptor coactivators and repressors such as amplified in breast cancer-1 (AIB1) and nuclear receptor corepressor 1 (NCOR), respectively; and combinations thereof.

The term “component of a HER3 signaling pathway” includes any one or more of an upstream ligand of HER3, binding partner of HER3, and/or downstream effector molecule that is modulated through HER3. Examples of HER3 signaling pathway components include, but are not limited to, heregulin, HER1/ErbB1, HER2/ErbB2, HER3/ErbB3, HER4/ErbB4, AKT (e.g., AKT1, AKT2, AKT3), MEK (MAP2K1), ERK2 (MAPK1), ERK1 (MAPK3), PI3K (e.g., PIK3CA (p110), PIK3R1 (p85)), PDK1, PDK2, PTEN, SGK3, 4E-BP1, P70S6K (e.g., splice variant alpha I), protein tyrosine phosphatases (e.g., PTP1B, PTPN13, BDP1, etc.), HER3 dimers (e.g., p95HER2/HER3, HER2/HER3, HER3/HER3, HER3/HER4, etc.), GSK-3β, PIP2, PIP3, p27, and combinations thereof.

The term “component of a c-Met signaling pathway” includes any one or more of an upstream ligand of c-Met, binding partner of c-Met, and/or downstream effector molecule that is modulated through c-Met. Examples of c-Met signaling pathway components include, but are not limited to, hepatocyte growth factor/scatter factor (HGF/SF), Plexin B1, CD44v6, AKT (e.g., AKT1, AKT2, AKT3), MEK (MAP2K1), ERK2 (MAPK1), ERK1 (MAPK3), STAT (e.g., STAT1, STAT3), PI3K (e.g., PIK3CA (p110), PIK3R1 (p85)), GRB2, Shc (p66), Ras (e.g., K-Ras, N-Ras, H-Ras), GAB1, SHP2, SRC, GRB2, CRKL, PLCγ, PKC (e.g., PKCα, PKCβ, PKCδ), paxillin, FAK, adducin, RB, RB 1, PYK2, and combinations thereof.

The term “aberrant c-Met signaling” refers to deregulation of one or more of the c-Met signaling pathway components due to causes such as, but not limited to, activation of the c-Met receptor via genomic alterations, changes in protein expression levels, changes in activated protein levels, and increased ligand stimulation. Examples of c-Met signaling pathway components include, but are not limited to, those described herein.

The term “truncated c-Met protein” includes a truncated form of the c-Met receptor that includes, but is not limited to, a protein containing the cytoplasmic and juxtamembrane domains of c-Met (see, e.g., Amicone et al., Gene, 162: 323-328 (1995) and Amicone et al., Oncogene, 21: 1335-1345); a protein containing the extracellular domain of c-Met; a protein comprising the alpha-chain and the 85-kDa C-terminal truncated beta-chain of c-Met (see, e.g., Prat et al., Mol. Cell. Biol. 11:5954-5962 (1991)); and a protein comprising the alpha-chain and the 75-kDa C-terminal truncated beta chain-of c-Met (see, e.g., Prat et al., Mol. Cell. Biol., 11:5954-5962 (1991)). In certain instances, the truncated receptor is typically a fragment of the full-length receptor and shares an intracellular domain (ICD) binding region with the full-length receptor. In certain embodiments, the full-length receptor comprises an extracellular domain (ECD) binding region, a transmembrane domain, and an intracellular domain (ICD) binding region. Without being bound to any particular theory, the truncated receptor may arise through the proteolytic processing of the ECD of the full-length receptor or by alternative initiation of translation from methionine residues that are located before, within, or after the transmembrane domain, e.g., to create a truncated c-Met receptor with a shortened ECD or a truncated c-Met receptor comprising a membrane-associated or cytosolic ICD fragment.

The term “truncated HER3 protein” includes a truncated form of the HER3 receptor that includes, but is not limited to, a protein containing the cytoplasmic and juxtamembrane domains of HER3; a truncated extracellular fragment of HER3 of 140 amino acids followed by 43 unique residues (see, e.g., Srinivasan et al., Cell Signal, 13:321-30 (2001)); and a 45-kDa glycosylated HER3 protein (see, e.g., Lin et al., Oncogene, 0.27:5195-5203 (2008)). In certain instances, the truncated receptor is typically a fragment of the full-length receptor and shares an intracellular domain (ICD) binding region with the full-length receptor. In certain embodiments, the full-length receptor comprises an extracellular domain (ECD) binding region, a transmembrane domain, and an intracellular domain (ICD) binding region. Without being bound to any particular theory, the truncated receptor may arise through the proteolytic processing of the ECD of the full-length receptor or by alternative initiation of translation from methionine residues that are located before, within, or after the transmembrane domain, e.g., to create a truncated HER3 receptor with a shortened ECD or a truncated HER3 receptor comprising a membrane-associated or cytosolic ICD fragment.

The term “activation state” refers to whether a particular signal transduction molecule such as a HER3 or c-Met signaling pathway component is activated. Similarly, the term “activation level” refers to what extent a particular signal transduction molecule such as a HER3 or c-Met signaling pathway component is activated. The activation state typically corresponds to the phosphorylation, ubiquitination, and/or complexation status of one or more signal transduction molecules. Non-limiting examples of activation states (listed in parentheses) include: HER1/EGFR (EGFRvIII, phosphorylated (p-) EGFR, EGFR:Shc, ubiquitinated (u-) EGFR, p-EGFRvIII); ErbB2 (p-ErbB2, p95HER2 (truncated ErbB2), p-p95HER2, ErbB2:Shc, ErbB2:PI3K, ErbB2:EGFR, ErbB2:ErbB3, ErbB2:ErbB4); ErbB3 (p-ErbB3, truncated ErbB3, ErbB3:PI3K, p-ErbB3:PI3K, ErbB3:Shc); ErbB4 (p-ErbB4, ErbB4:Shc); c-MET (p-c-MET, truncated c-MET, c-Met:HGF complex); AKT1 (p-AKT1); AKT2 (p-AKT2); AKT3 (p-AKT3); PTEN (p-PTEN); P70S6K (p-P70S6K); MEK (p-MEK); ERK1 (p-ERK1); ERK2 (p-ERK2); PDK1 (p-PDK1); PDK2 (p-PDK2); SGK3 (p-SGK3); 4E-BP1 (p-4E-BP1); PIK3R1 (p-PIK3R1); c-KIT (p-c-KIT); ER (p-ER); IGF-1R (p-IGF-1R, IGF-1R:IRS, IRS:PI3K, p-IRS, IGF-1R:PI3K); INSR (p-INSR); FLT3 (p-FLT3); HGFR1 (p-HGFR1); HGFR2 (p-HGFR2); RET (p-RET); PDGFRA (p-PDGFRA); PDGFRB (p-PDGFRB); VEGFR1 (p-VEGFR1, VEGFR1:PLCγ, VEGFR1:Src); VEGFR2 (p-VEGFR2, VEGFR2:PLCγ, VEGFR2:Src, VEGFR2:heparin sulphate, VEGFR2:VE-cadherin); VEGFR3 (p-VEGFR3); FGFR1 (p-FGFR1); FGFR2 (p-FGFR2); FGFR3 (p-FGFR3); FGFR4 (p-FGFR4); TIE1 (p-TIE1); TIE2 (p-TIE2); EPHA (p-EPHA); EPHB (p-EPHB); GSK-3β (p-GSK-3β); NFKB (p-NFKB), IKB (p-IKB, p-P65:IKB); BAD (p-BAD, BAD:14-3-3); mTOR (p-mTOR); Rsk-1 (p-Rsk-1); Jnk (p-Jnk); P38 (p-P38); STAT1 (p-STAT1); STAT3 (p-STAT3); FAK (p-FAK); RB (p-RB); Ki67; p53 (p-p53); CREB (p-CREB); c-Jun (p-c-Jun); c-Src (p-c-Src); paxillin (p-paxillin); GRB2 (p-GRB2), Shc (p-Shc), Ras (p-Ras), GAB1 (p-GAB1), SHP2 (p-SHP2), GRB2 (p-GRB2), CRKL (p-CRKL), PLCγ (p-PLCγ), PKC (e.g., p-PKCα, p-PKCβ, p-PKCδ), adducin (p-adducin), RB1 (p-RB1), and PYK2 (p-PYK2).

The term “KRAS mutation” includes any one or more mutations in the KRAS (which can also be referred to as KRAS2 or RASK2) gene. Examples of KRAS mutations include, but are not limited to, G12C, G12D, G13D, G12R, and G12V.

The term “EGFR mutation” includes any one or more mutations in the EGFR (which can also be referred to as ErbB1) gene. Examples of EGFR mutations include, but are not limited to, deletions in exon 19 such as L858R, G719S, G719S, G719C, L861Q and S768I, as well as insertions in exon 20, such as T790M.

The term “pathway-directed therapy” includes the use of therapeutic agents which can alter the expression level and/or activated level of proteins.

The term “cMet inhibitor” includes a therapeutic agent that interferes with the function of cMet pathway components. Examples of cMet inhibitors include, but are not limited to, neutralizing antibodies such as MAG102 (Amgen) and MetMab (Roche), and tyrosine kinase inhibitors (TKIs) such as ARQ197, XL184, PF-02341066, GSK1363089/XL880, MP470, MGCD265, SGX523, PF04217903 and JNJ38877605.

The term “EGFR inhibitor” includes a therapeutic agent that interferes with the function of EGFR pathway components. Non-limiting examples include Cetaximab, Panitumumab, Matuzumab, Nimotuzumab, ErbB1 vaccine, Erlotinib, Gefitinib, EKB 569, and CL-387-785.

The term “VEGFR inhibitor” includes a therapeutic agent that interferes with the function of the VEGF receptor pathways components, including but not limited to VEGF1/FLT1, VEGFR2/FLK1/KDR, VEGFR3/FLT4, AKT (e.g., AKT1, AKT2, AKT3), MEK (MAP2K1), ERK2 (MAPK1), ERK1 (MAPK3), STAT (e.g., STAT1, STAT3), PI3K (e.g., PIK3CA (p110), PIK3R1 (p85)), GRB2, Shc (p66), Ras (e.g., K-Ras, N-Ras, H-Ras), GAB1, SHP2, SRC, GRB2, CRKL, PLCγ, PKC (e.g., PKCα, PKCβ, PKCδ), paxillin, FAK, adducin, RB, RB1, PYK2, eNOS, HSP27, and combinations thereof. Non-limiting examples of VEGFR inhibitors include Bevacizumab (Avastin), HuMV833, VEGF-Trap, AZD 2171, AMG-706, Sunitinib (SU11248), Sorafenib (BAY43-9006), AE-941 (Neovastat) and Vatalanib (PTK787/ZK222584).

The term “serial changes” includes the ability of an assay to detect changes in the expression level and/or activation level of a protein in a sample taken from a subject at different points in time. For example, the expression level and/or activation level of cMet protein can be monitored in a patient during the course of therapy, including a time prior to starting therapy.

The term “negative response” includes a worsening of a disease condition in a patient receiving therapy, such that the patient experiences increased or additional signs or symptoms of the disease.

The term “positive response” includes an improvement in a patient with a disease condition, such that the therapy alleviates signs or symptoms of the disease.

The term “disease remission” includes a classification of a cancer wherein there is a disappearance in the signs and symptoms of the disease.

The term “disease progression” includes a classification of a cancer that continues to grow or spread, which can lead to additional signs or symptoms of cancer. For example, the recurrence of tumors in lung tissue in patients with NSCLC is described herein as disease progression.

The term “response rate” or “RR” includes the percentage of patients with positive responses, such as tumor shrinkage or disappearance, to a defined therapy for the treatment of a disease.

The term “complete response” or “complete remission” or “CR” includes the clinical endpoint described by the disappearance of all signs of cancer in response to treatment after a period of time. For example, if at the end of the time or treatment course, there is no residual disease that can be identified by measurements of symptom control and quality of life as performed by examination, X-ray and scan, or analysis of biomarkers of the disease, the patient is described herein to exhibit complete response to therapy. In certain instances, complete response is the disappearance of all tumor lesions (see, National Cancer Institute's Response Evaluation Criteria in Solid Tumors (RECIST), updated in January 2009).

The term “partial response” or “PR” includes a clinical endpoint described as the disappearance of some, but not all signs of cancer in response to treatment after a period of time. For example, if at the end of the time or treatment course, there is some detectable residual disease that can be identified by measurements of symptom control and quality of life as performed by examination, X-ray and scan, or analysis of biomarkers of the disease, the patient is described herein to exhibit partial response to therapy. In certain instances, partial response is a 30% decrease in the sum of the longest diameter of the tumor lesions (see, National Cancer Institute's Response Evaluation Criteria in Solid Tumors (RECIST), updated in January 2009).

The term “stable disease” or “SD” includes a clinical endpoint in cancer characterized by the appearance of no new tumors and no substantial change in the size of existing, known tumors. According to RECIST, stable disease is defined as small changes that do not meet the criteria of complete response, partial response, and progressive disease (which is defined as a 20% increase in the sum of the longest diameter of the tumor lesions).

The term “time to progression” or “TTP” includes the measure of time after a disease is diagnosed or treated until the disease starts to worsen (e.g., appearance of new tumors, increase in tumor size; change in the quality of life, or change in symptom control).

The term “progression free survival” or “PFS” includes the length of time during and after a treatment of a disease in which a patient is living with the disease without additional symptoms of the disease.

The term “overall survival” or “OS” includes the clinical endpoint describing patients who are alive for a defined period of time after being diagnosed with or treated for a disease, such as cancer.

As used herein, the term “dilution series” is intended to include a series of descending concentrations of a particular sample (e.g., cell lysate) or reagent (e.g., antibody). A dilution series is typically produced by a process of mixing a measured amount of a starting concentration of a sample or reagent with a diluent (e.g., dilution buffer) to create a lower concentration of the sample or reagent, and repeating the process enough times to obtain the desired number of serial dilutions. The sample or reagent can be serially diluted at least 2, 3, 4, 5, 6, 7, 8, 9, 10, 15, 20, 25, 30, 35, 40, 45, 50, 100, 500, or 1000-fold to produce a dilution series comprising at least 2, 3, 4, 5, 6, 7, 8, 9, 10, 11, 12, 13, 14, 15, 16, 17, 18, 19, 20, 25, 30, 35, 40, 45, or 50 descending concentrations of the sample or reagent. For example, a dilution series comprising a 2-fold serial dilution of a capture antibody reagent at a 1 mg/ml starting concentration can be produced by mixing an amount of the starting concentration of capture antibody with an equal amount of a dilution buffer to create a 0.5 mg/ml concentration of the capture antibody, and repeating the process to obtain capture antibody concentrations of 0.25 mg/ml, 0.125 mg/ml, 0.0625 mg/ml, 0.0325 mg/ml, etc.

The term “superior dynamic range” as used herein refers to the ability of an assay to detect a specific analyte in as few as one cell or in as many as thousands of cells. For example, the immunoassays described herein possess superior dynamic range because they advantageously detect a particular signal transduction molecule of interest in about 1-10,000 cells (e.g., about 1, 5, 10, 25, 50, 75, 100, 250, 500, 750, 1000, 2500, 5000, 7500, or 10,000 cells) using a dilution series of capture antibody concentrations.

As used herein, the term “circulating cells” comprises extratumoral cells that have either metastasized or micrometastasized from a solid tumor. Examples of circulating cells include, but are not limited to, circulating tumor cells, cancer stem cells, and/or cells that are migrating to the tumor (e.g., circulating endothelial progenitor cells, circulating endothelial cells, circulating pro-angiogenic myeloid cells, circulating dendritic cells, etc.). Patient samples containing circulating cells can be obtained from any accessible biological fluid (e.g., whole blood, serum, plasma, sputum, bronchial lavage fluid, urine, nipple aspirate, lymph, saliva, fine needle aspirate, etc.). In certain instances, the whole blood sample is separated into a plasma or serum fraction and a cellular fraction (i.e., cell pellet). The cellular fraction typically contains red blood cells, white blood cells, and/or circulating cells of a solid tumor such as circulating tumor cells (CTCs), circulating endothelial cells (CECs), circulating endothelial progenitor cells (CEPCs), cancer stem cells (CSCs), disseminated tumor cells of the lymph node, and combinations thereof. The plasma or serum fraction usually contains, inter alia, nucleic acids (e.g., DNA, RNA) and proteins that are released by circulating cells of a solid tumor.

Circulating cells are typically isolated from a patient sample using one or more separation methods including, for example, immunomagnetic separation (see, e.g., Racila et al., Proc. Natl. Acad. Sci. USA, 95:4589-4594 (1998); Bilkenroth et al., Int. J. Cancer, 92:577-582 (2001)), the CellTracks® System by Immunicon (Huntingdon Valley, Pa.), microfluidic separation (see, e.g., Mohamed et al., IEEE Trans. Nanobiosci., 3:251-256 (2004); Lin et al., Abstract No. 5147, 97th AACR Annual Meeting, Washington, D.C. (2006)), FACS (see, e.g., Mancuso et al., Blood, 97:3658-3661 (2001)), density gradient centrifugation (see, e.g., Baker et al., Clin. Cancer Res., 13:4865-4871 (2003)), and depletion methods (see, e.g., Meye et al., Int. J. Oncol., 21:521-530 (2002)).

The term “sample” as used herein includes any biological specimen obtained from a patient. Samples include, without limitation, whole blood, plasma, serum, red blood cells, white blood cells (e.g., peripheral blood mononuclear cells), ductal lavage fluid, nipple aspirate, lymph (e.g., disseminated tumor cells of the lymph node), bone marrow aspirate, saliva, urine, stool (i.e., feces), sputum, bronchial lavage fluid, tears, fine needle aspirate (e.g., harvested by random periareolar fine needle aspiration), any other bodily fluid, a tissue sample (e.g., tumor tissue) such as a biopsy of a tumor (e.g., needle biopsy) or a lymph node (e.g., sentinel lymph node biopsy), a tissue sample (e.g., tumor tissue) such as a surgical resection of a tumor, and cellular extracts thereof. In some embodiments, the sample is whole blood or a fractional component thereof such as plasma, serum, or a cell pellet. In preferred embodiments, the sample is obtained by isolating circulating cells of a solid tumor from whole blood or a cellular fraction thereof using any technique known in the art. In other embodiments, the sample is a formalin fixed paraffin embedded (FFPE) tumor tissue sample, e.g., from a solid tumor of the stomach or other portion of the gastrointestinal tract.

A “biopsy” refers to the process of removing a tissue sample for diagnostic or prognostic evaluation, and to the tissue specimen itself. Any biopsy technique known in the art can be applied to the methods and compositions of the present invention. The biopsy technique applied will generally depend on the tissue type to be evaluated and the size and type of the tumor (i.e., solid or suspended (i.e., blood or ascites)), among other factors. Representative biopsy techniques include excisional biopsy, incisional biopsy, needle biopsy (e.g., core needle biopsy, fine-needle aspiration biopsy, etc.), surgical biopsy, and bone marrow biopsy. Biopsy techniques are discussed, for example, in Harrison's Principles of Internal Medicine, Kasper, et al., eds., 16th ed., 2005, Chapter 70, and throughout Part V. One skilled in the art will appreciate that biopsy techniques can be performed to identify cancerous and/or precancerous cells in a given tissue sample.

The term “subject” or “patient” or “individual” typically includes humans, but can also include other animals such as, e.g., other primates, rodents, canines, felines, equines, ovines, porcines, and the like.

The term “Caucasian” includes a human classification of persons of non-Hispanic European descent. For example, a person having origins in any of the original peoples of Europe, the Middle East, or North Africa.

The term “Asian” includes a human classification of persons who descend from an ethnic groups in Asia. For example, a person having origins in any of the original peoples of the Far East, Southeast Asia, or the Indian subcontinent, including, for example, Cambodia, China, India, Japan, Korea, Malaysia, Pakistan, the Philippine Islands, Thailand, and Vietnam.

An “array” or “microarray” comprises a distinct set and/or dilution series of capture antibodies immobilized or restrained on a solid support such as, for example, glass (e.g., a glass slide), plastic, chips, pins, filters, beads (e.g., magnetic beads, polystyrene beads, etc.), paper, membrane (e.g., nylon, nitrocellulose, polyvinylidene fluoride (PVDF), etc.), fiber bundles, or any other suitable substrate. The capture antibodies are generally immobilized or restrained on the solid support via covalent or noncovalent interactions (e.g., ionic bonds, hydrophobic interactions, hydrogen bonds, Van der Waals forces, dipole-dipole bonds). In certain instances, the capture antibodies comprise capture tags which interact with capture agents bound to the solid support. The arrays used in the assays described herein typically comprise a plurality of different capture antibodies and/or capture antibody concentrations that are coupled to the surface of a solid support in different known/addressable locations.

The term “capture antibody” is intended to include an immobilized antibody which is specific for (i.e., binds, is bound by, or forms a complex with) one or more analytes of interest in a sample such as a cellular extract. In particular embodiments, the capture antibody is restrained on a solid support in an array. Suitable capture antibodies for immobilizing any of a variety of signal transduction molecules on a solid support are available from Upstate (Temecula, Calif.), Biosource (Camarillo, Calif.), Cell Signaling Technologies (Danvers, Mass.), R&D Systems (Minneapolis, Minn.), Lab Vision (Fremont, Calif.), Santa Cruz Biotechnology (Santa Cruz, Calif.), Sigma (St. Louis, Mo.), and BD Biosciences (San Jose, Calif.).

The term “detection antibody” as used herein includes an antibody comprising a detectable label which is specific for (i.e., binds, is bound by, or forms a complex with) one or more analytes of interest in a sample. The term also encompasses an antibody which is specific for one or more analytes of interest, wherein the antibody can be bound by another species that comprises a detectable label. Examples of detectable labels include, but are not limited to, biotin/streptavidin labels, nucleic acid (e.g., oligonucleotide) labels, chemically reactive labels, fluorescent labels, enzyme labels, radioactive labels, and combinations thereof. Suitable detection antibodies for detecting the activation state and/or total amount of any of a variety of signal transduction molecules are available from Upstate (Temecula, Calif.), Biosource (Camarillo, Calif.), Cell Signaling Technologies (Danvers, Mass.), R&D Systems (Minneapolis, Minn.), Lab Vision (Fremont, Calif.), Santa Cruz Biotechnology (Santa Cruz, Calif.), Sigma (St. Louis, Mo.), and BD Biosciences (San Jose, Calif.). As a non-limiting example, phospho-specific antibodies against various phosphorylated forms of signal transduction molecules such as EGFR, c-KIT, c-Src, FLK-1, PDGFRA, PDGFRB, AKT, MAPK, PTEN, Raf, and MEK are available from Santa Cruz Biotechnology.

The term “activation state-dependent antibody” includes a detection antibody which is specific for (i.e., binds, is bound by, or forms a complex with) a particular activation state of one or more analytes of interest in a sample. In preferred embodiments, the activation state-dependent antibody detects the phosphorylation, ubiquitination, and/or complexation state of one or more analytes such as one or more signal transduction molecules. In some embodiments, the phosphorylation of members of the EGFR family of receptor tyrosine kinases and/or the formation of heterodimeric complexes between EGFR family members is detected using activation state-dependent antibodies. In particular embodiments, activation state-dependent antibodies are useful for detecting one or more sites of phosphorylation in one or more of the following signal transduction molecules (phosphorylation sites correspond to the position of the amino acid in the human protein sequence): EGFR/HER1/ErbB1 (e.g., tyrosine (Y) 1068); ErbB2/HER2 (e.g., Y1248); ErbB3/HER3 (e.g., Y1289); ErbB4/HER4 (e.g., Y1284); c-Met (e.g., Y1003, Y1230, Y1234, Y1235, and/or Y1349); SGK3 (e.g., threonine (T) 256 and/or serine (S) 422); 4E-BP1 (e.g., T70); ERK1 (e.g., T185, Y187, T202, and/or Y204); ERK2 (e.g., T185, Y187, T202, and/or Y204); MEK (e.g., S217 and/or S221); PIK3R1 (e.g., Y688); PDK1 (e.g., S241); P70S6K (e.g., T229, T389, and/or S421); PTEN (e.g., S380); AKT1 (e.g., S473 and/or T308); AKT2 (e.g., S474 and/or T309); AKT3 (e.g., S472 and/or T305); GSK-33 (e.g., S9); NFKB (e.g., S536); IKB (e.g., S32); BAD (e.g., S112 and/or S136); mTOR (e.g., S2448); Rsk-1 (e.g., T357 and/or S363); Jnk (e.g., T183 and/or Y185); P38 (e.g., T180 and/or Y182); STAT3 (e.g., Y705 and/or S727); FAK (e.g., Y397, Y576, S722, Y861, and/or S910); RB (e.g., S249, T252, S612, and/or S780); RB1 (e.g., S780); adducin (e.g., S662 and/or S724); PYK2 (e.g., Y402 and/or Y881); PKCα (e.g., S657); PKCα/β (e.g., T368 and/or T641); PKCδ (e.g., T505); p53 (e.g., S392 and/or S20); CREB (e.g., S133); c-Jun (e.g., S63); c-Src (e.g., Y416); and paxillin (e.g., Y31 and/or Y118).

The term “activation state-independent antibody” includes a detection antibody which is specific for (i.e., binds, is bound by, or forms a complex with) one or more analytes of interest in a sample irrespective of their activation state. For example, the activation state-independent antibody can detect both phosphorylated and unphosphorylated forms of one or more analytes such as one or more signal transduction molecules.

Non-limiting examples of activation state-independent c-Met antibodies include those available under the following catalog numbers: AF276, MAB3581, MAB3582, MAB3583, and MAB5694 (R&D Systems); ab51067, ab39075, ab47431, ab14571, ab10728, ab59884, ab47431, ab49210, ab71758, ab74217, ab47465, ab27492 (Abeam); SC161HRP (Santa Cruz); PA1-14257, PA1-37483, and PA1-37484 (Thermo Scientific); 05-1049, MAB3729 (Millipore); sc-162, sc-34405, sc-161, sc-10, sc-8307, sc-46394, sc-46395, sc-81478 (Santa Cruz Biotechnology); 8198, 3127, 3148, and 4560 (Cell Signaling Technology); and 700261, 370100, 718000 (Life Technologies).

Non-limiting examples of activation state-dependent cMET antibodies include those available under the following catalog numbers: AF2480, AF3950, AF4059 (R&D Systems); PA1-14254, PA1-14256 (Thermo Scientific); sc-16315, sc-34086, sc34085, sc-34087, sc-101736, sc-101737 (Santa Cruz Biotechnology); ab61024, ab5662, ab73992, and ab5656 (Abeam); 3135, 3077, 3129, 3126, 3133, 3121 (Cell Signaling Technology); and 44892G, 44896G, 700139, 44887G, 44888G, 44882G (Life Technologies).

Non-limiting examples of capture antibodies that recognize cMET include those available under the following catalog numbers: AF276, MAB3581, MAB3582, MAB3583, and MAB5694 (R&D Systems); ab51067, ab39075, ab47431, ab14571, ab10728, ab59884, ab47431, ab49210, ab71758, ab74217, ab47465, ab27492 (Abeam); SC161HRP (Santa Cruz); PA1-14257, PA1-37483, and PA1-37484 (Thermo Scientific); 05-1049, MAB3729 (Millipore); sc-162, sc-34405, sc-161, sc-10, sc-8307, sc-46394, sc-46395, sc-81478 (Santa Cruz Biotechnology); 8198, 3127, 3148, and 4560 (Cell Signaling Technology); and 700261, 370100, 718000 (Life Technologies).

Non-limiting examples of activation state-independent HER3 antibodies include those available under the following catalog numbers: PA1-86644 and MS-201-PABX (Thermo Scientific); MAB348, MAB3481, MAB3482, MAB3483 (R&D Systems); sc-415, sc-53279, sc-23865, sc-7390, sc-71067, sc-71066, sc-56385, sc-285, sc-71068, sc-81454, sc81455 (Santa Cruz Technologies); and 44897M (Life Technologies).

Non-limiting examples of activation state-dependent HER3 antibodies include those available under the following catalog numbers: AF5817 (R&D Systems); sc-135654 (Santa Cruz Biotechnology); 8017, 4561, 4784, 4791, and 4754 (Cell Signaling Technology); and 44901M (Life Technologies).

Non-limiting examples of capture antibodies that recognize HER3 include those available under the following catalog numbers: PA1-86644 and MS-201-PABX (Thermo Scientific); MAB348, MAB3481, MAB3482, MAB3483 (R&D Systems); sc-415, sc-53279, sc-23865, sc-7390, sc-71067, sc-71066, sc-56385, sc-285, sc-71068, sc-81454, sc81455 (Santa Cruz Technologies); and 44897M (Life Technologies).

Examples of activation state-dependent antibodies that recognize phosphotyrosine residues include, but are not limited to, 4G10 Anti-Phosphotyrosine antibody from Millipore; the Anti-Phosphotyrosine antibody [PY20] (ab10321) from Abcam plc; the DELFIA Eu-N1 Anti-Phosphotyrosine P-Tyr-100, PT66, and PY20 antibodies from PerkinElmer Inc.; and the Anti-Phosphotyrosine PY20, PT-66, and PT-154 monoclonal antibodies from Sigma-Aldrich Co. Examples of activation state-dependent antibodies that recognize phosphoserine residues include, but are not limited to, PSR-45 monoclonal antibody from Sigma-Aldrich Co.; Anti-Phosphoserine antibody [PSR-45] (ab6639) from Abcam plc; Anti-Phosphoserine clone 4A4 from Millipore; Phosphoserine Antibody (NB600-558) from Novus Biologicals; the DELFIA Eu-N1 labeled anti-phosphoserine antibody from PerkinElmer Inc.; and the PhosphoSerine Antibody Q5 from Qiagen. Examples of activation state-dependent antibodies that recognize phosphothreonine residues include, but are not limited to, PTR-8 monoclonal antibody P3555 from Sigma-Aldrich Co.; Phospho-Threonine Antibody (P-Thr-Polyclonal) #9381 from Cell Signaling Technology; Anti-Phosphothreonine antibody (ab9337) from Abcam plc; and the PhosphoThreonine Antibody Q7 from Qiagen.

The term “nucleic acid” or “polynucleotide” includes deoxyribonucleotides or ribonucleotides and polymers thereof in either single- or double-stranded form such as, for example, DNA and RNA. Nucleic acids include nucleic acids containing known nucleotide analogs or modified backbone residues or linkages, which are synthetic, naturally occurring, and non-naturally occurring, and which have similar binding properties as the reference nucleic acid. Examples of such analogs include, without limitation, phosphorothioates, phosphoramidates, methyl phosphonates, chiral-methyl phosphonates, 2′-O-methyl ribonucleotides, and peptide-nucleic acids (PNAs). Unless specifically limited, the term encompasses nucleic acids containing known analogues of natural nucleotides that have similar binding properties as the reference nucleic acid. Unless otherwise indicated, a particular nucleic acid sequence also implicitly encompasses conservatively modified variants thereof and complementary sequences as well as the sequence explicitly indicated.

The term “oligonucleotide” includes a single-stranded oligomer or polymer of RNA, DNA, RNA/DNA hybrid, and/or a mimetic thereof. In certain instances, oligonucleotides are composed of naturally-occurring (i.e., unmodified) nucleobases, sugars, and internucleoside (backbone) linkages. In certain other instances, oligonucleotides comprise modified nucleobases, sugars, and/or internucleoside linkages.

As used herein, the term “mismatch motif” or “mismatch region” refers to a portion of an oligonucleotide that does not have 100% complementarity to its complementary sequence. An oligonucleotide may have at least one, two, three, four, five, six, or more mismatch regions. The mismatch regions may be contiguous or may be separated by 1, 2, 3, 4, 5, 6, 7, 8, 9, 10, 11, 12, or more nucleotides. The mismatch motifs or regions may comprise a single nucleotide or may comprise two, three, four, five, or more nucleotides.

The phrase “stringent hybridization conditions” refers to conditions under which an oligonucleotide will hybridize to its complementary sequence, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may also be achieved with the addition of destabilizing agents such as formamide. For selective or specific hybridization, a positive signal is at least two times background, preferably 10 times background hybridization.

The terms “substantially identical” or “substantial identity,” in the context of two or more nucleic acids, refer to two or more sequences or subsequences that are the same or have a specified percentage of nucleotides that are the same (i.e., at least about 60%, preferably at least about 65%, 70%, 75%, 80%, 85%, 90%, or 95% identity over a specified region) when compared and aligned for maximum correspondence over a comparison window or designated region as measured using a sequence comparison algorithm or by manual alignment and visual inspection. This definition, when the context indicates, also refers analogously to the complement of a sequence. Preferably, the substantial identity exists over a region that is at least about 5, 10, 15, 20, 25, 30, 35, 40, 45, 50, 75, or 100 nucleotides in length.

The term “incubating” is used synonymously with “contacting” and “exposing” and does not imply any specific time or temperature requirements unless otherwise indicated.

“Receptor tyrosine kinases” or “RTKs” include a family of fifty-six (56) proteins characterized by a transmembrane domain and a tyrosine kinase motif. RTKs function in cell signaling and transmit signals regulating growth, differentiation, adhesion, migration, and apoptosis. The mutational activation and/or overexpression of receptor tyrosine kinases transforms cells and often plays a crucial role in the development of cancers. RTKs have become targets of various molecularly targeted agents such as trastuzumab, cetuximab, gefitinib, erlotinib, sunitinib, imatinib, nilotinib, and the like. One well-characterized signal transduction pathway is the MAP kinase pathway, which is responsible for transducing the signal from epidermal growth factor (EGF) to the promotion of cell proliferation in cells.

### III. Description of the Embodiments

The present invention provides methods for detecting the status (e.g., expression and/or activation levels) of components of signal transduction pathways in tumor cells derived from tumor tissue or circulating cells of a solid tumor with an assay such as a specific, multiplex, high-throughput proximity assay as described herein. The present invention also provides methods for selecting appropriate therapies to downregulate one or more deregulated signal transduction pathways. Thus, certain embodiments of the invention may be used to facilitate the design of personalized therapies based on the particular molecular signature provided by the collection of total and activated signal transduction proteins in a given patient's tumor (e.g., a malignancy involving aberrant cMet signaling).

In particular aspects, the present invention provides molecular markers (biomarkers) that enable the determination or prediction of whether a particular cancer can respond or is likely to respond favorably to an anticancer drug such as, for example, a EGFR modulating compound (e.g., a EGFR inhibitor), a VEGFR-modulating compound (e.g., a VEGFR inhibitor), and/or a cMet-modulating compound (e.g., a cMet inhibitor). In specific embodiments, measuring the level of expression and/or activation of one or more components of the HER3 and/or cMet signaling pathways (e.g., cMet, truncated cMet receptor, HER1, HER2, p95HER2, HER3, truncated HER3 receptor, HER4, IGF1R, cKit, PI3K, Shc, Akt, p70S6K, VEGFR1-3, and/or PDGFR) is particularly useful for selecting a suitable anticancer drug and/or identifying or predicting a response thereto in cells such as malignant cancer cells.

In one aspect, the present invention provides a method for therapy selection for a subject with a malignancy involving aberrant cMet signaling, the method comprising:
(a) detecting and/or quantifying the expression level and/or activation level of cMet protein in a sample taken from the subject;
(b) detecting and/or quantifying the expression level and/or activation level of HER3 protein in the sample;
(c) comparing the expression level and/or activation level of cMet protein and/or HER3 protein in the sample to (i) the expression level and/or activation level of a control protein and/or (ii) the expression level and/or activation level of cMet protein and/or HER3 protein in a control sample; and
(d) determining whether to administer a cMet inhibitor alone or a cMet inhibitor in combination with a pathway-directed therapy based upon a difference between the expression level and/or activation level of cMet protein and/or HER3 protein in the sample compared to the control protein and/or control sample.

In some embodiments, the expression (e.g., total) level and/or activation (e.g., phosphorylation) level of the one or more analytes is expressed as a relative fluorescence unit (RFU) value that corresponds to the signal intensity for a particular analyte of interest that is determined using, e.g., a proximity assay such as the Collaborative Enzyme Enhanced Reactive ImmunoAssay (CEER) described herein. In other embodiments, the expression level and/or activation level of the one or more analytes is quantitated by calibrating or normalizing the RFU value that is determined using, e.g., a proximity assay such as CEER, against a standard curve generated for the particular analyte of interest. In certain instances, the RFU value can be calculated based upon a standard curve.

In further embodiments, the expression level and/or activation level of the one or more analytes is expressed as “low,” “medium,” or “high” that corresponds to increasing signal intensity for a particular analyte of interest that is determined using, e.g., a proximity assay such as CEER. In some instances, an undetectable or minimally detectable level of expression or activation of a particular analyte of interest that is determined using, e.g., a proximity assay such as CEER, may be expressed as “undetectable.” In other instances, a low level of expression or activation of a particular analyte of interest that is determined using, e.g., a proximity assay such as CEER, may be expressed as “low.” In yet other instances, a moderate level of expression or activation of a particular analyte of interest that is determined using, e.g., a proximity assay such as CEER, may be expressed as “medium.” In still yet other instances, a moderate to high level of expression or activation of a particular analyte of interest that is determined using, e.g., a proximity assay such as CEER, may be expressed as “medium to high.” In further instances, a very high level of expression or activation of a particular analyte of interest that is determined using, e.g., a proximity assay such as CEER, may be expressed as “high.”

In certain embodiments, the expression (e.g., total) level or activation (e.g., phosphorylation) level of a particular analyte of interest, when expressed as “low,” “medium,” or “high,” may correspond to a level of expression or activation that is at least about 0; 5,000; 10,000; 15,000; 20;000; 25,000; 30,000; 35,000; 40,000; 45,000; 50,000; 60,000; 70;000; 80,000; 90,000; 100,000 RFU; or more, e.g., when compared to a negative control such as an IgG control, when compared to a standard curve generated for the analyte of interest, when compared to a positive control such as a pan-CK control, when compared to an expression or activation level determined in the presence of an anticancer drug, and/or when compared to an expression or activation level determined in the absence of an anticancer drug. In some instances, the correlation is analyte-specific. As a non-limiting example, a “low” level of expression or activation determined using, e.g., a proximity assay such as CEER, may correspond 10,000 RFUs in expression or activation for one analyte and 50,000 RFUs for another analyte when compared to a reference expression or activation level.

In certain embodiments, the expression or activation level of a particular analyte of interest may correspond to a level of expression or activation referred to as “low,” “medium,” or “high” that is relative to a reference expression level or activation level, e.g., when compared to a negative control such as an IgG control, when compared to a standard curve generated for the analyte of interest, when compared to a positive control such as a pan-CK control, when compared to an expression or activation level determined in the presence of an anticancer drug, and/or when compared to an expression or activation level determined in the absence of an anticancer drug. In some instances, the correlation is analyte-specific. As a non-limiting example, a “low” level of expression or activation determined using, e.g., a proximity assay such as CEER, may correspond to a 2-fold increase in expression or activation for one analyte and a 5-fold increase for another analyte when compared to a reference expression or activation level.

In certain embodiments, the expression or activation level of a particular analyte of interest may correspond to a level of expression or activation that is compared to a negative control such as an IgG control (i.e., control protein), compared to a standard curve generated for the analyte of interest, compared to a positive control such as a pan-CK control (i.e., control protein), compared to an expression or activation level determined in the presence of an anticancer drug (i.e., control sample), and/or compared to an expression or activation level determined in the absence of an anticancer drug (i.e., control sample). In particular embodiments, a control sample can be derived from a cell line or a tissue sample free from a malignancy involving aberrant cMet signaling.

In preferred embodiments, pathway-directed therapy is a therapeutic agent which can alter the expression and/or activation of signaling pathway components, such as, but not limited to, pan-HER inhibitors, EGFR inhibitors, cMet inhibitors, and VEGFR inhibitors. Non-limiting examples of pan-HER inhibitors include PF-00299804, neratinib (HKI-272), AC480 (BMS-599626), BMS-690154, PF-02341066, HM781-36B, CI-1033, BIBW-2992, and combinations thereof. Examples of HER2 inhibitors include, but are not limited to, monoclonal antibodies such as trastuzumab (Herceptin®) and pertuzumab (2C4); small molecule tyrosine kinase inhibitors such as gefitinib)(Iressa®, erlotinib)(Tarceva®, pelitinib, CP-654577, CP-724714, canertinib (CI 1033), HKI-272, lapatinib (GW-572016; Tykerb®), PKI-166, AEE788, BMS-599626, HKI-357, BIBW 2992, ARRY-380, ARRY-334543, CUDC-101, JNJ-26483327, and JNJ-26483327; and combinations thereof. Non-limiting examples of EGFR inhibitors include Cetaximab, Panitumumab, Matuzumab, Nimotuzumab, ErbB1 vaccine, Erlotinib, Gefitinib, ARRY-334543, AEE788, BIBW 2992, EKB 569, CL-387-785, CUDC-101, AV-412, and combinations thereof. Non-limiting examples of VEGFR inhibitors include Bevacizumab (Avastin), HuMV833, VEGF-Trap, AV-952, AZD 2171, AMG-706, Sunitinib (SU11248), Sorafenib (BAY43-9006), AE-941 (Neovastat), Vatalanib (PTK787/ZK222584), GSK-1363089, PTK-787, XL-800, ZD6474, AG13925, AG013736, CEP-7055, CP-547,632, GW786024, GW654652, GSK-VEG1003, GW786034B, and combinations thereof.

In some embodiments, a cMet inhibitor is selected from a group consisting of a multi-kinase inhibitor, a tyrosine kinase inhibitor, and a monoclonal antibody. In particular aspects, a multi-kinase inhibitor (e.g., pan-HER inhibitor) is an agent that blocks a plurality of kinases, such as but not limited to cMet, RON, EGFR, HER2, HER3, VEGFR1, VEGR2, VEGFR3, PI3K, SHC, p95HER2, IGF-1R, and/or c-Kit. In other aspects, a tyrosine kinase inhibitor is an agent that blocks specifically tyrosine kinases selected from a group consisting of, but not limited to, cMet, RON, EGFR, HER2, HER3, VEGFR1, VEGR2 and/or VEGFR3. Non-limiting examples of small molecule tyrosine kinase inhibitors which can be used in the present invention include a gefitinib (Iressa®), erlotinib (Tarceva®), pelitinib, CP-654577, CP-724714, canertinib (CI 1033), HKI-272, lapatinib (GW-572016; Tykerb®), PKI-166, AEE788, BMS-599626, HKI-357, BIBW 2992, ARRY-334543, JNJ-26483327, and JNJ-26483327; and combinations thereof. Non-limiting examples of monoclonal antibodies for use in the present invention include trastuzumab (Herceptin®), pertuzumab (2C4), alemtuzumab (Campath®), bevacizumab (Avastin®), cetuximab (Erbitux®), gemtuzumab (Mylotarg®), panitumumab (Vectibix™), rituximab (Rituxan®), and tositumomab (BEXXAR®), and combinations thereof. Non-limiting examples of pan-HER inhibitors, HER2 inhibitors, EGFR inhibitors, and VEGFR inhibitors are described above.

Non-limiting examples of compounds that modulate cMet activity are described herein and include monoclonal antibodies, small molecule inhibitors, and combinations thereof. In preferred embodiments, the cMet-modulating compound inhibits cMet activity and/or blocks cMet signaling, e.g., is a cMet inhibitor. Examples of cMet inhibitors include, but are not limited to, monoclonal antibodies such as AV299, L2G7, AMG102, DN30, OA-5D5, and MetMAb; and small molecule inhibitors of cMet such as ARQ197, AMG458, BMS-777607, XL 184, XL880, INCB28060, E7050, GSK1363089/XL880, K252a, LY2801653, MP470, MGCD265, MK-2461, NK2, NK4, SGX523, SU11274, SU5416, PF-04217903, PF-02341066, PHA-665752, JNJ-38877605; and combinations thereof.

In specific embodiments, the patient with a malignancy involving aberrant cMet signaling will possess tumor tissue with one or a plurality of KRAS mutations. A KRAS mutation can be a member selected from a group of mutations consisting of G12C, G12D, G13D, G12R, G12V, and combinations thereof.

In certain instances, the malignancy involving aberrant cMet signaling comprises a carcinoma of the breast, liver, lung, gastric, ovary, kidney, thyroid, or combinations thereof. In certain other instances, the malignant cancer involving aberrant cMet signaling is non-small cell lung cancer (NSCLC). In certain embodiments, NSCLC is any type of epithelial lung cancer other than small cell lung carcinoma. As is known to those skilled in the art, the most common types of NSCLC are squamous cell carcinoma, large cell carcinoma, and adenocarcinoma.

In some embodiments, step (d) comprises determining that the cMet inhibitor should be administered alone when the expression level and/or activation level of cMet protein in the sample is determined to range from medium to high compared to the control protein and/or control sample. In particular embodiments, the subject with a medium to high level of expression and/or activation of cMet protein is a Caucasian subject. In certain embodiments, the activation level of cMet protein corresponds to the level of phosphorylated cMet protein.

In other embodiments, step (d) comprises determining that the cMet inhibitor should be administered alone when the expression level and/or activation level of HER3 protein in the sample is determined to range from medium to high compared to the control protein and/or control sample. In particular embodiments, the subject with a medium to high level of HER3 expression and/or activation is a Caucasian subject. In certain embodiments, the activation level of HER3 protein corresponds to the level of phosphorylated HER3 protein.

In further embodiments, step (d) comprises determining that the cMet inhibitor should be administered alone when the expression level and/or activation level of cMet protein and HER3 protein in the sample is each independently determined to range from medium to high compared to the control protein and/or control sample. In certain other embodiments, the subject with a medium to high level of expression and/or activation of cMet protein and HER3 protein is a Caucasian subject. As such, in these embodiments, determining whether the cMet inhibitor should be administered alone in step (d) comprises determining whether expression levels and/or activation levels of cMet protein and HER3 both lie within the range of medium to high compared to the control protein and/or control sample. As described above, in certain preferred embodiments, the expression levels and/or activation levels of the analytes may be determined using a proximity assay such as CEER and expressed in RFU values which can be described as being “low,” “medium,” or “high.”

In yet other embodiments of the invention, determining whether the cMet inhibitor should be administered alone in step (d) comprises determining whether expression levels and/or activation levels of cMet and HER3 proteins in the sample both are within the range of medium to high compared to the control protein and/or control sample, and whether the expression levels of both HER1 and HER2 protein in the sample is low compared to the control protein and/or control sample. In particular instances, an expression/activation profile of a subject's tumor sample described as having medium to high levels of cMet and HER3 and low levels of HER1 and HER2 can indicate to a physician to administer a cMet inhibitor to the subject. In certain embodiments, the subject is a Caucasian subject.

In yet another embodiment, the method of the present invention further comprises detecting and quantifying the expression level and/or activation level of HGF/SF protein, the ligand for cMet. In particular embodiments, determining whether the cMet inhibitor should be administered alone in step (d) comprises determining whether expression levels and/or activation levels of cMet, HER3, and HGF/SF proteins in the sample are within the range of medium to high compared to the control protein and/or control sample. In some instances, an expression/activation profile of a subject's tumor sample described as having medium to high levels of cMet, HER3, and HGF/SF can be predictive of a positive response to cMet inhibitor therapy for the subject. In certain embodiments, the subject is a Caucasian subject.

In further embodiments, the subject with a malignancy involving aberrant cMet signaling possesses tumor tissue with one or a plurality of KRAS mutations. Non-limiting examples of KRAS mutations include G12C, G12D, G13D, G12R, G12V, and combinations thereof. In particular instances, step (d) comprises determining that the cMet inhibitor should be administered alone when the KRAS mutation is present in the sample and the expression level and/or activation level of cMet protein, HER3 protein, and HGF/SF protein in the sample is each independently determined to range from medium to high compared to the control protein and/or control sample. In such instances, an expression/activation profile of a subject's tumor sample described as having a KRAS mutation and medium to high levels of cMet, HER3, and HGF/SF can be predictive of a positive response to cMet inhibitor therapy for the subject. In certain embodiments, the subject is a Caucasian subject.

In certain embodiments, determining whether to administer the cMet inhibitor alone to the subject in step (d) comprises determining whether the expression level of cMet protein is within the range of low to medium, and the activation (e.g., phosphorylation) level of cMet proteins is high, both compared to the control protein and/or control sample. In certain other embodiments, determining whether to administer the cMet inhibitor alone to the subject in step (d) further comprises determining whether the expression level of HER3 protein is within the range of low to medium, and the activation (e.g., phosphorylation) level of HER3 protein is high, compared to the control protein and/or control sample. In such instances, an expression/activation profile of a subject's tumor sample described as having a low to medium expression level of cMet protein, alone or in combination with a low to medium expression level of HER3 protein, and a high activation level of cMet protein, alone or in combination with a high activation level of HER3 protein, can be predictive of a positive response to cMet inhibitor therapy for the subject. In certain embodiments, the subject is a Caucasian subject. As described above, in preferred embodiments, the expression levels and/or activation levels of the analytes may be determined using a proximity assay such as CEER and expressed in RFU values, which can be described as being “low,” “medium,” or “high.”

In other embodiments, the methods of the present invention further comprise detecting and/or quantifying the expression level and/or activation level of a truncated cMet protein in a sample obtained from the subject. In particular instances, step (d) comprises determining that the cMet inhibitor should be administered alone when the expression level of the truncated cMet protein in the sample is detectable and the expression level of HER3 protein in the sample is determined to range from medium to high compared to the control protein and/or control sample. In certain embodiments, the subject is a Caucasian subject.

In yet other embodiments, the methods of the present invention further comprise detecting and/or quantifying the expression level and/or activation level of truncated HER3 protein in a sample obtained from the subject. In particular instances, step (d) comprises determining that the cMet inhibitor should be administered alone when the expression level of the truncated HER3 protein in the sample is detectable and the expression level of cMet protein in the sample is determined to range from medium to high compared to the control protein and/or control sample. In certain embodiments, the subject is a Caucasian subject.

In additional embodiments, the methods of the present invention further comprise detecting and/or quantifying the expression level and/or activation level of PI3K protein in a sample obtained from the subject. In particular instances, step (d) comprises determining that the cMet inhibitor should be administered alone when PI3K protein is activated in the sample and the expression level and/or activation level of cMet protein and HGF/SF protein in the sample is each independently determined to range from medium to high compared to the control protein and/or control sample. In certain embodiments, the subject is a Caucasian subject.

In other embodiments, the methods of the present invention further comprise genotyping the subject for an EGFR mutation. Non-limiting examples of EGFR mutations include deletions in exon 19, insertions in exon 20, L858R, G719S, G719A, G719C, L861Q, S768I, T790M, and combinations thereof. In particular embodiments, step (d) comprises determining that the cMet inhibitor should be administered in combination with a pathway-directed therapy when the EGFR mutation is present and when the expression level of cMet protein in the sample is determined to range from medium to high compared to the control protein and/or control sample. In preferred embodiments, the pathway-directed therapy is an EGFR inhibitor. Examples of EGFR inhibitors include, but are not limited to, Cetaximab, Panitumumab, Matuzumab, Nimotuzumab, ErbB1 vaccine, Erlotinib, Gefitinib, ARRY-334543, AEE788, BIBW 2992, EKB 569, CL-387-785, CUDC-101, AV-412, and combinations thereof. In certain embodiments, the subject is a Caucasian subject.

In yet other embodiments, the methods of the present invention further comprise detecting and/or quantifying the expression level and/or activation level of EGFR protein, HER2 protein, PI3K protein, VEGFR1 protein, VEGFR2 protein, and/or VEGFR3 protein in a sample obtained from the subject. In certain particular embodiments, step (d) comprises determining that the cMet inhibitor should be administered in combination with a pathway-directed therapy when the activation level of EGFR protein, HER2 protein, and HER3 protein in the sample is each independently determined to range from medium to high compared to the control protein and/or control sample and the expression level of cMet protein in the sample is determined to range from medium to high compared to the control protein and/or control sample. In other particular embodiments, step (d) comprises determining that the cMet inhibitor should be administered in combination with a pathway-directed therapy when the activation level of PI3K protein in the sample is determined to range from medium to high compared to the control protein and/or control sample and the expression level of HER2 protein, HER3 protein, and cMet protein in the sample is each independently determined to range from medium to high compared to the control protein and/or control sample. In yet other particular embodiments, step (d) comprises determining that the cMet inhibitor should be administered in combination with a pathway-directed therapy when the expression level and/or activation level of cMet protein, EGFR protein, and HER2 protein in the sample is each independently determined to range from medium to high compared to the control protein and/or control sample. In certain embodiments, the subject is a Caucasian subject.

In preferred embodiments, the pathway-directed therapy is an EGFR inhibitor, a pan-HER inhibitor, or combinations thereof. Non-limiting examples of EGFR inhibitors include Cetaximab, Panitumumab, Matuzumab, Nimotuzumab, ErbB1 vaccine, Erlotinib, Gefitinib, ARRY-334543, AEE788, BIBW 2992, EKB 569, CL-387-785, CUDC-101, AV-412, and combinations thereof. Non-limiting examples of pan-HER inhibitors include PF-00299804, neratinib (HKI-272), AC480 (BMS-599626), BMS-690154, PF-02341066, HM781-36B, CI-1033, BIBW-2992, and combinations thereof.

In still yet other particular embodiments, step (d) comprises determining that the cMet inhibitor should be administered in combination with a pathway-directed therapy when the expression level and/or activation level of cMet protein, HER3 protein, and any one, two, or all three of VEGFR1-3 proteins in the sample is each independently determined to range from medium to high compared to the control protein and/or control sample. In preferred embodiments, the pathway-directed therapy is an agent which can alter the expression and/or activation of signaling pathway components, such as, but not limited to, VEGFR inhibitors. Examples of VEGFR inhibitors include, but are not limited to, Bevacizumab (Avastin), HuMV833, VEGF-Trap, AZD 2171, AMG-706, Sunitinib (SU11248), Sorafenib (BAY43-9006), AE-941 (Neovastat), Vatalanib (PTK787/ZK222584), and combinations thereof. In certain embodiments, the subject is a Caucasian subject.

In further embodiments, step (d) comprises determining that a cMet inhibitor should not be administered to the subject when the expression level and/or activation level of cMet protein and/or HER3 protein is each independently low or undetectable and both the level of expression (e.g., total) and activation (e.g., phosphorylation) of EGFR (HER1) protein are high compared to the control protein and/or control sample. In these particular embodiments, the method can further comprise determining that an EGFR inhibitor should be administered when a subject's tumor sample exhibits such an expression/activation profile. Non-limiting examples of EGFR inhibitors include Cetaximab, Panitumumab, Matuzumab, Nimotuzumab, ErbB1 vaccine, Erlotinib, Gefitinib, ARRY-334543, AEE788, BIBW 2992, EKB 569, CL-387-785, CUDC-101, AV-412, and combinations thereof. In certain embodiments, the subject is an Asian subject.

As described herein, the expression levels and/or activation levels of the analytes may be detected and quantified with a proximity dual detection assay such as a Collaborative Enzyme Enhanced Reactive ImmunoAssay (CEER). In particular embodiments, the detected and quantified expression levels and/or activation levels of the analytes may be expressed in RFU values which can be described, e.g., as being “low,” “medium,” or “high.”

In another aspect, the present invention provides a method for monitoring the status of a malignancy involving aberrant cMet signaling in a subject or monitoring how a patient with the malignancy is responding to a therapy, the method comprising:
(a) detecting and/or quantifying serial changes to the expression level and/or activation level of cMet protein in a sample taken from the subject;
(b) detecting and/or quantifying serial changes to the expression level and/or activation level of HER3 protein in the sample; and
(c) comparing the expression level and/or activation level of cMet protein and/or HER3 protein in the sample to (i) the expression level and/or activation level of a control protein over time and/or (ii) the expression level and/or activation level of cMet protein and/or HER3 protein in a control sample over time,
wherein an increasing expression level and/or activation level of cMet protein and/or HER3 protein over time indicates disease progression or a negative response to the therapy, and
wherein a decreasing expression level and/or activation level of cMet protein and/or HER3 protein over time indicates disease remission or a positive response to the therapy.

In certain embodiments, the present invention comprises a method to monitor the status of a malignant cancer in a patient wherein the patient may or may not be receiving anticancer therapy. In specific aspects, the method comprises detecting and quantifying the expression and/or activation level of both cMet and HER3 proteins over time in tumor tissue samples taken from a patient with a malignancy involving aberrant cMet signaling. In certain instances, serial tumor tissue samples from a patient assayed with the methods of the present invention are evaluated by a clinician to monitor the changes in the patient's tumor. In certain instances, when the expression and/or activation levels of cMet protein and/or HER3 protein increase over time, the expression/activation profile can indicate disease progression or a negative response to the anticancer therapy. Disease progression typically corresponds to the appearance of additional signs or symptoms of disease (e.g., cancer). A negative response to therapy describes a situation wherein a patient receiving treatment experiences disease progression or a worsening of symptoms of disease. In other instances, when the expression and/or activation levels of cMet protein and/or HER3 protein decrease over time, the expression/activation profile can indicate disease regression or a positive response to the anticancer therapy. Disease remission or a positive response to therapy typically correlates with an improvement in a patient's disease state. It can indicate that the specific anticancer therapy is successful at alleviating signs or symptoms of the disease.

In certain embodiments, the expression or activation level of a particular analyte of interest may correspond to a level of expression or activation that is compared to a negative control such as an IgG control (i.e., control protein), compared to a standard curve generated for the analyte of interest, compared to a positive control such as a pan-CK control (i.e., control protein), compared to an expression or activation level determined in the presence of an anticancer drug (i.e., control sample), and/or compared to an expression or activation level determined in the absence of an anticancer drug (i.e., control sample). In certain aspects, the control sample can be a cell line or a tissue sample free from a malignancy involving aberrant cMet signaling.

In some embodiments, the expression (e.g., total) level and/or activation (e.g., phosphorylation) level of the one or more analytes is considered to be “changed” in the presence of a therapy such as an anticancer drug when it is at least about 5%, 10%, 15%, 20%, 25%, 30%, 35%, 40%, 45%, 50%, 55%, 60%, 65%, 70%, 75%, 80%, 85%, 90%, or 95% more or less activated than in the absence of the therapy (e.g., anticancer drug). In other embodiments, the expression (e.g., total) level and/or activation (e.g., phosphorylation) level of the one or more analytes is considered to be “substantially decreased” in the presence of a therapy such as an anticancer drug when it is at least about 50%, 55%, 60%, 65%, 70%, 75%, 80%, 85%, 90%, or 95% less activated than in the absence of the therapy. In yet other embodiments, the expression (e.g., total) level and/or activation (e.g., phosphorylation) level of the one or more analytes is considered to be “substantially decreased” in the presence of a therapy such as an anticancer drug (1) when there is a change from “high” or “medium to high” expression and/or activation of the analyte without the therapy to “low,” “weak,” or “detectable” expression and/or activation of the analyte with the therapy, or (2) when there is a change from “medium” expression and/or activation of the analyte without the therapy to “low,” “weak,” or “very weak” expression and/or activation of the analyte with the therapy.

In other embodiments, the expression level and/or activation level of the one or more analytes is expressed as a relative fluorescence unit (RFU) value that corresponds to the signal intensity for a particular analyte of interest that is determined using, e.g., a proximity assay such as the Collaborative Enzyme Enhanced Reactive ImmunoAssay (CEER) described herein. In yet other embodiments, the expression level and/or activation level of the one or more analytes is quantitated by calibrating or normalizing the RFU value that is determined using, e.g., a proximity assay such as CEER, against a standard curve generated for the particular analyte of interest. In certain instances, the RFU value can be calculated based upon a standard curve.

In yet other embodiments, the expression level and/or activation level of the one or more analytes is expressed as a relative fluorescence unit (RFU) value that corresponds to the signal intensity for a particular analyte of interest that is determined using, e.g., a proximity assay such as CEER described herein. In other embodiments, the expression level and/or activation level of the one or more analytes is expressed as “low,” “medium,” or “high” that corresponds to increasing signal intensity for a particular analyte of interest that is determined using, e.g., a proximity assay such as CEER. In some instances, an undetectable or minimally detectable level of expression or activation of a particular analyte of interest that is determined using, e.g., a proximity assay such as CEER, may be expressed as “undetectable.” In other instances, a low level of expression or activation of a particular analyte of interest that is determined using, e.g., a proximity assay such as CEER, may be expressed as “low.” In yet other instances, a moderate level of expression or activation of a particular analyte of interest that is determined using, e.g., a proximity assay such as CEER, may be expressed as “medium.” In still yet other instances, a moderate to high level of expression or activation of a particular analyte of interest that is determined using, e.g., a proximity assay such as CEER, may be expressed as “medium to high.” In further instances, a very high level of expression or activation of a particular analyte of interest that is determined using, e.g., a proximity assay such as CEER, may be expressed as “high.”

In certain embodiments, the expression or activation level of a particular analyte of interest, when expressed as “low,” “medium,” or “high,” may correspond to a level of expression or activation that is at least about 0; 5,000; 10,000; 15,000; 20;000; 25,000; 30,000; 35,000; 40,000; 45,000; 50,000; 60,000; 70;000; 80,000; 90,000; 100,000 RFU, e.g., when compared to a negative control such as an IgG control, when compared to a standard curve generated for the analyte of interest, when compared to a positive control such as a pan-CK control, when compared to an expression or activation level determined in the presence of an anticancer drug, and/or when compared to an expression or activation level determined in the absence of an anticancer drug. In some instances, the correlation is analyte-specific. As a non-limiting example, a “low” level of expression or activation determined using, e.g., a proximity assay such as CEER, may correspond 10,000 RFUs in expression or activation for one analyte and a 50,000 RFUs for another analyte when compared to a reference expression or activation level.

In certain embodiments, the expression or activation level of a particular analyte of interest may correspond to a level of expression or activation referred to as “low,” “medium,” or “high” that is relative to a reference expression level or activation level, e.g., when compared to a negative control such as an IgG control, when compared to a standard curve generated for the analyte of interest, when compared to a positive control such as a pan-CK control, when compared to an expression or activation level determined in the presence of an anticancer drug, and/or when compared to an expression or activation level determined in the absence of an anticancer drug. In some instances, the correlation is analyte-specific. As a non-limiting example, a “low” level of expression or activation determined using, e.g., a proximity assay such as CEER, may correspond to a 2-fold increase in expression or activation for one analyte and a 5-fold increase for another analyte when compared to a reference expression or activation level.

In particular embodiments, the therapy comprises treatment with a cMet inhibitor. In some embodiments, the cMet inhibitor is selected from a group consisting of a multi-kinase inhibitor, a tyrosine kinase inhibitor, and a monoclonal antibody. In particular aspects, a multi-kinase inhibitor (e.g., pan-HER inhibitor) is an agent that blocks a plurality of kinases, such as, but not limited to, cMet, RON, EGFR, HER2, HER3, VEGFR1, VEGR2, VEGFR3, PI3K, SHC, p95HER2, IGF-1R, and c-Kit. In other aspects, a tyrosine kinase inhibitor is an agent that blocks one or more tyrosine kinases such as, but not limited to, cMet, RON, EGFR, HER2, HER3, VEGFR1, VEGR2, and/or VEGFR3. Non-limiting examples of small molecule tyrosine kinase inhibitors which can be used in the present invention include a gefitinib (Iressa®), erlotinib (Tarceva®), pelitinib, CP-654577, CP-724714, canertinib (CI 1033), HKI-272, lapatinib (GW-572016; Tykerb®), PKI-166, AEE788, BMS-599626, HKI-357, BIBW 2992, ARRY-334543, JNJ-26483327, and JNJ-26483327; and combinations thereof. Non-limiting examples of monoclonal antibodies for use in the present invention include trastuzumab (Herceptin®), pertuzumab (2C4), alemtuzumab (Campath®), bevacizumab (Avastin®), cetuximab (Erbitux®), gemtuzumab (Mylotarg®), panitumumab (Vectibix™), rituximab (Rituxan®), and tositumomab (BEXXAR®), and combinations thereof. Non-limiting examples of pan-HER inhibitors, HER2 inhibitors, EGFR inhibitors and VEGFR inhibitors are described above.

Non-limiting examples of compounds that modulate cMet activity are described herein and include monoclonal antibodies, small molecule inhibitors, and combinations thereof. In preferred embodiments, the cMet-modulating compound inhibits cMet activity and/or blocks cMet signaling, e.g., is a cMet inhibitor. Examples of cMet inhibitors include, but are not limited to, monoclonal antibodies such as AV299, L2G7, AMG102, DN30, OA-5D5, and MetMAb; and small molecule inhibitors of cMet such as ARQ197, AMG458, BMS-777607, XL 184, XL880, INCB28060, E7050, GSK1363089/XL880, K252a, LY2801653, MP470, MGCD265, MK-2461, NK2, NK4, SGX523, SU11274, SU5416, PF-04217903, PF-02341066, PHA-665752, JNJ-38877605; and combinations thereof.

In preferred embodiments, the subject has a malignant cancer involving aberrant cMet signaling. In certain instances, the malignancy involving aberrant cMet signaling is a carcinoma of the breast, liver, lung, gastric, ovary, kidney, thyroid, or combinations thereof. In certain other instances, the malignancy involving aberrant cMet signaling is non-small cell lung cancer (NSCLC).

In certain instances, the present invention provides a method for monitoring the status of a malignancy in a patient or monitoring how a patient with such a malignancy is responding the therapy. In particular instances, the status of the disease in the patient can correspond to disease remission or a positive response to therapy, wherein the symptoms of the disease (e.g., cancer) are evaluated and correlated to clinically-defined patient outcomes (e.g., clinical outcomes). Non-limiting examples of clinical outcomes include response rate (RR), complete response (CR), partial response (PR), stable disease (SD), time to progression (TTP), progression free survival (PFS), and overall survival (OS). The response rate includes the percentage of patients with positive responses, such as tumor shrinkage or disappearance, to a defined therapy for the treatment of a disease. Complete response includes a clinical endpoint described by the disappearance of all signs of cancer in response to treatment after a period of time. For example, if at the end of the time or treatment course, there is no residual disease that can be identified by measurements of symptom control and quality of life as performed by examination, X-ray and scan, or analysis of biomarkers of the disease, the patient is described herein to exhibit complete response to therapy. In certain instances, complete response is the disappearance of all tumor lesions (see, National Cancer Institute's RECIST, updated in January 2009). On the other hand, partial response includes a clinical endpoint describing the disappearance of some, but not all, signs of cancer in response to treatment after a period of time. For example, if at the end of the time or treatment course, there is some detectable residual disease that can be identified by measurements of symptom control and quality of life as performed by examination, X-ray and scan, or analysis of biomarkers of the disease, the patient is described herein to exhibit partial response to therapy. In certain instances, partial response includes a 30% decrease in the sum of the longest diameter of the tumor lesions (see, National Cancer Institute's RECIST, updated in January 2009). Stable disease includes a clinical endpoint in cancer characterized by the appearance of no new tumors and no substantial change in the size of existing, known tumors. According to RECIST, stable disease is defined as small changes that do not meet the criteria of complete response, partial response, and progressive disease (which is defined as a 20% increase in the sum of the longest diameter of the tumor lesions). The time to progression (TTP) includes the measure of time after a disease is diagnosed or treated until the disease starts to worsen (e.g., appearance of new tumors, increase in tumor size; change in the quality of life, or change in symptom control). Progression free survival (PFS) includes the length of time during and after a treatment of a disease in which a patient is living with the disease without additional symptoms of the disease. Overall survival (OS) includes the clinical endpoint describing patients who are alive for a defined period of time after being diagnosed with or treated for a disease, such as cancer.

In some embodiments, the pathway-directed therapy comprises an agent that interferes with the function of activated signal transduction pathway components in cancer cells. Non-limiting examples of such agents include those listed below in Table 1.

TABLE 1

| EGFR (ErbB1) (A) | HER-2 (ErbB2) (C) | HER-3 (ErbB3) (E) | HER-4 (ErbB4) target |
|---|---|---|---|
| Cetuximab | Trastuzumab | Antibody (U3) | |
| Panitumumab | (Herceptin ®) | | |
| Matuzumab | Pertuzumab (2C4) | | |
| Nimotuzumab | BMS-599626* | | |
| ErbB1 vaccine | | | |
| | *Heterodimerization | | |
| | HER-1/2; Phase 1 | | |

| EGFR (ErbB1) (B) | HER-2 (ErbB2) (D) | ErbB1/2 (F) | ErbB1/2/4 (G) |
|---|---|---|---|
| Erlotinib | CP-724714 (Pfizer) | Lapatinib (Tykerb ®) | Canertinib* |
| Gefitinib | | HKI-272* | ARRY-334543 |
| EKB 569* | | HKI-357 (Preclinical) | JNJ-26483327 |
| CL-387-785** | | BIBW 2992** | JNJ-26483327 |
| *(Wyeth, Irreversible, | | *Wyeth, Irreversible, I/II | *Pfizer, |
| II CRC) | | NSCLC, Breast | Irreversible, |
| **(Wyeth, Irreversible, | | **Boehringer | II NSCLC, Breast |
| Preclinical) | | Ingelheim, Irreversible, | |
| | | I/II Prostate, Ovarian, | |
| | | Breast | |

| Raf (H) | SRC (H) | Mek: (I) | NFkB-IkB (I) |
|---|---|---|---|
| Sorafenib | AZ | PD-325901 (II: NSCLC) | |
| PLX4032 (Plexxikon) | | AZD6244 - Array/Az | |
| | | XL518 Exelisis/DNA | |

| | | VEGFR2 and | |
|---|---|---|---|
| mTor (J) | PI3K (J) | VEGFR1 (K) | VEGFR1/2/3: |
| Rad 001: Everolimus* | PX-866* | Avastin (DNA) | AZD 2171 (NSCLC, |
| Temsirolimus** | | HuMV833* | CRC) |
| AP-23573*** | | VEGF-Trap** | AMG-706 (+PDGFR) |
| *Everolimus (Novartis, | *P110alpha specific | *(PDL) anti-VEGFa | |
| combination with | inhibition; ProIX | **Regeneron/Aventis | |
| Gefetinib/Erlotinib; I/II: | Pharma; Preclinical | (Receptor mimic) | |
| NSCLC, Glioblastoma) | NSCLC | (Phase 2) | |
| **Temsirolimus (Wyeth, | | | |
| combination with | | | |
| Gefetinib/Erlotinib; I/II: | | | |
| NSCLC, Glioblastoma) | | | |
| ***AP-23573 (Ariad, I/II: | | | |
| Endometrial) | | | |

| | |
|---|---|
| VEGFR2 target (L) | EPH A-D |

| | | |
|---|---|---|
| DC101* | CDP-791 (UCB) | Bay-579352 (+PDGFR) |
| IMC-IC11** | CP-547632* | ABT-869* |
| IMC1121B Fully | AG13736** | BMS-540215 (+FGFR1) |
| humanized | E-7080 (Eisai) | KRN-951 |
| CDP-791*** | CHIR-258*** | BBIW |
| Pazopanib**** | OSI-930 (+cKit, PDGFR) | |
| *Imclone (Phase 2/3?) | *OSI, PFIZER: (+ErbB1 + | *(+CSF1R, Erk, Flt-3, |
| **Chimeric IgG1 against | PDGFR) (NSCLC, Ovarian | PDGFR) |
| VEGFR2 | Phase 2) | |
| ***Celltech, pegalated | **Pfizer: VEGFR1,2 and | |
| di-Fab antibody against | PDGFRbeta) (RCC II) | |
| R2 | ***(VEGFR1,2 | |
| ****GSK, Multiple | FGFR3, PDGFR) | |
| myeloma, ovarian, RCC | | |
| Phase 3 enrollment | | |
| completed, sarcoma II) | | |

| VEGFR 2/ErbB1/2 | VEGFR2/3/Raf/ | | VEGFR2/1/3, |
|---|---|---|---|
| (ErbB1)/cMet/ | PDGFR/cKit/ | | Flt-3, cFMS, |
| FGFR (M) | Flt-3 (N) | TIE 1/2 | PDGFR/cKit (O) |
| ZD6474* | Sorafenib* | | PTK787 (Not cFMS, |
| XL647** | | | FLT-3) |
| AEE 788*** | | | Sunitinib |
| | | | XL-999 |
| | | | SU-6668 (Pfizer) |
| | | | GSK |
| | | | AZ (AZD2171) |
| | | | BMS |
| | | | Novartis (AEE-788) |
| | | | Amgen |
| | | | Others |
| *(vandetanib) (Phase | *(RCC, HCC, | | |
| III: thyroid, NSCLC) | NSCLC(III), | | |
| **(Exelixis; Also | Melanoma(III)) | | |
| EPHB2): (Patient | | | |
| resistant to Erlotinib; | | | |
| Asian patients) (Phase 2) | | | |
| ***(Novartis, Phase1/2) | | | |

| PDGFR target (P) | Abl target: (Q) | FTL 3 | RET |
|---|---|---|---|
| Tandutinib | Imatinib | | |
| Nilotinib | Dasatinib | | |
| | Nilotinib | | |
| | AT-9283 | | |
| | AZD-0530 | | |
| | Bosutinib | | |

| Kit target (R) | HGFR1/2 | FGFR1-4 | IGF-1R Target (S) |
|---|---|---|---|
| AMG-706 | | Chiron | Merck |
| XL-880 | | | Pfizer |
| XL-999 | | | Novartis |

| HSP90 inhibitors: | Anti-Mitotic Drugs: | Other targets: |
|---|---|---|
| IPI-504* | Docetaxel* | HDAC inhibitors |
| 17-AAG** | Paclitaxel** | BCL2 |
| | Vinblastine, Vincristine, | Chemotherapeutics |
| | Vinorelbine*** | (breakdown) |
| | | Proteosome inhibitors |
| *(Infinity Pharma, | *(Microtubule stabilizer; | |
| Mutant ErbB1, I/II | Adjuvant and advanced | |
| multiple myeloma, | Breast cancer; NSCLC, | |
| GIST) | Androgen independent | |
| **(Kosan, I/II solid | Prostate cancer) | |
| tumors) | **(Microtubule stabilizer; | |
| | Adjuvant and advanced | |
| | Breast cancer; NSCLC, | |
| | Ovarian cancer, AIDS | |
| | related Kaposi sarcoma) | |
| | ***(Microtubule De- | |
| | stabilizers) | |

In certain embodiments, the pathway-directed therapy comprises an anti-signaling agent (i.e., a cytostatic drug) such as a monoclonal antibody or a tyrosine kinase inhibitor; an anti-proliferative agent; a chemotherapeutic agent (i.e., a cytotoxic drug); a hormonal therapeutic agent; a radiotherapeutic agent; a vaccine; and/or any other compound with the ability to reduce or abrogate the uncontrolled growth of aberrant cells such as cancerous cells. In some embodiments, the subject is treated with one or more anti-signaling agents, anti-proliferative agents, and/or hormonal therapeutic agents in combination with at least one chemotherapeutic agent.

Examples of anti-signaling agents suitable for use in the present invention include, without limitation, monoclonal antibodies such as trastuzumab (Herceptin®), pertuzumab (2C4), alemtuzumab (Campath®), bevacizumab (Avastin®), cetuximab (Erbitux®), gemtuzumab (Mylotarg®), panitumumab (Vectibix™), rituximab (Rituxan®), tositumomab (BEXXAR®), AV299, L2G7, AMG102, DN30, OA-5D5, and MetMAb; tyrosine kinase inhibitors such as gefitinib (Iressa®), sunitinib (Sutent®), erlotinib (Tarceva®), lapatinib (GW-572016; Tykerb®), canertinib (CI 1033), semaxinib (SU5416), vatalanib (PTK787/ZK222584), sorafenib (BAY 43-9006; Nexavar®), imatinib mesylate (Gleevec®), leflunomide (SU101), vandetanib (ZACTIMA™; ZD6474), pelitinib, CI 1033, CL-387-785, CP-654577, CP-724714, CUDC-101, HKI-272, HKI-357, PKI-166, ARQ197, AMG458, AEE788, BMS-599626, BMS-690154, HKI-357, BIBW 2992, EKB 569, HM781-36B, ARRY-380, ARRY-334543, AV-412, BMS-777607, XL 184, XL880, INCB28060, E7050, GSK1363089/XL880, K252a, LY2801653, MP470, MGCD265, MK-2461, NK2, NK4, SGX523, SU11274, SU5416, PF-04217903, JNJ-38877605PHA-665752, PF-00299804, PF-02341066, JNJ-26483327, and JNJ-26483327; and combinations thereof.

Exemplary anti-proliferative agents include mTOR inhibitors such as sirolimus (rapamycin), temsirolimus (CCI-779), everolimus (RAD001), BEZ235, and XL765; AKT inhibitors such as 1L6-hydroxymethyl-chiro-inositol-2-(R)-2-O-methyl-3-O-octadecyl-sn-glycerocarbonate, 9-methoxy-2-methylellipticinium acetate, 1,3-dihydro-1-(1-((4-(6-phenyl-1H-imidazo[4,5-g]quinoxalin-7-yl)phenyl)methyl)-4-piperidinyl)-2H-benzimidazol-2-one, 10-(4′-(N-diethylamino)butyl)-2-chlorophenoxazine, 3-formylchromone thiosemicarbazone (Cu(II)Cl₂ complex), API-2, a 15-mer peptide derived from amino acids 10-24 of the proto-oncogene TCL1 (Hiromura et al., J. Biol. Chem., 279:53407-53418 (2004), KP372-1, and the compounds described in Kozikowski et al., J. Am. Chem. Soc., 125:1144-1145 (2003) and Kau et al., Cancer Cell, 4:463-476 (2003); PI3K inhibitors such as PX-866, wortmannin, LY 294002, quercetin, tetrodotoxin citrate, thioperamide maleate, GDC-0941 (957054-30-7), IC87114, PI-103, PIK93, BEZ235 (NVP-BEZ235), TGX-115, ZSTK474, (−)-deguelin, NU 7026, myricetin, tandutinib, GDC-0941 bismesylate, GSK690693, KU-55933, MK-2206, OSU-03012, perifosine, triciribine, XL-147, PIK75, TGX-221, NU 7441, PI 828, XL-765, and WHI-P 154; MEK inhibitors such as PD98059, ARRY-162, RDEA119, U0126, GDC-0973, PD184161, AZD6244, AZD8330, PD0325901, and ARRY-142886; and combinations thereof.

Non-limiting examples of pan-HER inhibitors include PF-00299804, neratinib (HKI-272), AC480 (BMS-599626), BMS-690154, PF-02341066, HM781-36B, CI 1033, BIBW-2992, and combinations thereof.

Non-limiting examples of chemotherapeutic agents include platinum-based drugs (e.g., oxaliplatin, cisplatin, carboplatin, spiroplatin, iproplatin, satraplatin, etc.), alkylating agents (e.g., cyclophosphamide, ifosfamide, chlorambucil, busulfan, melphalan, mechlorethamine, uramustine, thiotepa, nitrosoureas, etc.), anti-metabolites (e.g., 5-fluorouracil, azathioprine, 6-mercaptopurine, methotrexate, leucovorin, capecitabine, cytarabine, floxuridine, fludarabine, gemcitabine (Gemzar®), pemetrexed (ALIMTA®), raltitrexed, etc.), plant alkaloids (e.g., vincristine, vinblastine, vinorelbine, vindesine, podophyllotoxin, paclitaxel (Taxol®), docetaxel (Taxotere®), etc.), topoisomerase inhibitors (e.g., irinotecan, topotecan, amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), pharmaceutically acceptable salts thereof, stereoisomers thereof, derivatives thereof, analogs thereof, and combinations thereof.

Examples of hormonal therapeutic agents include, without limitation, aromatase inhibitors (e.g., aminoglutethimide, anastrozole (Arimidex®), letrozole (Femara®), vorozole, exemestane (Aromasin®), 4-androstene-3,6,17-trione (6-OXO), 1,4,6-androstatrien-3,17-dione (ATD), formestane (Lentaron®), etc.), selective estrogen receptor modulators (e.g., bazedoxifene, clomifene, fulvestrant, lasofoxifene, raloxifene, tamoxifen, toremifene, etc.), steroids (e.g., dexamethasone), finasteride, and gonadotropin-releasing hormone agonists (GnRH) such as goserelin, pharmaceutically acceptable salts thereof, stereoisomers thereof, derivatives thereof, analogs thereof, and combinations thereof.

Non-limiting examples of cancer vaccines useful in the present invention include ANYARA from Active Biotech, DCVax-LB from Northwest Biotherapeutics, EP-2101 from IDM Pharma, GV1001 from Pharmexa, IO-2055 from Idera Pharmaceuticals, INGN 225 from Introgen Therapeutics and Stimuvax from Biomira/Merck.

Examples of radiotherapeutic agents include, but are not limited to, radionuclides such as ⁴⁷Sc, ⁶⁴Cu, ⁶⁷Cu, ⁸⁹Sr, ⁸⁶Y, ⁸⁷Y, ⁹⁰Y, ¹⁰⁵Rh, ¹¹¹Ag, ¹¹¹In, ^{117m}Sn, ¹⁴⁹Pm, ¹⁵³Sm, ¹⁶⁶Ho, ¹⁷⁷Lu, ¹⁸⁶Re, ¹⁸⁸Re, ²¹¹At, and ²¹²Bi, optionally conjugated to antibodies directed against tumor antigens.

Non-limiting examples of compounds that modulate HER2 activity are described herein and include monoclonal antibodies, tyrosine kinase inhibitors, and combinations thereof. In preferred embodiments, the HER2-modulating compound inhibits HER2 activity and/or blocks HER2 signaling, e.g., is a HER2 inhibitor. Examples of HER2 inhibitors include, but are not limited to, monoclonal antibodies such as trastuzumab (Herceptin®) and pertuzumab (2C4); small molecule tyrosine kinase inhibitors such as gefitinib (Iressa®), erlotinib (Tarceva®), pelitinib, CP-654577, CP-724714, canertinib (CI 1033), HKI-272, lapatinib (GW-572016; Tykerb®), PKI-166, AEE788, BMS-599626, HKI-357, BIBW 2992, ARRY-380, ARRY-334543, CUDC-101, JNJ-26483327, and JNJ-26483327; and combinations thereof. In other embodiments, the HER2-modulating compound activates the HER2 pathway, e.g., is a HER2 activator.

Non-limiting examples of compounds that modulate cMet activity are described herein and include monoclonal antibodies, small molecule inhibitors, and combinations thereof. In preferred embodiments, the cMet-modulating compound inhibits cMet activity and/or blocks cMet signaling, e.g., is a cMet inhibitor. Examples of cMet inhibitors include, but are not limited to, monoclonal antibodies such as AV299, L2G7, AMG102, DN30, OA-5D5, and MetMAb; small molecule inhibitors of cMet such as ARQ197, AMG458, BMS-777607, XL 184, XL880, INCB28060, E7050, GSK1363089/XL880, K252a, LY2801653, MP470, MGCD265, MK-2461, NK2, NK4, SGX523, SU11274, SU5416, PF-04217903, PF-02341066, PHA-665752, and JNJ-38877605; and combinations thereof.

In certain embodiments, the reference expression or activation level of the one or more analytes (e.g., one or more HER3 and/or cMet signaling pathway components) determined in step (c) is obtained from a normal cell such as a non-cancerous cell from a healthy individual not having a cancer such as non-small cell lung cancer. In certain other embodiments, the reference expression or activation level of the one or more analytes (e.g., one or more HER3 and/or cMet signaling pathway components) determined in step (c) is obtained from a tumor cell such as a non-small cell lung cancer cell from a sample from a patient with a cancer such as non-small cell lung cancer.

In some embodiments, the reference expression or activation level of the one or more analytes (e.g., one or more HER3 and/or cMet signaling pathway components) determined in step (c) is obtained from a cell (e.g., a tumor cell such as a malignant cancer cell obtained from a patient sample) that is not treated with the anticancer drug. In particular embodiments, the cell that is not treated with the anticancer drug is obtained from the same sample that the isolated cell (e.g., a test cell to be interrogated) used to produce the cellular extract is obtained. In certain instances, the presence of a lower level of expression or activation of the one or more analytes (e.g., one or more HER3 and/or cMet signaling pathway components) compared to the reference expression or activation level indicates that the anticancer drug is suitable for the treatment of the malignant cancer involving aberrant cMet signaling (e.g., the malignant tumor has an increased likelihood of response to the anticancer drug). In certain other instances, the presence of an identical, similar, or higher level of expression or activation of the one or more analytes (e.g., one or more HER3 and/or cMet signaling pathway components) compared to the reference expression or activation level indicates that the anticancer drug is unsuitable for the treatment of the malignant cancer involving aberrant cMet signaling (e.g., the malignant tumor has a decreased likelihood of response to the anticancer drug).

In alternative embodiments, the reference expression or activation level of the one or more analytes (e.g., one or more HER3 and/or cMet signaling pathway components) determined in step (c) is obtained from a cell sensitive to the anticancer drug that is treated with the anticancer drug. In such embodiments, the presence of an identical, similar, or lower level of expression or activation of the one or more analytes (e.g., one or more HER3 and/or cMet signaling pathway components) compared to the reference expression or activation level indicates that the anticancer drug is suitable for the treatment of the malignant cancer involving aberrant cMet signaling (e.g., the malignant tumor has an increased likelihood of response to the anticancer drug). In certain other alternative embodiments, the reference expression or activation level of the one or more analytes (e.g., one or more HER3 and/or cMet signaling pathway components) determined in step (c) is obtained from a cell resistant to the anticancer drug that is treated with the anticancer drug. In such embodiments, the presence of an identical, similar, or higher level of expression or activation of the one or more analytes (e.g., one or more HER3 and/or cMet signaling pathway components) compared to the reference expression or activation level indicates that the anticancer drug is unsuitable for the treatment of the malignant cancer involving aberrant cMet signaling (e.g., the malignant tumor has a decreased likelihood of response to the anticancer drug).

In certain embodiments, a higher level of expression or activation of the one or more analytes (e.g., one or more HER3 and/or cMet signaling pathway components) detected and quantified in steps (a) and (b) is considered to be present in a sample (e.g., a cellular extract) when the expression or activation level is at least about 1.5, 2, 2.5, 3, 3.5, 4, 4.5, 5, 5.5, 6, 6.5, 7, 7.5, 8, 8.5, 9, 9.5, 10, 15, 20, 25, 30, 35, 40, 45, 50, or 100-fold higher (e.g., about 1.5-3, 2-3, 2-4, 2-5, 2-10, 2-20, 2-50, 3-5, 3-10, 3-20, 3-50, 4-5, 4-10, 4-20, 4-50, 5-10, 5-15, 5-20, or 5-50-fold higher) than the reference expression or activation level of the corresponding analyte in a cell (e.g., a malignant cancer cell obtained from a patient sample) not treated with the anticancer drug, in an anticancer drug-sensitive cell treated with the anticancer drug, or in an anticancer drug-resistant cell treated with the anticancer drug.

In other embodiments, a lower level of expression or activation of the one or more analytes (e.g., one or more HER3 and/or cMet signaling pathway components) detected and quantified in steps (a) and (b) is considered to be present in a sample (e.g., a cellular extract) when the expression or activation level is at least about 1.5, 2, 2.5, 3, 3.5, 4, 4.5, 5, 5.5, 6, 6.5, 7, 7.5, 8, 8.5, 9, 9.5, 10, 15, 20, 25, 30, 35, 40, 45, 50, or 100-fold lower (e.g., about 1.5-3, 2-3, 2-4, 2-5, 2-10, 2-20, 2-50, 3-5, 3-10, 3-20, 3-50, 4-5, 4-10, 4-20, 4-50, 5-10, 5-15, 5-20, or 5-50-fold lower) than the reference expression or activation level of the corresponding analyte in a cell (e.g., a malignant cancer cell obtained from a patient sample) not treated with the anticancer drug, in an anticancer drug-sensitive cell treated with the anticancer drug, or in an anticancer drug-resistant cell treated with the anticancer drug.

Non-limiting examples of signal transduction molecules and pathways that may be interrogated using the present invention include those shown in Table 2.

TABLE 2

| Pathway | ErbB1 | ErbB1 | ErbB1 | ErbB1 | ErbB1-PI3K | PTEN | | |
|---|---|---|---|---|---|---|---|---|
| 1 | | Phospho | She | ubiquitin | | | | |
| Pathway | ErbB1 | ErbB1VIII | ErbB1VIII | ErbB1VIII | ErbB1VIII | ErbB1VIII | PTEN | |
| 2 | | | Phospho | Shc | ubiquitin | PI3K | | |
| Pathway | ErbB2 | ErbB2 | HER-2 Shc | ErbB2: | ErbB2 | PTEN | | |
| 3 | | Phospho | | PI3K | ubiquitin | | | |
| | | | | Complex | | | | |
| Pathway | ErbB2 | P95Truncated | ErbB2Phospho | P95Truncated | HER-2 Shc | ERBB2: | ErbB2 | P95ErbB2: |
| 4 | | ErbB2 | | ERBB2 | | PI3K | ubiquitin | PI3K |
| | | | | Phospho | | Complex | | |
| Pathway | ErbB3 | ErbB3 | ErbB3:PI3K | ErbB3 PI3K | ErbB3:Shc | | | |
| 5 | | Phospho | Complex | Phospho | | | | |
| Pathway | ErbB4 | ErbB4 | ErbB4:Shc | | | | | |
| 6 | | Phospho | | | | | | |
| Pathway | IGF-1R | IGF- | IGF-1R:IRS | IRS:PI3K | Phospho IRS | IGF-1R: | | |
| 7 | | IRPhospho | | | | PI3K | | |
| Pathway | INSR | INSRPhospho | | | | | | |
| 8 | | | | | | | | |
| Pathway | KIT | KIT Phospho | | | | | | |
| 9 | | | | | | | | |
| Pathway | FLT3 | FLT3Phospho | | | | | | |
| 10 | | | | | | | | |
| Pathway | HGFR 1 | HGFR 1 | | | | | | |
| 11 | | Phospho | | | | | | |
| Pathway | HGFR 2 | HGFR 2 | | | | | | |
| 12 | | Phospho | | | | | | |
| Pathway | RET | RET Phospho | | | | | | |
| 13 | | | | | | | | |
| Pathway | PDGFR alpha | PDGFR alpha | | | | | | |
| 14 | | Phospho | | | | | | |
| Pathway | PDGFR beta | PDGFR beta | | | | | | |
| 15 | | Phospho | | | | | | |
| Pathway | VEGFR 1 | VEGFR 1 | VEGFR 1: | VEGFR 1: | | | | |
| 16 | | Phospho | PLCγcomplex | Src | | | | |
| Pathway | VEGFR 2 | VEGFR 2 | VEGFR 2: | VEGFR 2: | VEGFR- | VEGFR-2, | | |
| 17 | | Phospho | PLCγ | Src | 2/heparin | VE-cadherin | | |
| | | | complex | | sulphate | complex | | |
| | | | | | complex | | | |
| Pathway | VEGFR 3 | VEGFR 3 | | | | | | |
| 18 | | Phospho | | | | | | |
| Pathway | FGFR 1 | FGFR 1 | | | | | | |
| 19 | | Phospho | | | | | | |
| Pathway | FGFR 2 | FGFR 2 | | | | | | |
| 20 | | Phospho | | | | | | |
| Pathway | FGFR 3 | FGFR 3 | | | | | | |
| 21 | | Phospho | | | | | | |
| Pathway | FGFR 4 | FGFR 4 | | | | | | |
| 22 | | Phospho | | | | | | |
| Pathway | TIE 1 | TIE 1 Phospho | | | | | | |
| 23 | | | | | | | | |
| Pathway | TIE 2 | TIE 2 | | | | | | |
| 24 | | Phospho | | | | | | |
| Pathway | EPHA | EPHA | | | | | | |
| 25 | | Phospho | | | | | | |
| Pathway | EPHB | EPHB | | | | | | |
| 26 | | Phospho | | | | | | |
| Pathway | NFkB-IkB | phospho-IKB | Total NFKB | Total P65 | | | | |
| 27 | complex | (S32) | Phospho | IkBa | | | | |
| | | Total IkB | NFκB(S536) | Phospho | | | | |
| | | | | P65 IkBa | | | | |
| Pathway | ER | Phospho ER | ER-AIB1 | Other ER | | | | |
| 28 | | | | complexes | | | | |
| Pathway | PR | Phospho Pr | | PR | | | | |
| 29 | | | | complexes | | | | |
| Pathway | Hedgehog | | | | | | | |
| 30 | Pathway | | | | | | | |
| Pathway | Wnt pathway | | | | | | | |
| 31 | | | | | | | | |
| Pathway | Notch | | | | | | | |
| 32 | Pathway | | | | | | | |
| Pathway | Total Mek | Total Erk | Total Rsk-1 | Total Stat3 | Phospho Bad | Total Fak | Total cSrc | Total Ras |
| 33 | Phospho Mek | Phospho Erk | Phospho Rsk-1 | Phospho Stat- | (S112) | Phospho Fak | Phospho | Phospho |
| | (S217/S221) | (T202/Y204) | (T357/5363) | 3 (Y705) | Bad (total) | (Y576) | cSrc(Y416) | Ras |
| | | | | (S727) | | | | |
| | | | | Total Stat 1 | | | | |
| | | | | Phospho | | | | |
| | | | | Stat1 (Y 701) | | | | |
| Pathway | Akt (Total) | Phospho Akt | Phospho Bad | Phospho Bad | Bad:14-3-3 | Total mTor | Total | GSK3beta |
| 34 | Phospho Akt | (T308) | (S112) | (S136) | complex | Phospho mTor | p7056K | Total |
| | (T473) | | Bad (total) | | | (S2448) | Phospho | (Phospho Ser 9) |
| | | | | | | | p70S6K | |
| | | | | | | | (T229) | |
| | | | | | | | (T389) | |
| Pathway | Total Jnk | Total P38 | Total Rb | Total p53 | phospho- | Total c-Jun | Total | |
| 35 | Phospho Jnk | Phospho P38 | Phospho Rb | Phospho p53 | CREB(S133) | Phospho-c-Jun; | Paxillin | |
| | (T183/Y185) | (T180/Y182) | (S249/T252) | (S392) | Total | (S63) | Phospho | |
| | | | Phospho Rb | Phospho p53 | CREB | | Paxillin | |
| | | | (S780) | (S20) | | | (Y118) | |
| Pathway | Ki67 | Cleaved | TOPO2 | | | | | |
| 36 | | Caspase 3,8,9 | | | | | | |
| | | others | | | | | | |
| Pathway | TGFbeta | | | | | | | |
| 37 | | | | | | | | |

Non-limiting examples of analytes such as signal transduction molecules that can be interrogated for expression (e.g., total amount) levels and/or activation (e.g., phosphorylation) levels in a sample such as a cellular extract include receptor tyrosine kinases, non-receptor tyrosine kinases, tyrosine kinase signaling cascade components, nuclear hormone receptors, nuclear receptor coactivators, nuclear receptor repressors, and combinations thereof.

In one embodiment, the methods of the present invention comprise determining the expression (e.g., total amount) level and/or activation (e.g., phosphorylation) level of one of the following analytes in a cellular extract: (1) HER1/EGFR/ErbB1; (2) HER2/ErbB2; (3) p95HER2; (4) HER3/ErbB3; (5) cMet; (6) truncated cMet; (7) HGF/SF; (8) PI3K (e.g., PIK3CA and/or PIK3R1); (9) Shc; (10) Akt; (11) p70S6K; (12) VEGFR (e.g., VEGFR1, VEGFR2, and/or VEGFR3); and (13) truncated HER3.

In another embodiment, the present invention comprises determining the expression (e.g., total) level and/or activation (e.g., phosphorylation) level of one of the following pairs of two analytes in a cellular extract, wherein “1”=HER1, “2”=HER2, “3”=p95HER2, “4”=HER3, “5”=cMet, “6”=truncated cMet, “7”=HGF/SF, “8”=PI3K (e.g., PIK3CA and/or PIK3R1), “9”=Shc, “10”=Akt, “11”=p70S6K, “12”=VEGFR (e.g., VEGFR1, 2, and/or 3), and “13”=truncated HER3: 1,2; 1,3; 1,4; 1,5; 1,6; 1,7; 1,8; 1,9; 1,10; 1,11; 1,12; 1,13; 2,3; 2,4; 2,5; 2,6; 2,7; 2,8; 2,9; 2,10; 2,11; 2,12; 2,13; 3,4; 3,5; 3,6; 3,7; 3,8; 3,9; 3,10; 3,11; 3,12; 3,13; 4,5; 4,6; 4,7; 4,8; 4,9; 4,10; 4,11; 4,12; 4,13; 5,6; 5,7; 5,8; 5,9; 5,10; 5,11; 5,12; 5,13; 6,7; 6,8; 6,9; 6,10; 6,11; 6,12; 6,13; 7,8; 7,9; 7,10; 7,11; 7,12; 7,13; 8,9; 8,10; 8,11; 8,12; 8,13; 9,10; 9,11; 9,12; 9,13; 10,11; 10,12; 10,13; 11,12; 11,13; and 12,13.

In yet another embodiment, the present invention comprises determining the expression (e.g., total) level and/or activation (e.g., phosphorylation) level of one of the following sets of three analytes in a cellular extract, wherein “1”=HER1, “2”=HER2, “3”=p95HER2, “4”=HER3, “5”=cMet, “6”=truncated cMet, “7”=HGF/SF, “8”=PI3K (e.g., PIK3CA and/or PIK3R1), “9”=Shc, “10”=Akt, “11”=p70S6K, “12”=VEGFR (e.g., VEGFR1, 2, and/or 3), and “13”=truncated HER3: 1,2,3; 1,2,4; 1,2,5; 1,2,6; 1,2,7; 1,2,8; 1,2,9; 1,2,10; 1,2,11; 1,2,12; 1,2,13; 1,3,4; 1,3,5; 1,3,6; 1,3,7; 1,3,8; 1,3,9; 1,3,10; 1,3,11; 1,3,12; 1,3,13; 1,4,5; 1,4,6; 1,4,7; 1,4,8; 1,4,9; 1,4,10; 1,4,11; 1,4,12; 1,4,13; 1,5,6; 1,5,7; 1,5,8; 1,5,9; 1,5,10; 1,5,11; 1,5,12; 1,5,13; 1,6,7; 1,6,8; 1,6,9; 1,6,10; 1,6,11; 1,6,12; 1,6,13; 1,7,8; 1,7,9; 1,7,10; 1,7,11; 1,7,12; 1,7,13; 1,8,9; 1,8,10; 1,8,11; 1,8,12; 1,8,13; 1,9,10; 1,9,11; 1,9,12; 1,9,13; 1,10,11; 1,10,12; 1,10,13; 1,11,12; 1,11,13; 1,12,13, 2,3,4; 2,3,5; 2,3,6; 2,3,7; 2,3,8; 2,3,9; 2,3,10; 2,3,11; 2,3,12; 2,3,13; 2,4,5; 2,4,6; 2,4,7; 2,4,8; 2,4,9; 2,4,10; 2,4,11; 2,4,12; 2,4,13; 2,5,6; 2,5,7; 2,5,8; 2,5,9; 2,5,10; 2,5,11; 2,5,12; 2,5,13; 2,6,7; 2,6,8; 2,6,9; 2,6,10; 2,6,11; 2,6,12; 2,6,13; 2,7,8; 2,7,9; 2,7,10; 2,7,11; 2,7,12; 2,7,13; 2,8,9; 2,8,10; 2,8,11; 2,8,12; 2,8,13; 2,9,10; 2,9,11; 2,9,12; 2,9,13; 2,10,11; 2,10,12; 2,10,13; 2,11,12; 2,11,13; 2,12,13; 3,4,5; 3,4,6; 3,4,7; 3,4,8; 3,4,9; 3,4,10; 3,4,11; 3,4,12; 3,4,13; 3,5,6; 3,5,7; 3,5,8; 3,5,9; 3,5,10; 3,5,11; 3,5,12; 3,5,13; 3,6,7; 3,6,8; 3,6,9; 3,6,10; 3,6,11; 3,6,12; 3,6,13; 3,7,8; 3,7,9; 3,7,10; 3,7,11; 3,7,12; 3,7,13; 3,8,9; 3,8,10; 3,8,11; 3,8,12; 3,8,13; 3,9,10; 3,9,11; 3,9,12; 3,9,13; 3,10,11; 3,10,12; 3,10,13; 3,11,12; 3,11,13; 3,12,13; 4,5,6; 4,5,7; 4,5,8; 4,5,9; 4,5,10; 4,5,11; 4,5,12; 4,5,13; 4,6,7; 4,6,8; 4,6,9; 4,6,10; 4,6,11; 4,6,12; 4,6,13; 4,7,8; 4,7,9; 4,7,10; 4,7,11; 4,7,12; 4,7,13; 4,8,9; 4,8,10; 4,8,11; 4,8,12; 4,8,13; 4,9,10; 4,9,11; 4,9,12; 4,9,13; 4,10,11; 4,10,12; 4,10,13; 4,11,12; 4,11,13; 4,12,13; 5,6,7; 5,6,8; 5,6,9; 5,6,10; 5,6,11; 5,6,12; 5,6,13; 5,7,8; 5,7,9; 5,7,10; 5,7,11; 5,7,12; 5,7,13; 5,8,9; 5,8,10; 5,8,11; 5,8,12; 5,8,13; 5,9,10; 5,9,11; 5,9,12; 5,9,13; 5,10,11; 5,10,12; 5,10,13; 5,11,12; 5,11,13; 5,12,13, 6,7,8; 6,7,9; 6,7,10; 6,7,11; 6,7,12; 6,7,13; 6,8,9; 6,8,10; 6,8,11; 6,8,12; 6,8,13; 6,9,10; 6,9,11; 6,9,12; 6,9,13; 6,10,11; 6,10,12; 6,10,13; 6,11,12; 6,11,13; 6,12,13; 7,8,9; 7,8,10; 7,8,11; 7,8,12; 7,8,13; 7,9,10; 7,9,11; 7,9,12; 7,9,13; 7,10,11; 7,10,12; 7,10,13; 7,11,12; 7,11,13; 7,12,13; 8,9,10; 8,9,11; 8,9,12; 8,9,13; 8,10,11; 8,10,12; 8,10,13; 8,11,12; 8,11,13; 8,12,13; 9,10,11; 9,10,12; 9,10,13; 9,11,12; 9,11,13; 9,12,13; 10,11,12; 10,11,13; 10,12,13; and 11,12,13.

In still yet another embodiment, the present invention comprises determining the expression (e.g., total) level and/or activation (e.g., phosphorylation) level of one of the following sets of four analytes in a cellular extract, wherein “1”=HER1, “2”=HER2, “3”=p95HER2, “4”=HER3, “5”=cMet, “6”=truncated cMet, “7”=HGF/SF, “8”=PI3K (e.g., PIK3CA and/or PIK3R1), “9”=Shc, “10”=Akt, “11”=p70S6K, “12”=VEGFR (e.g., VEGFR1, 2, and/or 3), and “13”=truncated HER3: 1,2,3,4; 1,2,3,5; 1,2,3,6; 1,2,3,7; 1,2,3,8; 1,2,3,9; 1,2,3,10; 1,2,3,11; 1,2,3,12; 1,2,3,13; 1,3,4,5; 1,3,4,6; 1,3,4,7; 1,3,4,8; 1,3,4,9; 1,3,4,10; 1,3,4,11; 1,3,4,12; 1,3,4,13; 1,4,5,6; 1,4,5,7; 1,4,5,8; 1,4,5,9; 1,4,5,10; 1,4,5,11; 1,4,5,12; 1,4,5,13; 1,5,6,7; 1,5,6,8; 1,5,6,9; 1,5,6,10; 1,5,6,11; 1,5,6,12; 1,5,6,13; 1,6,7,8; 1,6,7,9; 1,6,7,10; 1,6,7,11; 1,6,7,12; 1,6,7,13; 1,7,8,9; 1,7,8,10; 1,7,8,11; 1,7,8,12; 1,7,8,13; 1,8,9,10; 1,8,9,11; 1,8,9,12; 1,8,9,13; 1,9,10,11; 1,9,10,12; 1,9,10,13; 1,10,11,12; 1,10,11,13; 1,11,12,13; 2,3,4,5; 2,3,4,6; 2,3,4,7; 2,3,4,8; 2,3,4,9; 2,3,4,10; 2,3,4,11; 2,3,4,12; 2,3,4,13; 2,4,5,6; 2,4,5,7; 2,4,5,8; 2,4,5,9; 2,4,5,10; 2,4,5,11; 2,4,5,12; 2,4,5,13; 2,5,6,7; 2,5,6,8; 2,5,6,9; 2,5,6,10; 2,5,6,11; 2,5,6,12; 2,5,6,13; 2,6,7,8; 2,6,7,9; 2,6,7,10; 2,6,7,11; 2,6,7,12; 2,6,7,13; 2,7,8,9; 2,7,8,10; 2,7,8,11; 2,7,8,12; 2,7,8,13; 2,8,9,10; 2,8,9,11; 2,8,9,12; 2,8,9,13; 2,9,10,11; 2,9,10,12; 2,9,10,13; 2,10,11,12; 2,10,11,13; 2,11,12,13; 3,4,5,6; 3,4,5,7; 3,4,5,8; 3,4,5,9; 3,4,5,10; 3,4,5,11; 3,4,5,12; 3,4,5,13; 3,5,6,7; 3,5,6,8; 3,5,6,9; 3,5,6,10; 3,5,6,11; 3,5,6,12; 3,5,6,13; 3,6,7,8; 3,6,7,9; 3,6,7,10; 3,6,7,11; 3,6,7,12; 3,6,7,13; 3,7,8,9; 3,7,8,10; 3,7,8,11; 3,7,8,12; 3,7,8,13; 3,8,9,10; 3,8,9,11; 3,8,9,12; 3,8,9,13; 3,9,10,11; 3,9,10,12; 3,9,10,13; 3,10,11,12; 3,10,11,13; 3,11,12,13; 4,5,6,7; 4,5,6,8; 4,5,6,9; 4,5,6,10; 4,5,6,11; 4,5,6,12; 4,5,6,13; 4,6,7,8; 4,6,7,9; 4,6,7,10; 4,6,7,11; 4,6,7,12; 4,6,7,13; 4,7,8,9; 4,7,8,10; 4,7,8,11; 4,7,8,12; 4,7,8,13; 4,8,9,10; 4,8,9,11; 4,8,9,12; 4,8,9,13; 4,9,10,11; 4,9,10,12; 4,9,10,13; 4,10,11,12; 4,10,11,13; 4,11,12,13; 5,6,7,8; 5,6,7,9; 5,6,7,10; 5,6,7,11; 5,6,7,12; 5,6,7,13; 5,7,8,9; 5,7,8,10; 5,7,8,11; 5,7,8,12; 5,7,8,13; 5,8,9,10; 5,8,9,11; 5,8,9,12; 5,8,9,13; 5,9,10,11; 5,9,10,12; 5,9,10,13; 5,10,11,12; 5,10,11,13; 5,11,12,13; 6,7,8,9; 6,7,8,10; 6,7,8,11; 6,7,8,12; 6,7,8,13; 6,8,9,10; 6,8,9,11; 6,8,9,12; 6,8,9,13; 6,9,10,11; 6,9,10,12; 6,9,10,13; 6,10,11,12; 6,10,11,13; 6,11,12,13; 7,8,9,10; 7,8,9,11; 7,8,9,12; 7,8,9,13; 7,9,10,11; 7,9,10,12; 7,9,10,13; 7,10,11,12; 7,10,11,13; 7,11,12,13; 8,9,10,11; 8,9,10,12, 8,9,10,13; 8,10,11,12; 8,10,11,13; 8,11,12,13; 9,10,11,12; 9,10,11,13; 9,11,12,13; and 10,11,12,13.

In another embodiment, the present invention comprises determining the expression (e.g., total) level and/or activation (e.g., phosphorylation) level of any possible combination of five of the following analytes: “1”=HER1, “2”=HER2, “3”=p95HER2, “4”=HER3, “5”=cMet, “6”=truncated cMet, “7”=HGF/SF, “8”=PI3K (e.g., PIK3CA and/or PIK3R1), “9”=Shc, “10”=Akt, “11”=p70S6K, “12”=VEGFR (e.g., VEGFR1, 2, and/or 3), and “13”=truncated HER3. As non-limiting examples, the combination of five analytes may comprise one of the following: 1,2,3,4,5; 2,3,4,5,6; 3,4,5,6,7; 4,5,6,7,8; 5,6,7,8,9; 6,7,8,9,10; 7,8,9,10,11; 8,9,10,11,12; or 9,10,11,12,13.

In yet another embodiment, the present invention comprises determining the expression (e.g., total) level and/or activation (e.g., phosphorylation) level of any possible combination of six of the following analytes: “1”=HER1, “2”=HER2, “3”=p95HER2, “4”=HER3, “5”=cMet, “6”=truncated cMet, “7”=HGF/SF, “8”=PI3K (e.g., PIK3CA and/or PIK3R1), “9”=Shc, “10”=Akt, “11”=p70S6K, “12”=VEGFR (e.g., VEGFR1, 2, and/or 3), and “13”=truncated HER3. As non-limiting examples, the combination of six analytes may comprise one of the following: 1,2,3,4,5,6; 2,3,4,5,6,7; 3,4,5,6,7,8; 4,5,6,7,8,9; 5,6,7,8,9,10; 6,7,8,9,10,11; 7,8,9,10,11,12; or 8,9,10,11,12,13.

In still yet another embodiment, the present invention comprises determining the expression (e.g., total) level and/or activation (e.g., phosphorylation) level of any possible combination of seven of the following analytes: “1”=HER1, “2”=HER2, “3”=p95HER2, “4”=HER3, “5”=cMet, “6”=truncated cMet, “7”=HGF/SF, “8”=PI3K (e.g., PIK3CA and/or PIK3R1), “9”=Shc, “10”=Akt, “11”=p70S6K, “12”=VEGFR (e.g., VEGFR1, 2, and/or 3), and “13”=truncated HER3. As non-limiting examples, the combination of seven analytes may comprise one of the following: 1,2,3,4,5,6,7; 2,3,4,5,6,7,8; 3,4,5,6,7,8,9; 4,5,6,7,8,9,10; 5,6,7,8,9,10,11; 6,7,8,9,10,11,12; or 7,8,9,10,11,12,13.

In another embodiment, the present invention comprises determining the expression (e.g., total) level and/or activation (e.g., phosphorylation) level of any possible combination of eight of the following analytes: “1”=HER1, “2”=HER2, “3”=p95HER2, “4”=HER3, “5”=cMet, “6”=truncated cMet, “7”=HGF/SF, “8”=PI3K (e.g., PIK3CA and/or PIK3R1), “9”=Shc, “10”=Akt, “11”=p70S6K, “12”=VEGFR (e.g., VEGFR1, 2, and/or 3), and “13”=truncated HER3. As non-limiting examples, the combination of eight analytes may comprise one of the following: 1,2,3,4,5,6,7,8; 2,3,4,5,6,7,8,9; 3,4,5,6,7,8,9,10; 4,5,6,7,8,9,10,11; 5,6,7,8,9,10,11,12; or 6,7,8,9,10,11,12,13.

In yet another embodiment, the present invention comprises determining the expression (e.g., total) level and/or activation (e.g., phosphorylation) level of any possible combination of nine of the following analytes: “1”=HER1, “2”=HER2, “3”=p95HER2, “4”=HER3, “5”=cMet, “6”=truncated cMet, “7”=HGF/SF, “8”=PI3K (e.g., PIK3CA and/or PIK3R1), “9”=Shc, “10”=Akt, “11”=p70S6K, “12”=VEGFR (e.g., VEGFR1, 2, and/or 3), and “13”=truncated HER3. As non-limiting examples, the combination of nine analytes may comprise one of the following: 1,2,3,4,5,6,7,8,9; 2,3,4,5,6,7,8,9,10; 3,4,5,6,7,8,9,10,11; 4,5,6,7,8,9,10,11,12; or 5,6,7,8,9,10,11,12,13.

In still yet another embodiment, the present invention comprises determining the expression (e.g., total) level and/or activation (e.g., phosphorylation) level of any possible combination of ten of the following analytes: “1”=HER1, “2”=HER2, “3”=p95HER2, “4”=HER3, “5”=cMet, “6”=truncated cMet, “7”=HGF/SF, “8”=PI3K (e.g., PIK3CA and/or PIK3R1), “9”=Shc, “10”=Akt, “11”=p70S6K, “12”=VEGFR (e.g., VEGFR1, 2, and/or 3), and “13”=truncated HER3. As non-limiting examples, the combination of ten analytes may comprise one of the following: 1,2,3,4,5,6,7,8,9,10; 2,3,4,5,6,7,8,9,10,11; 3,4,5,6,7,8,9,10,11,12; or 4,5,6,7,8,9,10,11,12,13.

In another embodiment, the present invention comprises determining the expression (e.g., total) level and/or activation (e.g., phosphorylation) level of any possible combination of eleven of the following analytes: “1”=HER1, “2”=HER2, “3”=p95HER2, “4”=HER3, “5”=cMet, “6”=truncated cMet, “7”=cKit, “8”=PI3K (e.g., PIK3CA and/or PIK3R1), “9”=Shc, “10”=Akt, “11”=p70S6K, “12”=VEGFR (e.g., VEGFR1, 2, and/or 3), and “13”=truncated HER3. As non-limiting examples, the combination of eleven analytes may comprise one of the following: 1,2,3,4,5,6,7,8,9,10,11; 2,3,4,5,6,7,8,9,10,11,12; or 3,4,5,6,7,8,9,10,11,12,13.

In yet another embodiment, the present invention comprises determining the expression (e.g., total) level and/or activation (e.g., phosphorylation) level of any possible combination of twelve of the following analytes: “1”=HER1, “2”=HER2, “3”=p95HER2, “4”=HER3, “5”=cMet, “6”=truncated cMet, “7”=HGF/SF, “8”=PI3K (e.g., PIK3CA and/or PIK3R1), “9”=Shc, “10”=Akt, “11”=p70S6K, “12”=VEGFR (e.g., VEGFR1, 2, and/or 3), and “13”=truncated HER3. As non-limiting examples, the combination of twelve analytes may comprise one of the following: 1,2,3,4,5,6,7,8,9,10,11,12; or 2,3,4,5,6,7,8,9,10,11,12,13.

In still yet another embodiment, the present invention comprises determining the expression (e.g., total) level and/or activation (e.g., phosphorylation) level of all thirteen of the following analytes: “1”=HER1, “2”=HER2, “3”=p95HER2, “4”=HER3, “5”=cMet, “6”=truncated cMet, “7”=HGF/SF, “8”=PI3K (e.g., PIK3CA and/or PIK3R1), “9”=Shc, “10”=Akt, “11”=p70S6K, “12”=VEGFR (e.g., VEGFR1, 2, and/or 3), and “13”=truncated HER3.

In one particular embodiment, the present invention comprises determining the expression (e.g., total) level and/or activation (e.g., phosphorylation) level of HER1, HER2, p95HER2, HER3, cMet, truncated cMet, and/or truncated HER3. In another particular embodiment, the present invention comprises determining the expression (e.g., total) level and/or activation (e.g., phosphorylation) level of HER1, HER2, HER3, cMet, IGF1R, HGF/SF, PI3K (e.g., PIK3CA and/or PIK3R1), Shc, truncated cMet, and/or truncated HER3. In yet another particular embodiment, the present invention comprises determining the expression (e.g., total) level and/or activation (e.g., phosphorylation) level of HER1, HER2, p95HER2, HER3, cMet, IGF1R, HGF/SF, PI3K (e.g., PIK3CA and/or PIK3R1), Shc, Aid, p70S6K, VEGFR (e.g., VEGFR1, 2, and/or 3), truncated cMet, and/or truncated HER3.

In certain embodiments, the present invention further comprises determining the expression (e.g., total) level and/or activation (e.g., phosphorylation) level of one or more (e.g., at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or more) additional analytes in the cellular extract. In some embodiments, the one or more (e.g., at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or more) additional analytes comprises one or more signal transduction molecules selected from the group consisting of receptor tyrosine kinases, non-receptor tyrosine kinases, tyrosine kinase signaling cascade components, nuclear hormone receptors, nuclear receptor coactivators, nuclear receptor repressors, and combinations thereof.

In particular embodiments, the present invention further comprises determining the expression (e.g., total) level and/or activation (e.g., phosphorylation) level of one or any combination of 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or more of the following additional analytes in a cellular extract: HER4, MEK, PTEN, SGK3, 4E-BP1, ERK2 (MAPK1), ERK1 (MAPK3), PDK1, PDK2, GSK-3β, Raf, SRC, NFkB-IkB, mTOR, EPH-A, EPH-B, EPH-C, EPH-D, FLT-3, TIE-1, TIE-2, c-FMS, Abl, FTL 3, RET, FGFR1, FGFR2, FGFR3, FGFR4, ER, PR, NCOR, AIB1, RON, PIP2, PIP3, p27, protein tyrosine phosphatases (e.g., PTP1B, PTPN13, BDP1, etc.), receptor dimers, other HER3 signaling pathway components, other cMet signaling pathway components, and combinations thereof.

### IV. c-Met Mediated Cancers

c-Met can be overexpressed in many malignancies. In c-Met mediated cancers, amplification and/or activation mutations within the tyrosine kinase domain, juxtamembrane domain, or semaphorin domain have been identified. Selecting a suitable anticancer drug for the treatment of a c-Met mediated cancer is possible by assessing the level of expression and/or activation state of c-Met in the presence of therapeutics. Activation of c-Met leads to increased cell growth, invasion, angiogenesis, and metastasis. In certain embodiments, the present invention provides methods of selecting appropriate therapeutic strategies to inhibit c-Met activation and/or overexpression.

In one embodiment, the present invention provides a method for selecting a suitable anticancer drug for the treatment of a c-Met mediated cancer (e.g., a malignancy involving aberrant c-Met signaling), the method comprising:
(a) determining the expression level and/or activation level of c-Met and optionally one or more additional analytes in a cellular extract produced from an isolated cancer cell; and
(b) selecting a suitable anticancer drug for the treatment of the c-Met mediated cancer based upon the expression level and/or activation level of the one or more analytes determined in step (a).

In some instances, the present invention provides a method for selecting a suitable anticancer drug for the treatment of a c-Met mediated cancer (e.g., a malignancy involving aberrant c-Met signaling), the method comprising:
(a) isolating a cancer cell after administration of an anticancer drug, or prior to incubation with an anticancer drug;
(b) lysing the isolated cell to produce a cellular extract;
(c) determining the expression level and/or activation level of c-Met and optionally one or more additional analytes in the cellular extract; and
(d) comparing the expression level and/or activation level of c-Met and optionally one or more additional analytes determined in step (c) to a reference expression and/or activation profile of c-Met and optionally one or more additional analytes that is generated in the absence of the anticancer drug to determine whether the anticancer drug is suitable or unsuitable for the treatment of the c-Met mediated cancer.

In another embodiment, the present invention provides a method for identifying the response of a c-Met mediated cancer (e.g., a malignancy involving aberrant c-Met signaling) to treatment with an anticancer drug, the method comprising:
(a) determining the expression level and/or activation level of c-Met and optionally one or more additional analytes in a cellular extract produced from an isolated cancer cell; and
(b) identifying the response of the c-Met mediated cancer to treatment with an anticancer drug based upon the expression level and/or activation level of the one or more analytes determined in step (a).

In some instances, the present invention provides a method for identifying the response of a c-Met mediated cancer (e.g., a malignancy involving aberrant c-Met signaling) to treatment with an anticancer drug, the method comprising:
(a) isolating a cancer cell after administration of an anticancer drug, or prior to incubation with an anticancer drug;
(b) lysing the isolated cell to produce a cellular extract;
(c) determining the expression level and/or activation level of c-Met and optionally one or more additional analytes in the cellular extract; and
(d) comparing the expression level and/or activation level of c-Met and optionally one or more additional analytes determined in step (c) to a reference expression and/or activation profile of c-Met and optionally one or more additional analytes that is generated in the absence of the anticancer drug to identify whether the c-Met mediated cancer is responsive or non-responsive to treatment with the anticancer drug.

In yet another embodiment, the present invention provides a method for predicting the response of a subject having a c-Met mediated cancer (e.g., a malignancy involving aberrant c-Met signaling) to treatment with an anticancer drug, the method comprising:
(a) determining the expression level and/or activation level of c-Met and optionally one or more additional analytes in a cellular extract produced from an isolated cancer cell; and
(b) predicting the response of the subject having the c-Met mediated cancer to treatment with an anticancer drug based upon the expression level and/or activation level of the one or more analytes determined in step (a).

In some instances, the present invention provides a method for predicting the response of a subject having a c-Met mediated cancer (e.g., a malignancy involving aberrant c-Met signaling) to treatment with an anticancer drug, the method comprising:
(a) isolating a cancer cell after administration of an anticancer drug, or prior to incubation with an anticancer drug;
(b) lysing the isolated cell to produce a cellular extract;
(c) determining the expression level and/or activation level of c-Met and optionally one or more additional analytes in the cellular extract; and
(d) comparing the expression level and/or activation level of c-Met and optionally one or more additional analytes determined in step (c) to a reference expression and/or activation profile of c-Met and optionally one or more additional analytes that is generated in the absence of the anticancer drug to predict the likelihood that the subject having the c-Met mediated cancer will respond to treatment with the anticancer drug.

In a further embodiment, the present invention provides a method for monitoring the status of a c-Met mediated cancer (e.g., a malignancy involving aberrant c-Met signaling) in a subject or monitoring how a patient with the c-Met mediated cancer is responding to therapy, the method comprising:
(a) determining the expression level and/or activation level of c-Met and optionally one or more additional analytes over time in cellular extracts produced from an isolated cancer cell to detect and quantify serial changes to the expression level and/or activation level of cMet protein; and
(b) monitoring the status of the c-Met mediated cancer or how a patient with the c-Met mediated cancer is responding to therapy based upon the expression level and/or activation level of the one or more analytes determined in step (a) over time.

In some embodiments, step (b) comprises comparing the expression level and/or activation level of c-Met and optionally one or more additional analytes determined in step (a) over time to a reference expression and/or activation profile of c-Met and optionally one or more additional analytes over time to monitor the status of the c-Met mediated cancer or how a patient with the c-Met mediated cancer is responding to therapy, wherein increasing expression and/or activation levels of cMet protein and optionally one or more additional analytes determined in step (a) over time indicate disease progression or a negative response to the therapy, and wherein decreasing expression and/or activation levels of cMet protein and optionally one or more additional analytes determined in step (a) over time indicate disease remission or a positive response to the therapy.

In certain aspects, the present invention provides methods to evaluate c-Met mediated cancer pathways in patient samples such as tumor tissue, circulating tumor cells (CTC), or fine needle aspirates (FNA). The methods herein provide an optimum therapeutic strategy for the patient. In one aspect, at least one, two, three, four, five, six, seven, eight, nine, ten, or more of the following additional analytes can be screened or interrogated to determine the response to a c-Met mediated cancer therapy (e.g., a c-Met inhibitor): HER1, HER2, p95HER2, HER3, truncated HER3, IGF1R, cKit, PI3K (e.g., PIK3CA, PIK3R1), Shc, Akt (e.g., Akt1, Akt2, Akt3), p70S6K, VEGFR (e.g., VEGFR1, VEGFR2, VEGFR3), PDGFR (e.g., PDGFRA, PDGFRB), RON, and combinations thereof. For example, a responder to XL-880 has activated c-MET and VEGFR2, while a non-responder may have a combination of RTKs activated.

In certain other instances, the methods provided herein find utility in selecting a combination therapy for the treatment of a malignant cancer involving aberrant c-Met signaling. For example, cancer patients with activated c-MET, VEGFR2, and EGFR can be successfully treated with a combination of Iressa® and XL-880, while cancer patients with activated c-MET, VEGFR2, HER1, HER2, p95HER2, and HER3 can be treated with Tykerb®+XL-880.

In tumor cells, it is believed that c-Met activation causes the triggering of a diverse series of signaling cascades resulting in cell growth, proliferation, invasion, and protection from apoptosis. Data from cellular and animal tumor models suggest that the underlying biological mechanisms for tumorgenicity of c-Met mediated cancers are typically achieved in three different ways: (1) with the establishment of HGF/c-Met autocrine loops; (2) via c-Met or HGF overexpression; and (3) in the presence of kinase-activating mutations in the c-Met receptor coding sequence. Overexpression of HGF and c-Met is indicative of the increased aggressiveness of tumors and poor prognostic signs in cancer patients. HGF/c-Met signaling induces tumor angiogenesis by inducing proliferation and migration in endothelial cells, by inducing expression of vascular endothelial growth factor (VEGF), a key proangiogenic factor, as well as by dramatically downregulating thrombospondin 1 (TSP-1), a negative regulator of angiogenesis. HGF and c-Met expression have been observed in tumor biopsies of most solid tumors, and c-Met signaling has been documented in a wide range of human malignancies, including stomach (gastric), bladder, breast, cervical, colorectal, gastric, head and neck, liver, lung, ovarian, pancreatic, prostrate, renal, and thyroid cancers, as well as in various sarcomas, hematopoietic malignancies, and melanoma. Most notably, activating mutations in the tyrosine kinase domain of c-Met have been positively identified in patients with a hereditary form of papillary renal cancer, directly implicating c-Met in human tumorigenesis.

In certain embodiments, the present invention provides methods for detecting the expression and activation states of c-Met and optionally a plurality of deregulated signal transducers, in tumor cells derived from tumor tissue or circulating cells of a solid tumor in a specific, multiplex, high-throughput assay. The present invention also provides methods and compositions for the selection of appropriate therapies to down-regulate or shut down one or more deregulated signaling pathways. Thus, embodiments of the invention may be used to facilitate the design of personalized therapies based on the particular molecular signature provided by the collection of activated signal transduction proteins in a given patient's tumor such as a lung tumor (e.g., NSCLC).

In some embodiments, the anticancer drug (e.g., one or more anticancer drugs suitable for the treatment of a c-Met mediated cancer such as non-small cell lung cancer) comprises an anti-signaling agent (i.e., a cytostatic drug) such as a monoclonal antibody or a tyrosine kinase inhibitor; an anti-proliferative agent; a chemotherapeutic agent (i.e., a cytotoxic drug); a hormonal therapeutic agent; a radiotherapeutic agent; a vaccine; and/or any other compound with the ability to reduce or abrogate the uncontrolled growth of aberrant cells such as cancerous cells. In some embodiments, the isolated cells are treated with one or more anti-signaling agents, anti-proliferative agents, and/or hormonal therapeutic agents in combination with at least one chemotherapeutic agent.

In certain embodiments, the antibody such as a HGF- or c-Met-specific antibody prevents ligand/receptor binding, resulting in growth inhibition and tumor regression by inhibiting proliferation and enhancing apoptosis. In some instances, a combination of monoclonal antibodies can also be used. The strategy of using monoclonal antibodies allows for exclusive specificity against HGF/c-Met, a relatively long half-life compared to small molecule kinase inhibitors, and the potential to elicit a host immune response against tumor cells. AMG102 is a fully human IgG2 monoclonal antibody that selectively binds and neutralizes HGF, thereby preventing its binding to c-Met and subsequent activation. AMG102 has been shown to enhance the effects of various standard chemotherapeutic agents such as temozolomide and docetaxel in vitro and in xenografts when combined. MetMAb is a humanized, monovalent, antagonistic anti-c-Met antibody derived from the agonistic monoclonal antibody 5D5. MetMAb binds to c-Met with high affinity and remains on the cell surface with c-Met, preventing HGF binding and subsequent c-Met phosphorylation as well as downstream signaling activity and cellular responses. Recent preclinical studies show that MetMAb is a potent anti-c-Met inhibitor that has promise as a therapeutic antibody in human cancer, especially in combination with EGFR and/or VEGF inhibitors.

Small molecule inhibitors of c-Met include, but are not limited to, ARQ197 (ArQule), which is a non-ATP-competitive agent highly selective for the c-Met receptor. Other selective c-Met inhibitors have recently entered initial clinical evaluations and include: JNJ-38877605 (Johnson & Johnson), which is a small-molecule, ATP-competitive inhibitor of the catalytic activity of c-Met; PF-04217903 (Pfizer), which is an orally available, ATP-competitive small-molecule inhibitor of c-Met with selectivity of >1000-fold for c-Met compared with a screening panel of >150 protein kinases; SGX523 (SGX Pharmaceuticals), which is another highly selective, ATP-competitive inhibitor of c-Met with >1,000-fold selectivity for c-Met over all other kinases in a screening panel of 213 protein kinases and potent antitumor activity when dosed orally in human xenograft models with no overt toxicity.

GSK 1363089/XL880 (Exelixis) is another example of a small molecule inhibitor of c-Met which targets c-Met at an IC50 of 0.4 nM. Binding affinity is high to both c-Met and VEGFR2, causing a conformational change in the kinase to move XL880 deeper into the ATP-binding pocket. The time on target is >24 hours for both receptors. XL880 has good oral bioavailability, and it is a CYP450 substrate, but not an inhibitor or inducer. Two Phase I clinical trials examined different administration schedules of XL880, either on a 5 day on/9 day off schedule (Study 1) or as a fixed daily dose (Study 2). XL880 acts on two cooperating pathways for proliferation and survival at different points in time, already providing a therapeutic solution for tumor response to the initial assault on tumor angiogenesis. Phase II trials have started in multiple tumor types, including papillary renal cancer, gastric cancer, and head and neck cancers.

XL184 (Exelixis) is a novel, orally administered, small molecule anticancer compound that, in preclinical models, has demonstrated potent inhibition of both c-Met and VEGFR2. MP470 (SuperGen) is a novel, orally bioavailable small molecule with inhibitory activity against c-Met as well as several other protein tyrosine kinase targets, including mutant forms of c-Kit, mutant PDGFRa, and mutant Flt-3. MGCD265 (Methylgene) potently inhibits c-Met, Ron, VEGFRs, and Tie-2 enzymatic activities in vitro and has been reported to abrogate HGF dependent cellular endpoints, such as cell scatter and wound healing, as well as VEGF-dependent responses such as in vitro angiogenesis and in vivo vascular permeability. MK-2461 (Merck) is a potent inhibitor of c-Met, KDR, FGFR1/2/3, and Flt 1/3/4 that is especially active in preclinical models with MET gene amplification, in which c-Met is constitutively phosphorylated. MK-2461 has been well tolerated in early Phase I evaluation.

In certain instances, binding of HGF ligand to the c-Met receptor can be inhibited by subregions of HGF or c-Met that can act as decoys or antagonists. These decoys and antagonists stoichiometrically compete with the ligand or receptor without leading to c-Met activation, thereby preventing activation of downstream pathways and biological outcomes. Several HGF and c-Met variants have been validated experimentally as antagonists both in vitro and in vivo and work by blocking ligand binding or preventing c-Met dimerization. In addition, molecular analogs to HGF that have been shown to compete with HGF for c-Met binding have been developed.

### V. Construction of Antibody Arrays

In certain aspects, the expression level and/or activation state of one or more (e.g., a plurality) of analytes (e.g., signal transduction molecules) in a cellular extract of tumor cells such as lung cancer cells is detected using an antibody-based array comprising a dilution series of capture antibodies restrained on a solid support. The arrays typically comprise a plurality of different capture antibodies at a range of capture antibody concentrations that are coupled to the surface of the solid support in different addressable locations.

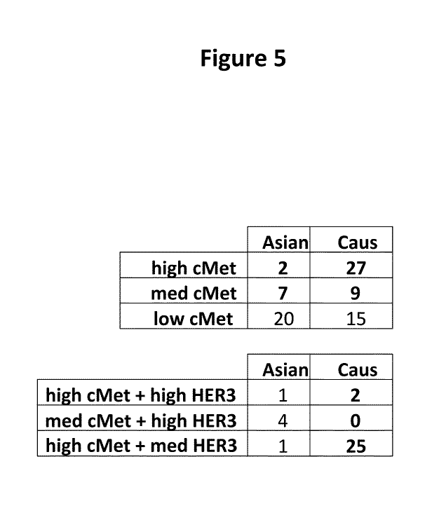

In one particular embodiment, the present invention provides an addressable array having superior dynamic range comprising a plurality of dilution series of capture antibodies restrained on a solid support, in which the capture antibodies in each dilution series are specific for one or more analytes corresponding to a component of a signal transduction pathway and other target proteins. In various aspects, this embodiment includes arrays that comprise components of signal transduction pathways characteristic of particular tumors, e.g., signal transduction pathways active in lung cancer cells (e.g., c-Met pathways). Thus, the present invention may be advantageously practiced wherein each signal transduction molecule or other protein of interest with a potential expression or activation defect causing cancer is represented on a single array or chip. In some aspects, the components of a given signal transduction pathway active in a particular tumor cell are arrayed in a linear sequence that corresponds to the sequence in which information is relayed through a signal transduction pathway within a cell. Examples of such arrays are described herein and also shown in FIGS. 5-9 of PCT Publication No. WO2009/108637, the disclosure of which is herein incorporated by reference in its entirety for all purposes. The capture antibodies specific for one or more components of a given signal transduction pathway active in a particular tumor cell can also be printed in a randomized fashion to minimize any surface-related artifacts.

The solid support can comprise any suitable substrate for immobilizing proteins. Examples of solid supports include, but are not limited to, glass (e.g., a glass slide), plastic, chips, pins, filters, beads, paper, membranes, fiber bundles, gels, metal, ceramics, and the like. Membranes such nylon (Biotrans™, ICN Biomedicals, Inc. (Costa Mesa, Calif.); Zeta-Probe®, Bio-Rad Laboratories (Hercules, Calif.)), nitrocellulose (Protran®, Whatman Inc. (Florham Park, N.J.)), and PVDF (Immobilon™, Millipore Corp. (Billerica, Mass.)) are suitable for use as solid supports in the arrays of the present invention. Preferably, the capture antibodies are restrained on glass slides coated with a nitrocellulose polymer, e.g., FAST® Slides, which are commercially available from Whatman Inc. (Florham Park, N.J.).

Particular aspects of the solid support which are desirable include the ability to bind large amounts of capture antibodies and the ability to bind capture antibodies with minimal denaturation. Another suitable aspect is that the solid support displays minimal “wicking” when antibody solutions containing capture antibodies are applied to the support. A solid support with minimal wicking allows small aliquots of capture antibody solution applied to the support to result in small, defined spots of immobilized capture antibody.

The capture antibodies are typically directly or indirectly (e.g., via capture tags) restrained on the solid support via covalent or noncovalent interactions (e.g., ionic bonds, hydrophobic interactions, hydrogen bonds, Van der Waals forces, dipole-dipole bonds). In some embodiments, the capture antibodies are covalently attached to the solid support using a homobifunctional or heterobifunctional crosslinker using standard crosslinking methods and conditions. Suitable crosslinkers are commercially available from vendors such as, e.g., Pierce Biotechnology (Rockford, Ill.).

Methods for generating arrays suitable for use in the present invention include, but are not limited to, any technique used to construct protein or nucleic acid arrays. In some embodiments, the capture antibodies are spotted onto an array using a microspotter, which are typically robotic printers equipped with split pins, blunt pins, or ink jet printing. Suitable robotic systems for printing the antibody arrays described herein include the PixSys 5000 robot (Cartesian Technologies; Irvine, Calif.) with ChipMaker2 split pins (TeleChem International; Sunnyvale, Calif.) as well as other robotic printers available from BioRobics (Woburn, Mass.) and Packard Instrument Co. (Meriden, Conn.). Preferably, at least 2, 3, 4, 5, or 6 replicates of each capture antibody dilution are spotted onto the array.

Another method for generating arrays suitable for use in the present invention comprises dispensing a known volume of a capture antibody dilution at each selected array position by contacting a capillary dispenser onto a solid support under conditions effective to draw a defined volume of liquid onto the support, wherein this process is repeated using selected capture antibody dilutions at each selected array position to create a complete array. The method may be practiced in forming a plurality of such arrays, where the solution-depositing step is applied to a selected position on each of a plurality of solid supports at each repeat cycle. A further description of such a method can be found, e.g., in U.S. Pat. No. 5,807,522.

In certain instances, devices for printing on paper can be used to generate the antibody arrays. For example, the desired capture antibody dilution can be loaded into the printhead of a desktop jet printer and printed onto a suitable solid support (see, e.g., Silzel et al., Clin. Chem., 44:2036-2043 (1998)).

In some embodiments, the array generated on the solid support has a density of at least about 5 spots/cm², and preferably at least about 10, 20, 30, 40, 50, 60, 70, 80, 90, 100, 110, 120, 130, 140, 150, 160, 170, 180, 190, 200, 210, 220, 230, 250, 275, 300, 325, 350, 375, 400, 425, 450, 475, 500, 550, 600, 650, 700, 750, 800, 850, 900, 950, 1000, 2000, 3000, 4000, 5000, 6000, 7000, 8000 or 9000, or 10,000 spots/cm².

In certain instances, the spots on the solid support each represents a different capture antibody. In certain other instances, multiple spots on the solid support represent the same capture antibody, e.g., as a dilution series comprising a series of descending capture antibody concentrations.

Additional examples of methods for preparing and constructing antibody arrays on solid supports are described in U.S. Pat. Nos. 6,197,599, 6,777,239, 6,780,582, 6,897,073, 7,179,638, and 7,192,720; U.S. Patent Publication Nos. 20060115810, 20060263837, 20060292680, and 20070054326; and Varnum et al., Methods Mol. Biol., 264:161-172 (2004).

Methods for scanning antibody arrays are known in the art and include, without limitation, any technique used to scan protein or nucleic acid arrays. Microarray scanners suitable for use in the present invention are available from PerkinElmer (Boston, Mass.), Agilent Technologies (Palo Alto, Calif.), Applied Precision (Issaquah, Wash.), GSI Lumonics Inc. (Billerica, Mass.), and Axon Instruments (Union City, Calif.). As a non-limiting example, a GSI ScanArray3000 for fluorescence detection can be used with ImaGene software for quantitation.

### VI. Single Detection Assays

In some embodiments, the assay for detecting the expression and/or activation level of one or more analytes (e.g., one or more signal transduction molecules such as one or more components of the HER3 and/or c-Met signaling pathways) of interest in a cellular extract of cells such as tumor cells is a multiplex, high-throughput two-antibody assay having superior dynamic range. As a non-limiting example, the two antibodies used in the assay can comprise: (1) a capture antibody specific for a particular analyte of interest; and (2) a detection antibody specific for an activated form of the analyte (i.e., activation state-dependent antibody). The activation state-dependent antibody is capable of detecting, for example, the phosphorylation, ubiquitination, and/or complexation state of the analyte. Alternatively, the detection antibody comprises an activation state-independent antibody, which detects the total amount of the analyte in the cellular extract. The activation state-independent antibody is generally capable of detecting both the activated and non-activated forms of the analyte.

In one particular embodiment, the two-antibody assay for detecting the expression or activation level of an analyte of interest comprises:
(i) incubating the cellular extract with one or a plurality of dilution series of capture antibodies to form a plurality of captured analytes;
(ii) incubating the plurality of captured analytes with detection antibodies specific for the corresponding analytes to form a plurality of detectable captured analytes, wherein the detection antibodies comprise activation state-dependent antibodies for detecting the activation (e.g., phosphorylation) level of the analyte or activation state-independent antibodies for detecting the expression level (e.g., total amount) of the analyte;
(iii) incubating the plurality of detectable captured analytes with first and second members of a signal amplification pair to generate an amplified signal; and
(iv) detecting the amplified signal generated from the first and second members of the signal amplification pair.

The two-antibody assays described herein are typically antibody-based arrays which comprise a plurality of different capture antibodies at a range of capture antibody concentrations that are coupled to the surface of a solid support in different addressable locations. Examples of suitable solid supports for use in the present invention are described above.

The capture antibodies and detection antibodies are preferably selected to minimize competition between them with respect to analyte binding (i.e., both capture and detection antibodies can simultaneously bind their corresponding signal transduction molecules).

In one embodiment, the detection antibodies comprise a first member of a binding pair (e.g., biotin) and the first member of the signal amplification pair comprises a second member of the binding pair (e.g., streptavidin). The binding pair members can be coupled directly or indirectly to the detection antibodies or to the first member of the signal amplification pair using methods well-known in the art. In certain instances, the first member of the signal amplification pair is a peroxidase (e.g., horseradish peroxidase (HRP), catalase, chloroperoxidase, cytochrome c peroxidase, eosinophil peroxidase, glutathione peroxidase, lactoperoxidase, myeloperoxidase, thyroid peroxidase, deiodinase, etc.), and the second member of the signal amplification pair is a tyramide reagent (e.g., biotin-tyramide). In these instances, the amplified signal is generated by peroxidase oxidization of the tyramide reagent to produce an activated tyramide in the presence of hydrogen peroxide (H₂O₂).

The activated tyramide is either directly detected or detected upon the addition of a signal-detecting reagent such as, for example, a streptavidin-labeled fluorophore or a combination of a streptavidin-labeled peroxidase and a chromogenic reagent. Examples of fluorophores suitable for use in the present invention include, but are not limited to, an Alexa Fluor® dye (e.g., Alexa Fluor® 555), fluorescein, fluorescein isothiocyanate (FITC), Oregon Green™; rhodamine, Texas red, tetrarhodamine isothiocynate (TRITC), a CyDye™ fluor (e.g., Cy2, Cy3, Cy5), and the like. The streptavidin label can be coupled directly or indirectly to the fluorophore or peroxidase using methods well-known in the art. Non-limiting examples of chromogenic reagents suitable for use in the present invention include 3,3′,5,5′-tetramethylbenzidine (TMB), 3,3′-diaminobenzidine (DAB), 2,2′-azino-bis(3-ethylbenzothiazoline-6-sulfonic acid) (ABTS),4-chloro-1-napthol (4CN), and/or porphyrinogen.

An exemplary protocol for performing the two-antibody assays described herein is provided in Example 3 of PCT Publication No. WO2009/108637, the disclosure of which is herein incorporated by reference in its entirety for all purposes.

In another embodiment of a two-antibody approach, the present invention provides a method for detecting the expression or activation level of a truncated receptor, the method comprising:
(i) incubating the cellular extract with a plurality of beads specific for an extracellular domain (ECD) binding region of a full-length receptor;
(ii) removing the plurality of beads from the cellular extract, thereby removing the full-length receptor to form a cellular extract devoid of the full-length receptor;
(iii) incubating the cellular extract devoid of the full-length receptor with a dilution series of one or a plurality of capture antibodies specific for an intracellular domain (ICD) binding region of the full-length receptor to form a plurality of captured truncated receptors;
(iv) incubating the plurality of captured truncated receptors with detection antibodies specific for an ICD binding region of the full-length receptor to form a plurality of detectable captured truncated receptors, wherein the detection antibodies comprise activation state-dependent antibodies for detecting the activation (e.g., phosphorylation) level of the truncated receptor or activation state-independent antibodies for detecting the expression level (e.g., total amount) of the truncated receptor;
(v) incubating the plurality of detectable captured truncated receptors with first and second members of a signal amplification pair to generate an amplified signal; and
(vi) detecting an amplified signal generated from the first and second members of the signal amplification pair.

In certain embodiments, the truncated receptor is p95HER2 and the full-length receptor is HER2. In other embodiments, the truncated receptor is a truncated form of HER3 and the full-length receptor is HER3. In yet other embodiments, the truncated receptor is a truncated form of c-Met and the full-length receptor is c-Met. In further embodiments, the plurality of beads specific for an extracellular domain (ECD) binding region comprises a streptavidin-biotin pair, wherein the biotin is attached to the bead and the biotin is attached to an antibody (e.g., wherein the antibody is specific for the ECD binding region of the full-length receptor).

FIG. 14A of PCT Publication No. WO2009/108637, the disclosure of which is herein incorporated by reference in its entirety for all purposes, shows that beads coated with an antibody directed to the extracellular domain (ECD) of a receptor of interest binds the full-length receptor (e.g., HER2), but not the truncated receptor (e.g., p95HER2) to remove any full-length receptor from the assay. FIG. 14B of PCT Publication No. WO2009/108637 shows that the truncated receptor (e.g., p95HER2), once bound to a capture antibody, may then be detected by a detection antibody that is specific for the intracellular domain (ICD) of the full-length receptor (e.g., HER2). The detection antibody may be directly conjugated to horseradish peroxidase (HRP). Tyramide signal amplification (TSA) may then be performed to generate a signal to be detected. The expression level or activation state of the truncated receptor (e.g., p95HER2) can be interrogated to determine, e.g., its total concentration or its phosphorylation state, ubiquitination state, and/or complexation state.

In another embodiment, the present invention provides kits for performing the two-antibody assays described above comprising: (a) a dilution series of one or a plurality of capture antibodies restrained on a solid support; and (b) one or a plurality of detection antibodies (e.g., activation state-independent antibodies and/or activation state-dependent antibodies). In some instances, the kits can further contain instructions for methods of using the kit to detect the expression levels and/or activation states of one or a plurality of signal transduction molecules of cells such as tumor cells. The kits may also contain any of the additional reagents described above with respect to performing the specific methods of the present invention such as, for example, first and second members of the signal amplification pair, tyramide signal amplification reagents, wash buffers, etc.

### VII. Proximity Dual Detection Assays

In some embodiments, the assay for detecting the expression and/or activation level of one or more analytes (e.g., one or more signal transduction molecules such as one or more components of the HER3 and/or c-Met signaling pathways) of interest in a cellular extract of cells such as tumor cells is a multiplex, high-throughput proximity (i.e., three-antibody) assay having superior dynamic range. As a non-limiting example, the three antibodies used in the proximity assay can comprise: (1) a capture antibody specific for a particular analyte of interest; (2) a detection antibody specific for an activated form of the analyte (i.e., activation state-dependent antibody); and (3) a detection antibody which detects the total amount of the analyte (i.e., activation state-independent antibody). The activation state-dependent antibody is capable of detecting, e.g., the phosphorylation, ubiquitination, and/or complexation state of the analyte, while the activation state-independent antibody is capable of detecting the total amount (i.e., both the activated and non-activated forms) of the analyte. The proximity assay described herein is also known as a Collaborative Enzyme Enhanced Reactive ImmunoAssay (CEER) or a Collaborative Proximity Immunoassay (COPIA).

In one particular embodiment, the proximity assay for detecting the activation level or status of an analyte of interest comprises:
(i) incubating the cellular extract with one or a plurality of dilution series of capture antibodies to form a plurality of captured analytes;
(ii) incubating the plurality of captured analytes with detection antibodies comprising one or a plurality of activation state-independent antibodies and one or a plurality of activation state-dependent antibodies specific for the corresponding analytes to form a plurality of detectable captured analytes,
wherein the activation state-independent antibodies are labeled with a facilitating moiety, the activation state-dependent antibodies are labeled with a first member of a signal amplification pair, and the facilitating moiety generates an oxidizing agent which channels to and reacts with the first member of the signal amplification pair;
(iii) incubating the plurality of detectable captured analytes with a second member of the signal amplification pair to generate an amplified signal; and
(iv) detecting the amplified signal generated from the first and second members of the signal amplification pair.

In another particular embodiment, the proximity assay for detecting the activation level or status of an analyte of interest that is a truncated receptor comprises:
(i) incubating the cellular extract with a plurality of beads specific for an extracellular domain (ECD) binding region of a full-length receptor;
(ii) removing the plurality of beads from the cellular extract, thereby removing the full-length receptor to form a cellular extract devoid of the full-length receptor;
(iii) incubating the cellular extract devoid of the full-length receptor with one or a plurality of capture antibodies specific for an intracellular domain (ICD) binding region of the full-length receptor to form a plurality of captured truncated receptors;
(iv) incubating the plurality of captured truncated receptors with detection antibodies comprising one or a plurality of activation state-independent antibodies and one or a plurality of activation state-dependent antibodies specific for an ICD binding region of the full-length receptor to form a plurality of detectable captured truncated receptors,
wherein the activation state-independent antibodies are labeled with a facilitating moiety, the activation state-dependent antibodies are labeled with a first member of a signal amplification pair, and the facilitating moiety generates an oxidizing agent which channels to and reacts with the first member of the signal amplification pair;
(v) incubating the plurality of detectable captured truncated receptors with a second member of the signal amplification pair to generate an amplified signal; and
(vi) detecting the amplified signal generated from the first and second members of the signal amplification pair.

In certain embodiments, the truncated receptor is p95HER2 and the full-length receptor is HER2. In other embodiments, the truncated receptor is a truncated form of HER3 and the full-length receptor is HER3. In yet other embodiments, the truncated receptor is a truncated form of c-Met and the full-length receptor is c-Met. In further embodiments, the plurality of beads specific for an extracellular domain (ECD) binding region comprises a streptavidin-biotin pair, wherein the biotin is attached to the bead and the biotin is attached to an antibody (e.g., wherein the antibody is specific for the ECD binding region of the full-length receptor).

In alternative embodiments, the activation state-dependent antibodies can be labeled with a facilitating moiety and the activation state-independent antibodies can be labeled with a first member of a signal amplification pair.

As another non-limiting example, the three antibodies used in the proximity assay can comprise: (1) a capture antibody specific for a particular analyte of interest; (2) a first detection antibody which detects the total amount of the analyte (i.e., a first activation state-independent antibody); and (3) a second detection antibody which detects the total amount of the analyte (i.e., a second activation state-independent antibody). In preferred embodiments, the first and second activation state-independent antibodies recognize different (e.g., distinct) epitopes on the analyte.

In one particular embodiment, the proximity assay for detecting the expression level of an analyte of interest comprises:
(i) incubating the cellular extract with one or a plurality of dilution series of capture antibodies to form a plurality of captured analytes;
(ii) incubating the plurality of captured analytes with detection antibodies comprising one or a plurality of first and second activation state-independent antibodies specific for the corresponding analytes to form a plurality of detectable captured analytes,
wherein the first activation state-independent antibodies are labeled with a facilitating moiety, the second activation state-independent antibodies are labeled with a first member of a signal amplification pair, and the facilitating moiety generates an oxidizing agent which channels to and reacts with the first member of the signal amplification pair;
(iii) incubating the plurality of detectable captured analytes with a second member of the signal amplification pair to generate an amplified signal; and
(iv) detecting the amplified signal generated from the first and second members of the signal amplification pair.

In another particular embodiment, the proximity assay for detecting the expression level of an analyte of interest that is a truncated receptor comprises:
(i) incubating the cellular extract with a plurality of beads specific for an extracellular domain (ECD) binding region of a full-length receptor;
(ii) removing the plurality of beads from the cellular extract, thereby removing the full-length receptor to form a cellular extract devoid of the full-length receptor;
(iii) incubating the cellular extract devoid of the full-length receptor with one or a plurality of capture antibodies specific for an intracellular domain (ICD) binding region of the full-length receptor to form a plurality of captured truncated receptors;
(iv) incubating the plurality of captured truncated receptors with detection antibodies comprising one or a plurality of first and second activation state-independent antibodies specific for an ICD binding region of the full-length receptor to form a plurality of detectable captured truncated receptors,
wherein the first activation state-independent antibodies are labeled with a facilitating moiety, the second activation state-independent antibodies are labeled with a first member of a signal amplification pair, and the facilitating moiety generates an oxidizing agent which channels to and reacts with the first member of the signal amplification pair;
(v) incubating the plurality of detectable captured truncated receptors with a second member of the signal amplification pair to generate an amplified signal; and
(vi) detecting the amplified signal generated from the first and second members of the signal amplification pair.

In certain embodiments, the truncated receptor is p95HER2 and the full-length receptor is HER2. In other embodiments, the truncated receptor is a truncated form of HER3 and the full-length receptor is HER3. In yet other embodiments, the truncated receptor is a truncated form of c-Met and the full-length receptor is c-Met. In further embodiments, the plurality of beads specific for an extracellular domain (ECD) binding region comprises a streptavidin-biotin pair, wherein the biotin is attached to the bead and the biotin is attached to an antibody (e.g., wherein the antibody is specific for the ECD binding region of the full-length receptor).

In alternative embodiments, the first activation state-independent antibodies can be labeled with a first member of a signal amplification pair and the second activation state-independent antibodies can be labeled with a facilitating moiety.

The proximity assays described herein are typically antibody-based arrays which comprise one or a plurality of different capture antibodies at a range of capture antibody concentrations that are coupled to the surface of a solid support in different addressable locations. Examples of suitable solid supports for use in the present invention are described above.

The capture antibodies, activation state-independent antibodies, and activation state-dependent antibodies are preferably selected to minimize competition between them with respect to analyte binding (i.e., all antibodies can simultaneously bind their corresponding signal transduction molecules).

In some embodiments, activation state-independent antibodies for detecting activation levels of one or more of the analytes or, alternatively, first activation state-independent antibodies for detecting expression levels of one or more of the analytes further comprise a detectable moiety. In such instances, the amount of the detectable moiety is correlative to the amount of one or more of the analytes in the cellular extract. Examples of detectable moieties include, but are not limited to, fluorescent labels, chemically reactive labels, enzyme labels, radioactive labels, and the like. Preferably, the detectable moiety is a fluorophore such as an Alexa Fluor® dye (e.g., Alexa Fluor® 647), fluorescein, fluorescein isothiocyanate (FITC), Oregon Green™; rhodamine, Texas red, tetrarhodamine isothiocynate (TRITC), a CyDye™ fluor (e.g., Cy2, Cy3, Cy5), and the like. The detectable moiety can be coupled directly or indirectly to the activation state-independent antibodies using methods well-known in the art.

In certain instances, activation state-independent antibodies for detecting activation levels of one or more of the analytes or, alternatively, first activation state-independent antibodies for detecting expression levels of one or more of the analytes are directly labeled with the facilitating moiety. The facilitating moiety can be coupled to activation state-independent antibodies using methods well-known in the art. A suitable facilitating moiety for use in the present invention includes any molecule capable of generating an oxidizing agent which channels to (i.e., is directed to) and reacts with (i.e., binds, is bound by, or forms a complex with) another molecule in proximity (i.e., spatially near or close) to the facilitating moiety. Examples of facilitating moieties include, without limitation, enzymes such as glucose oxidase or any other enzyme that catalyzes an oxidation/reduction reaction involving molecular oxygen (O₂) as the electron acceptor, and photosensitizers such as methylene blue, rose bengal, porphyrins, squarate dyes, phthalocyanines, and the like. Non-limiting examples of oxidizing agents include hydrogen peroxide (H₂O₂), a singlet oxygen, and any other compound that transfers oxygen atoms or gains electrons in an oxidation/reduction reaction. Preferably, in the presence of a suitable substrate (e.g., glucose, light, etc.), the facilitating moiety (e.g., glucose oxidase, photosensitizer, etc.) generates an oxidizing agent (e.g., hydrogen peroxide (H₂O₂), single oxygen, etc.) which channels to and reacts with the first member of the signal amplification pair (e.g., horseradish peroxidase (HRP), hapten protected by a protecting group, an enzyme inactivated by thioether linkage to an enzyme inhibitor, etc.) when the two moieties are in proximity to each other.

In certain other instances, activation state-independent antibodies for detecting activation levels of one or more of the analytes or, alternatively, first activation state-independent antibodies for detecting expression levels of one or more of the analytes are indirectly labeled with the facilitating moiety via hybridization between an oligonucleotide linker conjugated to the activation state-independent antibodies and a complementary oligonucleotide linker conjugated to the facilitating moiety. The oligonucleotide linkers can be coupled to the facilitating moiety or to the activation state-independent antibodies using methods well-known in the art. In some embodiments, the oligonucleotide linker conjugated to the facilitating moiety has 100% complementarity to the oligonucleotide linker conjugated to the activation state-independent antibodies. In other embodiments, the oligonucleotide linker pair comprises at least one, two, three, four, five, six, or more mismatch regions, e.g., upon hybridization under stringent hybridization conditions. One skilled in the art will appreciate that activation state-independent antibodies specific for different analytes can either be conjugated to the same oligonucleotide linker or to different oligonucleotide linkers.

The length of the oligonucleotide linkers that are conjugated to the facilitating moiety or to the activation state-independent antibodies can vary. In general, the linker sequence can be at least about 5, 10, 15, 20, 25, 30, 35, 40, 45, 50, 75, or 100 nucleotides in length. Typically, random nucleic acid sequences are generated for coupling. As a non-limiting example, a library of oligonucleotide linkers can be designed to have three distinct contiguous domains: a spacer domain; signature domain; and conjugation domain. Preferably, the oligonucleotide linkers are designed for efficient coupling without destroying the function of the facilitating moiety or activation state-independent antibodies to which they are conjugated.

The oligonucleotide linker sequences can be designed to prevent or minimize any secondary structure formation under a variety of assay conditions. Melting temperatures are typically carefully monitored for each segment within the linker to allow their participation in the overall assay procedures. Generally, the range of melting temperatures of the segment of the linker sequence is between 1-10° C. Computer algorithms (e.g., OLIGO 6.0) for determining the melting temperature, secondary structure, and hairpin structure under defined ionic concentrations can be used to analyze each of the three different domains within each linker. The overall combined sequences can also be analyzed for their structural characterization and their comparability to other conjugated oligonucleotide linker sequences, e.g., whether they will hybridize under stringent hybridization conditions to a complementary oligonucleotide linker.

The spacer region of the oligonucleotide linker provides adequate separation of the conjugation domain from the oligonucleotide crosslinking site. The conjugation domain functions to link molecules labeled with a complementary oligonucleotide linker sequence to the conjugation domain via nucleic acid hybridization. The nucleic acid-mediated hybridization can be performed either before or after antibody-analyte (i.e., antigen) complex formation, providing a more flexible assay format. Unlike many direct antibody conjugation methods, linking relatively small oligonucleotides to antibodies or other molecules has minimal impact on the specific affinity of antibodies towards their target analyte or on the function of the conjugated molecules.

In some embodiments, the signature sequence domain of the oligonucleotide linker can be used in complex multiplexed protein assays. Multiple antibodies can be conjugated with oligonucleotide linkers with different signature sequences. In multiplex immunoassays, reporter oligonucleotide sequences labeled with appropriate probes can be used to detect cross-reactivity between antibodies and their antigens in the multiplex assay format.

Oligonucleotide linkers can be conjugated to antibodies or other molecules using several different methods. For example, oligonucleotide linkers can be synthesized with a thiol group on either the 5′ or 3′ end. The thiol group can be deprotected using reducing agents (e.g., TCEP-HCl) and the resulting linkers can be purified by using a desalting spin column. The resulting deprotected oligonucleotide linkers can be conjugated to the primary amines of antibodies or other types of proteins using heterobifunctional cross linkers such as SMCC. Alternatively, 5′-phosphate groups on oligonucleotides can be treated with water-soluble carbodiimide EDC to form phosphate esters and subsequently coupled to amine-containing molecules. In certain instances, the diol on the 3′-ribose residue can be oxidized to aldehyde groups and then conjugated to the amine groups of antibodies or other types of proteins using reductive amination. In certain other instances, the oligonucleotide linker can be synthesized with a biotin modification on either the 3′ or 5′ end and conjugated to streptavidin-labeled molecules.

Oligonucleotide linkers can be synthesized using any of a variety of techniques known in the art, such as those described in Usman et al., J. Am. Chem. Soc., 109:7845 (1987); Scaringe et al., Nucl. Acids Res., 18:5433 (1990); Wincott et al., Nucl. Acids Res., 23:2677-2684 (1995); and Wincott et al., Methods Mol. Bio., 74:59 (1997). In general, the synthesis of oligonucleotides makes use of common nucleic acid protecting and coupling groups, such as dimethoxytrityl at the 5′-end and phosphoramidites at the 3′-end. Suitable reagents for oligonucleotide synthesis, methods for nucleic acid deprotection, and methods for nucleic acid purification are known to those of skill in the art.

In certain instances, activation state-dependent antibodies for detecting activation levels of one or more of the analytes or, alternatively, second activation state-independent antibodies for detecting expression levels of one or more of the analytes are directly labeled with the first member of the signal amplification pair. The signal amplification pair member can be coupled to activation state-dependent antibodies to detect activation levels or second activation state-independent antibodies to detect expression levels using methods well-known in the art. In certain other instances, activation state-dependent antibodies or second activation state-independent antibodies are indirectly labeled with the first member of the signal amplification pair via binding between a first member of a binding pair conjugated to the activation state-dependent antibodies or second activation state-independent antibodies and a second member of the binding pair conjugated to the first member of the signal amplification pair. The binding pair members (e.g., biotin/streptavidin) can be coupled to the signal amplification pair member or to the activation state-dependent antibodies or second activation state-independent antibodies using methods well-known in the art. Examples of signal amplification pair members include, but are not limited to, peroxidases such horseradish peroxidase (HRP), catalase, chloroperoxidase, cytochrome c peroxidase, eosinophil peroxidase, glutathione peroxidase, lactoperoxidase, myeloperoxidase, thyroid peroxidase, deiodinase, and the like. Other examples of signal amplification pair members include haptens protected by a protecting group and enzymes inactivated by thioether linkage to an enzyme inhibitor.

In one example of proximity channeling, the facilitating moiety is glucose oxidase (GO) and the first member of the signal amplification pair is horseradish peroxidase (HRP). When the GO is contacted with a substrate such as glucose, it generates an oxidizing agent (i.e., hydrogen peroxide (H₂O₂)). If the HRP is within channeling proximity to the GO, the H₂O₂ generated by the GO is channeled to and complexes with the HRP to form an HRP-H₂O₂ complex, which, in the presence of the second member of the signal amplification pair (e.g., a chemiluminescent substrate such as luminol or isoluminol or a fluorogenic substrate such as tyramide (e.g., biotin-tyramide), homovanillic acid, or 4-hydroxyphenyl acetic acid), generates an amplified signal. Methods of using GO and HRP in a proximity assay are described in, e.g., Langry et al., U.S. Dept. of Energy Report No. UCRL-ID-136797 (1999). When biotin-tyramide is used as the second member of the signal amplification pair, the HRP-H₂O₂ complex oxidizes the tyramide to generate a reactive tyramide radical that covalently binds nearby nucleophilic residues. The activated tyramide is either directly detected or detected upon the addition of a signal-detecting reagent such as, for example, a streptavidin-labeled fluorophore or a combination of a streptavidin-labeled peroxidase and a chromogenic reagent. Examples of fluorophores suitable for use in the present invention include, but are not limited to, an Alexa Fluor® dye (e.g., Alexa Fluor® 555), fluorescein, fluorescein isothiocyanate (FITC), Oregon Green™; rhodamine, Texas red, tetrarhodamine isothiocynate (TRITC), a CyDye™ fluor (e.g., Cy2, Cy3, Cy5), and the like. The streptavidin label can be coupled directly or indirectly to the fluorophore or peroxidase using methods well-known in the art. Non-limiting examples of chromogenic reagents suitable for use in the present invention include 3,3′,5,5′-tetramethylbenzidine (TMB), 3,3′-diaminobenzidine (DAB), 2,2′-azino-bis(3-ethylbenzothiazoline-6-sulfonic acid) (ABTS), 4-chloro-1-napthol (4CN), and/or porphyrinogen.

In some embodiments, the glucose oxidase (GO) and the detection antibodies (e.g., activation state-independent antibodies) can be conjugated to a sulfhydryl-activated dextran molecule as described in, e.g., Examples 16-17 of PCT Publication No. WO 2009/108637, the disclosure of which is herein incorporated by reference in its entirety for all purposes. The sulfhydryl-activated dextran molecule typically has a molecular weight of about 500 kDa (e.g., about 250, 300, 350, 400, 450, 500, 550, 600, 650, 700, or 750 kDa). In certain other embodiments, the horseradish peroxidase (HRP) and the detection antibodies (e.g., activation state-dependent antibodies) can be conjugated to a sulfhydryl-activated dextran molecule. The sulfhydryl-activated dextran molecule typically has a molecular weight of about 70 kDa (e.g., about 40, 45, 50, 55, 60, 65, 70, 75, 80, 85, 90, 95, or 100 kDa).

In another example of proximity channeling, the facilitating moiety is a photosensitizer and the first member of the signal amplification pair is a large molecule labeled with multiple haptens that are protected with protecting groups that prevent binding of the haptens to a specific binding partner (e.g., ligand, antibody, etc.). For example, the signal amplification pair member can be a dextran molecule labeled with protected biotin, coumarin, and/or fluorescein molecules. Suitable protecting groups include, but are not limited to, phenoxy-, analino-, olefin-, thioether-, and selenoether-protecting groups. Additional photosensitizers and protected hapten molecules suitable for use in the proximity assays of the present invention are described in U.S. Pat. No. 5,807,675. When the photosensitizer is excited with light, it generates an oxidizing agent (i.e., singlet oxygen). If the hapten molecules are within channeling proximity to the photosensitizer, the singlet oxygen generated by the photosensitizer is channeled to and reacts with thioethers on the protecting groups of the haptens to yield carbonyl groups (ketones or aldehydes) and sulphinic acid, releasing the protecting groups from the haptens. The unprotected haptens are then available to specifically bind to the second member of the signal amplification pair (e.g., a specific binding partner that can generate a detectable signal). For example, when the hapten is biotin, the specific binding partner can be an enzyme-labeled streptavidin. Exemplary enzymes include alkaline phosphatase, β-galactosidase, HRP, etc. After washing to remove unbound reagents, the detectable signal can be generated by adding a detectable (e.g., fluorescent, chemiluminescent, chromogenic, etc.) substrate of the enzyme and detected using suitable methods and instrumentation known in the art. Alternatively, the detectable signal can be amplified using tyramide signal amplification and the activated tyramide either directly detected or detected upon the addition of a signal-detecting reagent as described above.

In yet another example of proximity channeling, the facilitating moiety is a photosensitizer and the first member of the signal amplification pair is an enzyme-inhibitor complex. The enzyme and inhibitor (e.g., phosphonic acid-labeled dextran) are linked together by a cleavable linker (e.g., thioether). When the photosensitizer is excited with light, it generates an oxidizing agent (i.e., singlet oxygen). If the enzyme-inhibitor complex is within channeling proximity to the photosensitizer, the singlet oxygen generated by the photosensitizer is channeled to and reacts with the cleavable linker, releasing the inhibitor from the enzyme, thereby activating the enzyme. An enzyme substrate is added to generate a detectable signal, or alternatively, an amplification reagent is added to generate an amplified signal.

In a further example of proximity channeling, the facilitating moiety is HRP, the first member of the signal amplification pair is a protected hapten or an enzyme-inhibitor complex as described above, and the protecting groups comprise p-alkoxy phenol. The addition of phenylenediamine and H₂O₂ generates a reactive phenylene diimine which channels to the protected hapten or the enzyme-inhibitor complex and reacts with p-alkoxy phenol protecting groups to yield exposed haptens or a reactive enzyme. The amplified signal is generated and detected as described above (see, e.g., U.S. Pat. Nos. 5,532,138 and 5,445,944).

An exemplary protocol for performing the proximity assays described herein is provided in Example 4 of PCT Publication No. WO2009/108637, the disclosure of which is herein incorporated by reference in its entirety for all purposes.

In another embodiment, the present invention provides kits for performing the proximity assays described above comprising: (a) a dilution series of one or a plurality of capture antibodies restrained on a solid support; and (b) one or a plurality of detection antibodies (e.g., a combination of activation state-independent antibodies and activation state-dependent antibodies for detecting activation levels and/or a combination of first and second activation state-independent antibodies for detecting expression levels). In some instances, the kits can further contain instructions for methods of using the kit to detect the expression and/or activation status of one or a plurality of signal transduction molecules of cells such as tumor cells. The kits may also contain any of the additional reagents described above with respect to performing the specific methods of the present invention such as, for example, first and second members of the signal amplification pair, tyramide signal amplification reagents, substrates for the facilitating moiety, wash buffers, etc.

### VIII. Production of Antibodies

The generation and selection of antibodies not already commercially available for analyzing the expression and/or activation levels of signal transduction molecules (e.g., HER3 and/or c-MET signaling pathway components) in cells such as non-small cell lung cancer tumor cells in accordance with the present invention can be accomplished several ways. For example, one way is to express and/or purify a polypeptide of interest (i.e., antigen) using protein expression and purification methods known in the art, while another way is to synthesize the polypeptide of interest using solid phase peptide synthesis methods known in the art. See, e.g., Guide to Protein Purification, Murray P. Deutcher, ed., Meth. Enzymol., Vol. 182 (1990); Solid Phase Peptide Synthesis, Greg B. Fields, ed., Meth. Enzymol., Vol. 289 (1997); Kiso et al., Chem. Pharm. Bull., 38:1192-99 (1990); Mostafavi et al., Biomed. Pept. Proteins Nucleic Acids, 1:255-60, (1995); and Fujiwara et al., Chem. Pharm. Bull., 44:1326-31 (1996). The purified or synthesized polypeptide can then be injected, for example, into mice or rabbits, to generate polyclonal or monoclonal antibodies. One skilled in the art will recognize that many procedures are available for the production of antibodies, for example, as described in Antibodies, A Laboratory Manual, Harlow and Lane, Eds., Cold Spring Harbor Laboratory, Cold Spring Harbor, N.Y. (1988). One skilled in the art will also appreciate that binding fragments or Fab fragments which mimic (e.g., retain the functional binding regions of) antibodies can also be prepared from genetic information by various procedures. See, e.g., Antibody Engineering: A Practical Approach, Borrebaeck, Ed., Oxford University Press, Oxford (1995); and Huse et al., J. Immunol., 149:3914-3920 (1992).

In addition, numerous publications have reported the use of phage display technology to produce and screen libraries of polypeptides for binding to a selected target antigen (see, e.g, Cwirla et al., Proc. Natl. Acad. Sci. USA, 87:6378-6382 (1990); Devlin et al., Science, 249:404-406 (1990); Scott et al., Science, 249:386-388 (1990); and Ladner et al., U.S. Pat. No. 5,571,698). A basic concept of phage display methods is the establishment of a physical association between a polypeptide encoded by the phage DNA and a target antigen. This physical association is provided by the phage particle, which displays a polypeptide as part of a capsid enclosing the phage genome which encodes the polypeptide. The establishment of a physical association between polypeptides and their genetic material allows simultaneous mass screening of very large numbers of phage bearing different polypeptides. Phage displaying a polypeptide with affinity to a target antigen bind to the target antigen and these phage are enriched by affinity screening to the target antigen. The identity of polypeptides displayed from these phage can be determined from their respective genomes. Using these methods, a polypeptide identified as having a binding affinity for a desired target antigen can then be synthesized in bulk by conventional means (see, e.g., U.S. Pat. No. 6,057,098).

The antibodies that are generated by these methods can then be selected by first screening for affinity and specificity with the purified polypeptide antigen of interest and, if required, comparing the results to the affinity and specificity of the antibodies with other polypeptide antigens that are desired to be excluded from binding. The screening procedure can involve immobilization of the purified polypeptide antigens in separate wells of microtiter plates. The solution containing a potential antibody or group of antibodies is then placed into the respective microtiter wells and incubated for about 30 minutes to 2 hours. The microtiter wells are then washed and a labeled secondary antibody (e.g., an anti-mouse antibody conjugated to alkaline phosphatase if the raised antibodies are mouse antibodies) is added to the wells and incubated for about 30 minutes and then washed. Substrate is added to the wells and a color reaction will appear where antibody to the immobilized polypeptide antigen is present.

The antibodies so identified can then be further analyzed for affinity and specificity. In the development of immunoassays for a target protein, the purified target protein acts as a standard with which to judge the sensitivity and specificity of the immunoassay using the antibodies that have been selected. Because the binding affinity of various antibodies may differ, e.g., certain antibody combinations may interfere with one another sterically, assay performance of an antibody may be a more important measure than absolute affinity and specificity of that antibody.

Those skilled in the art will recognize that many approaches can be taken in producing antibodies or binding fragments and screening and selecting for affinity and specificity for the various polypeptides of interest, but these approaches do not change the scope of the present invention.

A. Polyclonal Antibodies

Polyclonal antibodies are preferably raised in animals by multiple subcutaneous (sc) or intraperitoneal (ip) injections of a polypeptide of interest and an adjuvant. It may be useful to conjugate the polypeptide of interest to a protein carrier that is immunogenic in the species to be immunized, such as, e.g., keyhole limpet hemocyanin, serum albumin, bovine thyroglobulin, or soybean trypsin inhibitor using a bifunctional or derivatizing agent. Non-limiting examples of bifunctional or derivatizing agents include maleimidobenzoyl sulfosuccinimide ester (conjugation through cysteine residues), N-hydroxysuccinimide (conjugation through lysine residues), glutaraldehyde, succinic anhydride, SOCl₂, and R₁N═C═NR, wherein R and R₁ are different alkyl groups.

Animals are immunized against the polypeptide of interest or an immunogenic conjugate or derivative thereof by combining, e.g., 100 μg (for rabbits) or 5 μg (for mice) of the antigen or conjugate with 3 volumes of Freund's complete adjuvant and injecting the solution intradermally at multiple sites. One month later, the animals are boosted with about 1/5 to 1/10 the original amount of polypeptide or conjugate in Freund's incomplete adjuvant by subcutaneous injection at multiple sites. Seven to fourteen days later, the animals are bled and the serum is assayed for antibody titer. Animals are typically boosted until the titer plateaus. Preferably, the animal is boosted with the conjugate of the same polypeptide, but conjugation to a different immunogenic protein and/or through a different cross-linking reagent may be used. Conjugates can also be made in recombinant cell culture as fusion proteins. In certain instances, aggregating agents such as alum can be used to enhance the immune response.

B. Monoclonal Antibodies

Monoclonal antibodies are generally obtained from a population of substantially homogeneous antibodies, i.e., the individual antibodies comprising the population are identical except for possible naturally-occurring mutations that may be present in minor amounts. Thus, the modifier “monoclonal” indicates the character of the antibody as not being a mixture of discrete antibodies. For example, monoclonal antibodies can be made using the hybridoma method described by Kohler et al., Nature, 256:495 (1975) or by any recombinant DNA method known in the art (see, e.g., U.S. Pat. No. 4,816,567).

In the hybridoma method, a mouse or other appropriate host animal (e.g., hamster) is immunized as described above to elicit lymphocytes that produce or are capable of producing antibodies which specifically bind to the polypeptide of interest used for immunization. Alternatively, lymphocytes are immunized in vitro. The immunized lymphocytes are then fused with myeloma cells using a suitable fusing agent, such as polyethylene glycol, to form hybridoma cells (see, e.g., Goding, Monoclonal Antibodies: Principles and Practice, Academic Press, pp. 59-103 (1986)). The hybridoma cells thus prepared are seeded and grown in a suitable culture medium that preferably contains one or more substances which inhibit the growth or survival of the unfused, parental myeloma cells. For example, if the parental myeloma cells lack the enzyme hypoxanthine guanine phosphoribosyl transferase (HGPRT), the culture medium for the hybridoma cells will typically include hypoxanthine, aminopterin, and thymidine (HAT medium), which prevent the growth of HGPRT-deficient cells.

Preferred myeloma cells are those that fuse efficiently, support stable high-level production of antibody by the selected antibody-producing cells, and/or are sensitive to a medium such as HAT medium. Examples of such preferred myeloma cell lines for the production of human monoclonal antibodies include, but are not limited to, murine myeloma lines such as those derived from MOPC-21 and MPC-11 mouse tumors (available from the Salk Institute Cell Distribution Center; San Diego, Calif.), SP-2 or X63-Ag8-653 cells (available from the American Type Culture Collection; Rockville, Md.), and human myeloma or mouse-human heteromyeloma cell lines (see, e.g., Kozbor, J. Immunol., 133:3001 (1984); and Brodeur et al., Monoclonal Antibody Production Techniques and Applications, Marcel Dekker, Inc., New York, pp. 51-63 (1987)).

The culture medium in which hybridoma cells are growing can be assayed for the production of monoclonal antibodies directed against the polypeptide of interest. Preferably, the binding specificity of monoclonal antibodies produced by hybridoma cells is determined by immunoprecipitation or by an in vitro binding assay, such as a radioimmunoassay (RIA) or an enzyme-linked immunoabsorbent assay (ELISA). The binding affinity of monoclonal antibodies can be determined using, e.g., the Scatchard analysis of Munson et al., Anal. Biochem., 107:220 (1980).

After hybridoma cells are identified that produce antibodies of the desired specificity, affinity, and/or activity, the clones may be subcloned by limiting dilution procedures and grown by standard methods (see, e.g., Goding, Monoclonal Antibodies: Principles and Practice, Academic Press, pp. 59-103 (1986)). Suitable culture media for this purpose include, for example, D-MEM or RPMI-1640 medium. In addition, the hybridoma cells may be grown in vivo as ascites tumors in an animal. The monoclonal antibodies secreted by the subclones can be separated from the culture medium, ascites fluid, or serum by conventional antibody purification procedures such as, for example, protein A-Sepharose, hydroxylapatite chromatography, gel electrophoresis, dialysis, or affinity chromatography.

DNA encoding the monoclonal antibodies can be readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as E. coli cells, simian COS cells, Chinese Hamster Ovary (CHO) cells, or myeloma cells that do not otherwise produce antibody, to induce the synthesis of monoclonal antibodies in the recombinant host cells. See, e.g., Skerra et al., Curr. Opin. Immunol., 5:256-262 (1993); and Pluckthun, Immunol Rev., 130:151-188 (1992). The DNA can also be modified, for example, by substituting the coding sequence for human heavy chain and light chain constant domains in place of the homologous murine sequences (see, e.g., U.S. Pat. No. 4,816,567; and Morrison et al., Proc. Natl. Acad. Sci. USA, 81:6851 (1984)), or by covalently joining to the immunoglobulin coding sequence all or part of the coding sequence for a non-immunoglobulin polypeptide.

In a further embodiment, monoclonal antibodies or antibody fragments can be isolated from antibody phage libraries generated using the techniques described in, for example, McCafferty et al., Nature, 348:552-554 (1990); Clackson et al., Nature, 352:624-628 (1991); and Marks et al., J. Mol. Biol., 222:581-597 (1991). The production of high affinity (nM range) human monoclonal antibodies by chain shuffling is described in Marks et al., BioTechnology, 10:779-783 (1992). The use of combinatorial infection and in vivo recombination as a strategy for constructing very large phage libraries is described in Waterhouse et al., Nuc. Acids Res., 21:2265-2266 (1993). Thus, these techniques are viable alternatives to traditional monoclonal antibody hybridoma methods for the generation of monoclonal antibodies.

C. Humanized Antibodies

Methods for humanizing non-human antibodies are known in the art. Preferably, a humanized antibody has one or more amino acid residues introduced into it from a source which is non-human. These non-human amino acid residues are often referred to as “import” residues, which are typically taken from an “import” variable domain. Humanization can be essentially performed by substituting the hypervariable region sequences of a non-human antibody for the corresponding sequences of a human antibody. See, e.g., Jones et al., Nature, 321:522-525 (1986); Riechmann et al., Nature, 332:323-327 (1988); and Verhoeyen et al., Science, 239:1534-1536 (1988). Accordingly, such “humanized” antibodies are chimeric antibodies (see, e.g., U.S. Pat. No. 4,816,567), wherein substantially less than an intact human variable domain has been substituted by the corresponding sequence from a non-human species. In practice, humanized antibodies are typically human antibodies in which some hypervariable region residues and possibly some framework region (FR) residues are substituted by residues from analogous sites of rodent antibodies.

The choice of human variable domains, both light and heavy, to be used in making the humanized antibodies described herein is an important consideration for reducing antigenicity. According to the so-called “best-fit” method, the sequence of the variable domain of a rodent antibody is screened against the entire library of known human variable-domain sequences. The human sequence which is closest to that of the rodent is then accepted as the human FR for the humanized antibody (see, e.g., Sims et al., J. Immunol., 151:2296 (1993); and Chothia et al., J. Mol. Biol., 196:901 (1987)). Another method uses a particular FR derived from the consensus sequence of all human antibodies of a particular subgroup of light or heavy chains. The same FR may be used for several different humanized antibodies (see, e.g., Carter et al., Proc. Natl. Acad. Sci. USA, 89:4285 (1992); and Presta et al., J. Immunol., 151:2623 (1993)).

It is also important that antibodies be humanized with retention of high affinity for the antigen and other favorable biological properties. To achieve this goal, humanized antibodies can be prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and specifically involved in influencing antigen binding.

Various forms of humanized antibodies are contemplated in accordance with the present invention. For example, the humanized antibody can be an antibody fragment, such as a Fab fragment. Alternatively, the humanized antibody can be an intact antibody, such as an intact IgA, IgG, or IgM antibody.

D. Human Antibodies

As an alternative to humanization, human antibodies can be generated. In some embodiments, transgenic animals (e.g., mice) can be produced that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci. USA, 90:2551 (1993); Jakobovits et al., Nature, 362:255-258 (1993); Bruggermann et al., Year in Immun., 7:33 (1993); and U.S. Pat. Nos. 5,591,669, 5,589,369, and 5,545,807.

Alternatively, phage display technology (see, e.g., McCafferty et al., Nature, 348:552-553 (1990)) can be used to produce human antibodies and antibody fragments in vitro, using immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B cell. Phage display can be performed in a variety of formats as described in, e.g., Johnson et al., Curr. Opin. Struct. Biol., 3:564-571 (1993). Several sources of V-gene segments can be used for phage display. See, e.g., Clackson et al., Nature, 352:624-628 (1991). A repertoire of V genes from unimmunized human donors can be constructed and antibodies to a diverse array of antigens (including self-antigens) can be isolated essentially following the techniques described in Marks et al., J. Mol. Biol., 222:581-597 (1991); Griffith et al., EMBO J., 12:725-734 (1993); and U.S. Pat. Nos. 5,565,332 and 5,573,905.

In certain instances, human antibodies can be generated by in vitro activated B cells as described in, e.g., U.S. Pat. Nos. 5,567,610 and 5,229,275.

E. Antibody Fragments

Various techniques have been developed for the production of antibody fragments. Traditionally, these fragments were derived via proteolytic digestion of intact antibodies (see, e.g., Morimoto et al., J. Biochem. Biophys. Meth., 24:107-117 (1992); and Brennan et al., Science, 229:81 (1985)). However, these fragments can now be produced directly using recombinant host cells. For example, the antibody fragments can be isolated from the antibody phage libraries discussed above. Alternatively, Fab′-SH fragments can be directly recovered from E. coli cells and chemically coupled to form F(ab′)₂ fragments (see, e.g., Carter et al., BioTechnology, 10:163-167 (1992)). According to another approach, F(ab′)₂ fragments can be isolated directly from recombinant host cell culture. Other techniques for the production of antibody fragments will be apparent to those skilled in the art. In other embodiments, the antibody of choice is a single chain Fv fragment (scFv). See, e.g., PCT Publication No. WO 93/16185; and U.S. Pat. Nos. 5,571,894 and 5,587,458. The antibody fragment may also be a linear antibody as described, e.g., in U.S. Pat. No. 5,641,870. Such linear antibody fragments may be monospecific or bispecific.

F. Bispecific Antibodies

Bispecific antibodies are antibodies that have binding specificities for at least two different epitopes. Exemplary bispecific antibodies may bind to two different epitopes of the same polypeptide of interest. Other bispecific antibodies may combine a binding site for the polypeptide of interest with binding site(s) for one or more additional antigens. Bispecific antibodies can be prepared as full-length antibodies or antibody fragments (e.g., F(ab′)₂ bispecific antibodies).

Methods for making bispecific antibodies are known in the art. Traditional production of full-length bispecific antibodies is based on the co-expression of two immunoglobulin heavy chain-light chain pairs, where the two chains have different specificities (see, e.g., Millstein et al., Nature, 305:537-539 (1983)). Because of the random assortment of immunoglobulin heavy and light chains, these hybridomas (quadromas) produce a potential mixture of 10 different antibody molecules, of which only one has the correct bispecific structure. Purification of the correct molecule is usually performed by affinity chromatography. Similar procedures are disclosed in PCT Publication No. WO 93/08829 and Traunecker et al., EMBO J., 10:3655-3659 (1991).

According to a different approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2, and CH3 regions. It is preferred to have the first heavy chain constant region (CH1) containing the site necessary for light chain binding present in at least one of the fusions. DNA encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains into one expression vector when the expression of at least two polypeptide chains in equal ratios results in high yields or when the ratios are of no particular significance.

In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. This asymmetric structure facilitates the separation of the desired bispecific compound from unwanted immunoglobulin chain combinations, as the presence of an immunoglobulin light chain in only one half of the bispecific molecule provides for a facile way of separation. See, e.g., PCT Publication No. WO 94/04690 and Suresh et al., Meth. Enzymol., 121:210 (1986).

According to another approach described in U.S. Pat. No. 5,731,168, the interface between a pair of antibody molecules can be engineered to maximize the percentage of heterodimers which are recovered from recombinant cell culture. The preferred interface comprises at least a part of the CH3 domain of an antibody constant domain. In this method, one or more small amino acid side-chains from the interface of the first antibody molecule are replaced with larger side chains (e.g., tyrosine or tryptophan). Compensatory “cavities” of identical or similar size to the large side-chain(s) are created on the interface of the second antibody molecule by replacing large amino acid side-chains with smaller ones (e.g., alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Heteroconjugate antibodies can be made using any convenient cross-linking method. Suitable cross-linking agents and techniques are well-known in the art, and are disclosed in, e.g., U.S. Pat. No. 4,676,980.

Suitable techniques for generating bispecific antibodies from antibody fragments are also known in the art. For example, bispecific antibodies can be prepared using chemical linkage. In certain instances, bispecific antibodies can be generated by a procedure in which intact antibodies are proteolytically cleaved to generate F(ab′)₂ fragments (see, e.g., Brennan et al., Science, 229:81 (1985)). These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody.

In some embodiments, Fab′-SH fragments can be directly recovered from E. coli and chemically coupled to form bispecific antibodies. For example, a fully humanized bispecific antibody F(ab′)₂ molecule can be produced by the methods described in Shalaby et al., J. Exp. Med., 175: 217-225 (1992). Each Fab′ fragment was separately secreted from E. coli and subjected to directed chemical coupling in vitro to form the bispecific antibody.

Various techniques for making and isolating bispecific antibody fragments directly from recombinant cell culture have also been described. For example, bispecific antibodies have been produced using leucine zippers. See, e.g., Kostelny et al., J. Immunol., 148:1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA, 90:6444-6448 (1993) has provided an alternative mechanism for making bispecific antibody fragments. The fragments comprise a heavy chain variable domain (VH) connected to a light chain variable domain (VL) by a linker which is too short to allow pairing between the two domains on the same chain. Accordingly, the VH and VL domains of one fragment are forced to pair with the complementary VL and VH domains of another fragment, thereby forming two antigen binding sites. Another strategy for making bispecific antibody fragments by the use of single-chain Fv (sFv) dimers is described in Gruber et al., J. Immunol., 152:5368 (1994).

Antibodies with more than two valencies are also contemplated. For example, trispecific antibodies can be prepared. See, e.g., Tutt et al., J. Immunol., 147:60 (1991).

G. Antibody Purification

When using recombinant techniques, antibodies can be produced inside an isolated host cell, in the periplasmic space of a host cell, or directly secreted from a host cell into the medium. If the antibody is produced intracellularly, the particulate debris is first removed, for example, by centrifugation or ultrafiltration. Carter et al., BioTech., 10:163-167 (1992) describes a procedure for isolating antibodies which are secreted into the periplasmic space of E. coli. Briefly, cell paste is thawed in the presence of sodium acetate (pH 3.5), EDTA, and phenylmethylsulfonylfluoride (PMSF) for about 30 min. Cell debris can be removed by centrifugation. Where the antibody is secreted into the medium, supernatants from such expression systems are generally concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The antibody composition prepared from cells can be purified using, for example, hydroxylapatite chromatography, gel electrophoresis, dialysis, and affinity chromatography. The suitability of protein A as an affinity ligand depends on the species and isotype of any immunoglobulin Fc domain that is present in the antibody. Protein A can be used to purify antibodies that are based on human γ1, γ2, or γ4 heavy chains (see, e.g., Lindmark et al., J. Immunol. Meth., 62:1-13 (1983)). Protein G is recommended for all mouse isotypes and for human γ3 (see, e.g., Guss et al., EMBO J., 5:1567-1575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a CH3 domain, the Bakerbond ABX™ resin (J. T. Baker; Phillipsburg, N.J.) is useful for purification. Other techniques for protein purification such as fractionation on an ion-exchange column, ethanol precipitation, reverse phase HPLC, chromatography on silica, chromatography on heparin SEPHAROSE™, chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between about 2.5-4.5, preferably performed at low salt concentrations (e.g., from about 0-0.25 M salt).

One of skill in the art will appreciate that any binding molecule having a function similar to an antibody, e.g., a binding molecule or binding partner which is specific for one or more analytes of interest in a sample, can also be used in the methods and compositions of the present invention. Examples of suitable antibody-like molecules include, but are not limited to, domain antibodies, unibodies, nanobodies, shark antigen reactive proteins, avimers, adnectins, anticalms, affinity ligands, phylomers, aptamers, affibodies, trinectins, and the like.

### IX. Methods of Administration

According to the methods of the present invention, the anticancer drugs described herein are administered to a subject by any convenient means known in the art. The methods of the present invention can be used to select a suitable anticancer drug or combination of anticancer drugs for the treatment of a tumor, e.g., non-small cell lung cancer tumor, in a subject. The methods of the present invention can also be used to identify the response of a tumor, e.g., non-small cell lung cancer tumor, in a subject to treatment with an anticancer drug or combination of anticancer drugs. In addition, the methods of the present invention can be used to predict the response of a subject having a tumor, e.g., non-small cell lung cancer tumor, to treatment with an anticancer drug or combination of anticancer drugs. The methods of the present invention can also be used to monitor the status of a tumor, e.g., non-small cell lung cancer tumor, in a subject or to monitor how a patient with the tumor is responding to treatment with an anticancer drug or combination of anticancer drugs. One skilled in the art will appreciate that the anticancer drugs described herein can be administered alone or as part of a combined therapeutic approach with conventional chemotherapy, radiotherapy, hormonal therapy, immunotherapy, and/or surgery.

In certain embodiments, the anticancer drug comprises an anti-signaling agent (i.e., a cytostatic drug) such as a monoclonal antibody or a tyrosine kinase inhibitor; an anti-proliferative agent; a chemotherapeutic agent (i.e., a cytotoxic drug); a hormonal therapeutic agent; a radiotherapeutic agent; a vaccine; and/or any other compound with the ability to reduce or abrogate the uncontrolled growth of aberrant cells such as cancerous cells. In some embodiments, the subject is treated with one or more anti-signaling agents, anti-proliferative agents, and/or hormonal therapeutic agents in combination with at least one chemotherapeutic agent. Exemplary monoclonal antibodies, tyrosine kinase inhibitors, anti-proliferative agents, chemotherapeutic agents, hormonal therapeutic agents, radiotherapeutic agents, and vaccines are described above.

In some embodiments, the anticancer drugs described herein can be co-administered with conventional immunotherapeutic agents including, but not limited to, immunostimulants (e.g., Bacillus Calmette-Guérin (BCG), levamisole, interleukin-2, alpha-interferon, etc.), immunotoxins (e.g., anti-CD33 monoclonal antibody-calicheamicin conjugate, anti-CD22 monoclonal antibody-pseudomonas exotoxin conjugate, etc.), and radioimmunotherapy (e.g., anti-CD20 monoclonal antibody conjugated to ¹¹¹In, ⁹⁰Y, or ¹³¹I, etc.).

Anticancer drugs can be administered with a suitable pharmaceutical excipient as necessary and can be carried out via any of the accepted modes of administration. Thus, administration can be, for example, oral, buccal, sublingual, gingival, palatal, intravenous, topical, subcutaneous, transcutaneous, transdermal, intramuscular, intra joint, parenteral, intra-arteriole, intradermal, intraventricular, intracranial, intraperitoneal, intravesical, intrathecal, intralesional, intranasal, rectal, vaginal, or by inhalation. By “co-administer” it is meant that an anticancer drug is administered at the same time, just prior to, or just after the administration of a second drug (e.g., another anticancer drug, a drug useful for reducing the side-effects associated with anticancer drug therapy, a radiotherapeutic agent, a hormonal therapeutic agent, an immunotherapeutic agent, etc.).

A therapeutically effective amount of an anticancer drug may be administered repeatedly, e.g., at least 2, 3, 4, 5, 6, 7, 8, or more times, or the dose may be administered by continuous infusion. The dose may take the form of solid, semi-solid, lyophilized powder, or liquid dosage forms, such as, for example, tablets, pills, pellets, capsules, powders, solutions, suspensions, emulsions, suppositories, retention enemas, creams, ointments, lotions, gels, aerosols, foams, or the like, preferably in unit dosage forms suitable for simple administration of precise dosages.

As used herein, the term “unit dosage form” refers to physically discrete units suitable as unitary dosages for human subjects and other mammals, each unit containing a predetermined quantity of an anticancer drug calculated to produce the desired onset, tolerability, and/or therapeutic effects, in association with a suitable pharmaceutical excipient (e.g., an ampoule). In addition, more concentrated dosage forms may be prepared, from which the more dilute unit dosage forms may then be produced. The more concentrated dosage forms thus will contain substantially more than, e.g., at least 1, 2, 3, 4, 5, 6, 7, 8, 9, 10, or more times the amount of the anticancer drug.

Methods for preparing such dosage forms are known to those skilled in the art (see, e.g., REMINGTON'S PHARMACEUTICAL SCIENCES, 18TH ED., Mack Publishing Co., Easton, Pa. (1990)). The dosage forms typically include a conventional pharmaceutical carrier or excipient and may additionally include other medicinal agents, carriers, adjuvants, diluents, tissue permeation enhancers, solubilizers, and the like. Appropriate excipients can be tailored to the particular dosage form and route of administration by methods well known in the art (see, e.g., REMINGTON'S PHARMACEUTICAL SCIENCES, supra).

Examples of suitable excipients include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline, syrup, methylcellulose, ethylcellulose, hydroxypropylmethylcellulose, and polyacrylic acids such as Carbopols, e.g., Carbopol 941, Carbopol 980, Carbopol 981, etc. The dosage forms can additionally include lubricating agents such as talc, magnesium stearate, and mineral oil; wetting agents; emulsifying agents; suspending agents; preserving agents such as methyl-, ethyl-, and propyl-hydroxy-benzoates (i.e., the parabens); pH adjusting agents such as inorganic and organic acids and bases; sweetening agents; and flavoring agents. The dosage forms may also comprise biodegradable polymer beads, dextran, and cyclodextrin inclusion complexes.

For oral administration, the therapeutically effective dose can be in the form of tablets, capsules, emulsions, suspensions, solutions, syrups, sprays, lozenges, powders, and sustained-release formulations. Suitable excipients for oral administration include pharmaceutical grades of mannitol, lactose, starch, magnesium stearate, sodium saccharine, talcum, cellulose, glucose, gelatin, sucrose, magnesium carbonate, and the like.

In some embodiments, the therapeutically effective dose takes the form of a pill, tablet, or capsule, and thus, the dosage form can contain, along with an anticancer drug, any of the following: a diluent such as lactose, sucrose, dicalcium phosphate, and the like; a disintegrant such as starch or derivatives thereof; a lubricant such as magnesium stearate and the like; and a binder such a starch, gum acacia, polyvinylpyrrolidone, gelatin, cellulose and derivatives thereof. An anticancer drug can also be formulated into a suppository disposed, for example, in a polyethylene glycol (PEG) carrier.

Liquid dosage forms can be prepared by dissolving or dispersing an anticancer drug and optionally one or more pharmaceutically acceptable adjuvants in a carrier such as, for example, aqueous saline (e.g., 0.9% w/v sodium chloride), aqueous dextrose, glycerol, ethanol, and the like, to form a solution or suspension, e.g., for oral, topical, or intravenous administration. An anticancer drug can also be formulated into a retention enema.

For topical administration, the therapeutically effective dose can be in the form of emulsions, lotions, gels, foams, creams, jellies, solutions, suspensions, ointments, and transdermal patches. For administration by inhalation, an anticancer drug can be delivered as a dry powder or in liquid form via a nebulizer. For parenteral administration, the therapeutically effective dose can be in the form of sterile injectable solutions and sterile packaged powders. Preferably, injectable solutions are formulated at a pH of from about 4.5 to about 7.5.

The therapeutically effective dose can also be provided in a lyophilized form. Such dosage forms may include a buffer, e.g., bicarbonate, for reconstitution prior to administration, or the buffer may be included in the lyophilized dosage form for reconstitution with, e.g., water. The lyophilized dosage form may further comprise a suitable vasoconstrictor, e.g., epinephrine. The lyophilized dosage form can be provided in a syringe, optionally packaged in combination with the buffer for reconstitution, such that the reconstituted dosage form can be immediately administered to a subject.

A subject can also be monitored at periodic time intervals to assess the efficacy of a certain therapeutic regimen. For example, the activation states of certain signal transduction molecules may change based on the therapeutic effect of treatment with one or more of the anticancer drugs described herein. The subject can be monitored to assess response and understand the effects of certain drugs or treatments in an individualized approach. Additionally, subjects who initially respond to a specific anticancer drug or combination of anticancer drugs may become refractory to the drug or drug combination, indicating that these subjects have developed acquired drug resistance. These subjects can be discontinued on their current therapy and an alternative treatment prescribed in accordance with the methods of the present invention.

In certain aspects, the methods described herein can be used in conjunction with panels of gene expression markers that predict the likelihood of stomach cancer prognosis and/or recurrence in various populations. These gene panels can be useful for identifying individuals who are unlikely to experience recurrence and, thus, unlikely to benefit from adjuvant chemotherapy. The expression panels can be used to identify individuals who can safely avoid adjuvant chemotherapy, without negatively affecting disease-free and overall survival outcomes. Suitable systems include, but are not limited to, Oncotype DX™, which is a 21-gene panel from Genomic Health, Inc.; MammaPrint,® which is a 70-gene panel from Agendia; and a 76-gene panel from Veridex.

In addition, in certain other aspects, the methods described herein can be used in conjunction with panels of gene expression markers that identify the original tumors for cancers of unknown primary (CUP). These gene panels can be useful in identifying patients with metastatic cancer who would benefit from therapy consistent with that given to patients diagnosed initially with lung cancer. Suitable systems include, but are not limited to, the Aviara CancerTYPE ID assay, an RT-PCR-based expression assay that measures 92 genes to identify the primary site of origin for 39 tumor types; and the Pathwork® Tissue of Origin Test, which measures the expression of more than 1600 genes on a microarray and compares a tumor's gene expression “signature” against those of 15 known tissue types.”

### X. Examples

The following examples are offered to illustrate, but not to limit, the claimed invention.

Examples 1 and 2 of PCT Application No. PCT/US2010/042182, filed Jul. 15, 2010, are herein incorporated by reference in their entirety for all purposes.

### Example 1

### Single Detection Microarray ELISA with Tyramide Signal Amplification

This example illustrates a multiplex, high-throughput, single detection microarray ELISA having superior dynamic range that is suitable for analyzing the expression level or activation level of signal transduction molecules in samples such as whole blood (e.g., rare circulating cells) or tumor tissue (e.g., fine needle aspirate):
1) Capture antibody was printed on a 16-pad FAST slide (Whatman Inc.; Florham Park, N.J.) with a 2-fold serial dilution.
2) After drying overnight, the slide was blocked with Whatman blocking buffer.
3) 80 μl of cell lysate was added onto each pad with a 10-fold serial dilution. The slide was incubated for two hours at room temperature.
4) After six washes with TBS-Tween, 80 μl of biotin-labeled detection antibody (e.g., a monoclonal antibody recognizing phosphorylated c-Met or a monoclonal antibody recognizing c-Met regardless of activation state) was incubated for two hours at room temperature.
5) After six washes, streptavidin-labeled horseradish peroxidase (SA-HRP) was added and incubated for 1 hour to allow the SA-HRP to bind to the biotin-labeled detection antibody.
6) For signal amplification, 80 μl of biotin-tyramide at 5 μg/ml was added and reacted for 15 minutes. The slide was washed six times with TBS-Tween, twice with 20% DMSO/TBS-Tween, and once with TBS.
7) 80 μl of SA-Alexa 555 was added and incubated for 30 minutes. The slide was then washed twice, dried for 5 minutes, and scanned on a microarray scanner (Perkin-Elmer, Inc.; Waltham, Mass.).

### Example 2

### Proximity Dual Detection Microarray ELISA with Tyramide Signal Amplification

This example illustrates a multiplex, high-throughput, proximity dual detection microarray ELISA having superior dynamic range (e.g., Collaborative Enzyme Enhanced Reactive ImmunoAssay (CEER)) that is suitable for analyzing the expression level and/or activation level of signal transduction molecules in samples such as whole blood (e.g., rare circulating cells) or tumor tissue (e.g., fine needle aspirate):
1) Capture antibody was printed on a 16-pad FAST slide (Whatman Inc.) with a serial dilution ranging from 1 mg/ml to 0.004 mg/ml.
2) After drying overnight, the slide was blocked with Whatman blocking buffer.
3) 80 μl of A431 cell lysate was added onto each pad with a 10-fold serial dilution. The slide was incubated for two hours at room temperature.
4) After six washes with TBS-Tween, 80 μl of detection antibodies for the proximity assay diluted in TBS-Tween/2% BSA/1% FBS was added to the slides. The incubation was for 2 hours at room temperature.
   a) As a non-limiting example, the detection antibodies can comprise the following: (i) a monoclonal antibody recognizing c-Met regardless of its activation state that is directly conjugated to glucose oxidase (GO); and (ii) either a monoclonal antibody recognizing phosphorylated c-Met that is directly conjugated to horseradish peroxidase (HRP) or a monoclonal antibody recognizing c-Met regardless of its activation state at a different epitope than the first detection antibody that is directly conjugated to HRP.
   b) Alternatively, the detection step can utilize a biotin-conjugate of the second detection antibody. In these embodiments, after six washes, an additional sequential step of incubation with streptavidin-HRP for 1 hour is included.
   c) Alternatively, the detection step can utilize an oligonucleotide-mediated glucose oxidase (GO) conjugate of the first detection antibody. Either the directly conjugated or the biotin-steptavidin (SA) linked conjugate of HRP to the second detection antibody can be used.
5) For signal amplification, 80 μl of biotin-tyramide at 5 μg/ml was added and reacted for 15 min. The slide was washed six times with TBS-Tween, twice with 20% DMSO/TBS-Tween, and once with TBS.
6) 80 μl of SA-Alexa 555 was added and incubated for 30 min. The slide was then washed twice, dried for 5 minutes, and scanned on a microarray scanner (Perkin-Elmer, Inc.).

### Example 3

### Generation of Activation Profiles for Drug Selection

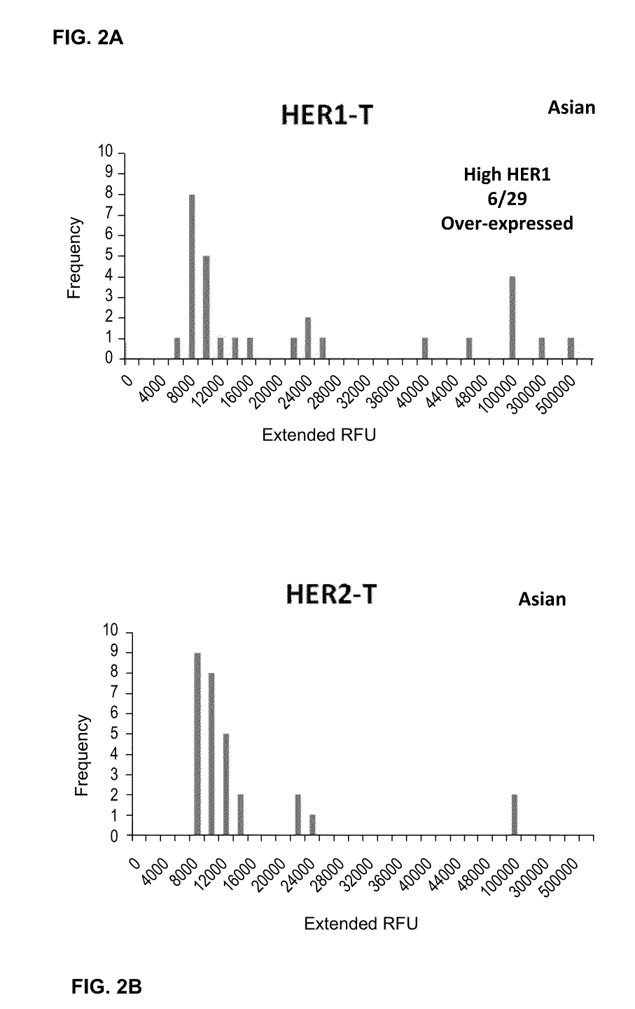

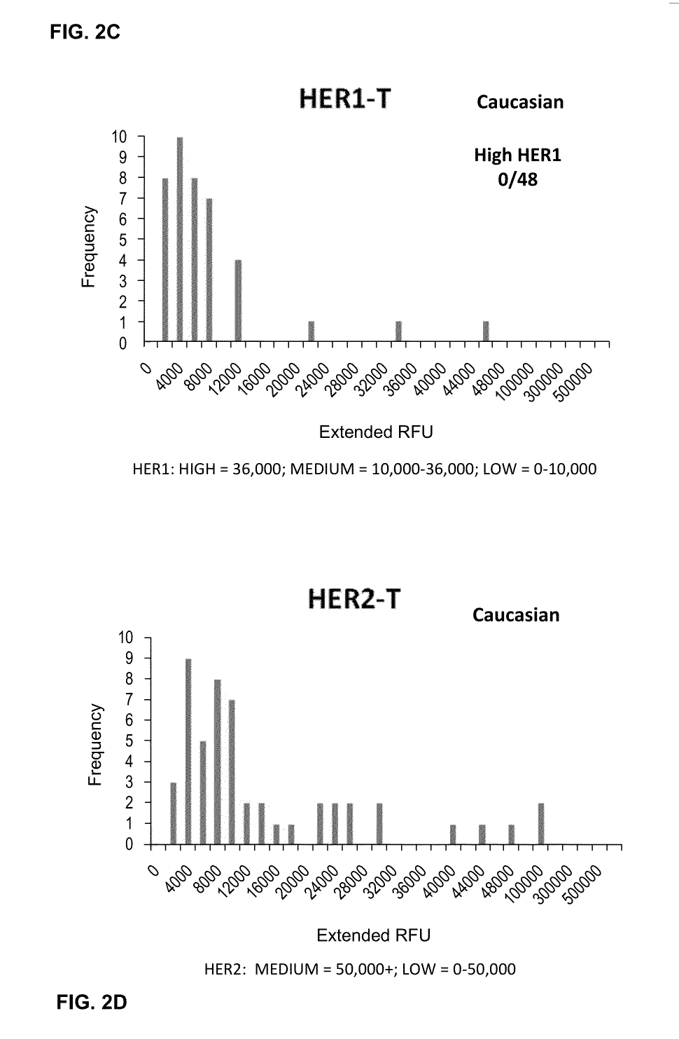

The methods and compositions of the present invention can be applied for drug selection for cancer treatment. A typical protocol entails the generation of two profiles, a reference activation profile and a test activation profile, which are then compared to determine the efficacy of a particular drug treatment regimen (see, FIG. 2 of PCT Application No. PCT/US2010/042182, filed Jul. 15, 2010, which is herein incorporated by reference in its entirety for all purposes).

### Reference Activation Profile

To derive a reference activation profile, a tumor tissue, blood or fine needle aspirate (FNA) sample is obtained from a patient having a specific type of cancer (e.g., lung cancer) prior to anticancer drug treatment. Tumor cells are isolated from the tumor, blood, or FNA sample. The isolated cells can be stimulated in vitro with one or more growth factors. The stimulated cells are then lysed to produce a cellular extract. The cellular extract is applied to an addressable array containing a dilution series of a panel of capture antibodies specific for signal transduction molecules whose activation states may be altered in the patient's type of cancer. Single detection or proximity assays are performed using the appropriate detection antibodies (e.g., activation state-independent antibodies and/or activation state-dependent antibodies) to determine the activation state of each signal transduction molecule of interest. The “Pathway Selection” table shown in Table 2 is particularly useful for selecting which activation states to detect based upon the patient's type of cancer. For example, one patient may have a type of cancer that displays the activation states of the EGFR pathway set forth in “Pathway 1” of Table 2. Alternatively, another patient may have another type of cancer that displays the activation states of the EGFR pathway set forth in “Pathway 2” of Table 2. A reference activation profile is thus generated providing the activation states of signal transduction molecules in the patient's cancer in the absence of any anticancer drugs.

### Test Activation Profile

To obtain a test activation profile, a second tumor tissue, blood or FNA sample is obtained from the patient having the specific type of cancer (e.g., lung cancer) either prior to anticancer drug treatment or after administration of an anticancer drug (e.g., at any time throughout the course of cancer treatment). Tumor cells are isolated from the tumor, blood, or FNA sample. If isolated cells are obtained from a patient who has not received treatment with an anticancer drug, the isolated cells are incubated with anticancer drugs which target one or more of the activated signal transduction molecules determined from the reference activation profile described above. The “Drug Selection” table (Table 1) is particularly useful for selecting appropriate anticancer drugs that are either approved or in clinical trials which inhibit specific activated target signal transduction molecules. For example, if it is determined from the reference activation profile that EGFR is activated, then the cells can be incubated with one or more of the drugs listed in column “A” or “B” of Table 1. The isolated cells can then be stimulated in vitro with one or more growth factors. The isolated cells are then lysed to produce a cellular extract. The cellular extract is applied to the addressable array and proximity assays are performed to determine the activation state of each signal transduction molecule of interest. A test activation profile for the patient is thus generated providing the activation states of signal transduction molecules in the patient's cancer in the presence of specific anticancer drugs.

### Drum Selection

The anticancer drugs are determined to be suitable or unsuitable for treatment of the patient's cancer by comparing the test activation profile to the reference activation profile. For example, if drug treatment causes most or all of the signal transduction molecules to be substantially less activated than in the absence of the drugs, e.g., a change from strong activation without the drugs to weak or very weak activation with the drugs, then the treatment is determined to be suitable for the patient's cancer. In such instances, treatment is either initiated with the suitable anticancer drug in a patient who has not received drug therapy or subsequent treatment is continued with the suitable anticancer drug in a patient already receiving the drug. However, if the drug treatment is deemed unsuitable for treatment of the patient's cancer, different drugs are selected and used to generate a new test activation profile, which is then compared to the reference activation profile. In such instances, treatment is either initiated with a suitable anticancer drug in a patient who has not received drug therapy or subsequent treatment is changed to a suitable anticancer drug in a patient currently receiving the unsuitable drug.

### Example 4

### Method to Detect c-Met Activation for Anticancer Drug Therapy Selection

This example illustrates the use of the multiplexed protein microarray platform described herein to identify patients who would respond to anti-c-Met inhibitors and to identify patients who would benefit from a combination of anti-c-Met inhibitors with other targeted agents.

A wide variety of human malignancies exhibit sustained c-Met stimulation, overexpression, or mutation, including carcinomas of the breast, liver, lung, ovary, kidney, and thyroid. Notably, activating mutations in c-Met have been positively identified in patients with a particular hereditary form of papillary renal cancer, directly implicating c-Met in human tumorigenesis. Aberrant signaling of the c-Met signaling pathway due to dysregulation of the c-Met receptor or overexpression of its ligand, hepatocyte growth factor (HGF), has been associated with an aggressive phenotype.

Extensive evidence that c-Met signaling is involved in the progression and spread of several cancers and an enhanced understanding of its role in disease have generated considerable interest in c-Met and HGF as major targets in cancer drug development. This has led to the development of a variety of c-Met pathway antagonists with potential clinical applications. The three main approaches of pathway-selective anticancer drug development have included antagonism of ligand/receptor interaction, inhibition of the tyrosine kinase catalytic activity, and blockade of the receptor/effector interaction.

Several c-Met antagonists are now under clinical investigation. Preliminary clinical results of several of these agents, including both monoclonal antibodies and small molecule tyrosine kinase inhibitors, have been encouraging. Interestingly, patients with c-Met amplification do not respond to tyrosine kinase inhibitors. Several multi-targeted therapies have also been under investigation in the clinic and have demonstrated promise, particularly with regard to tyrosine kinase inhibition.

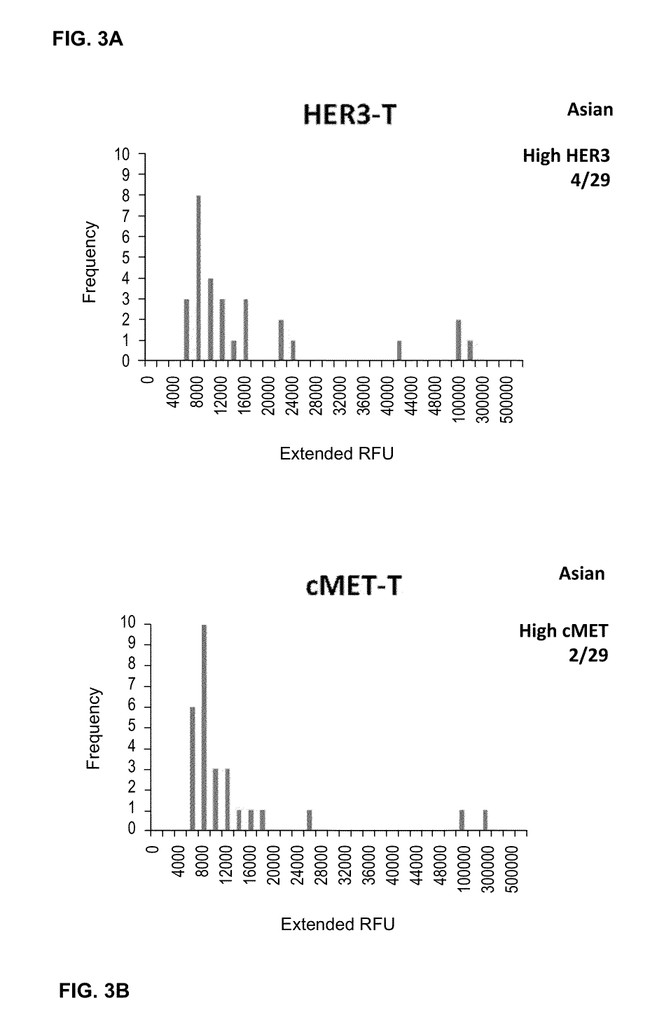

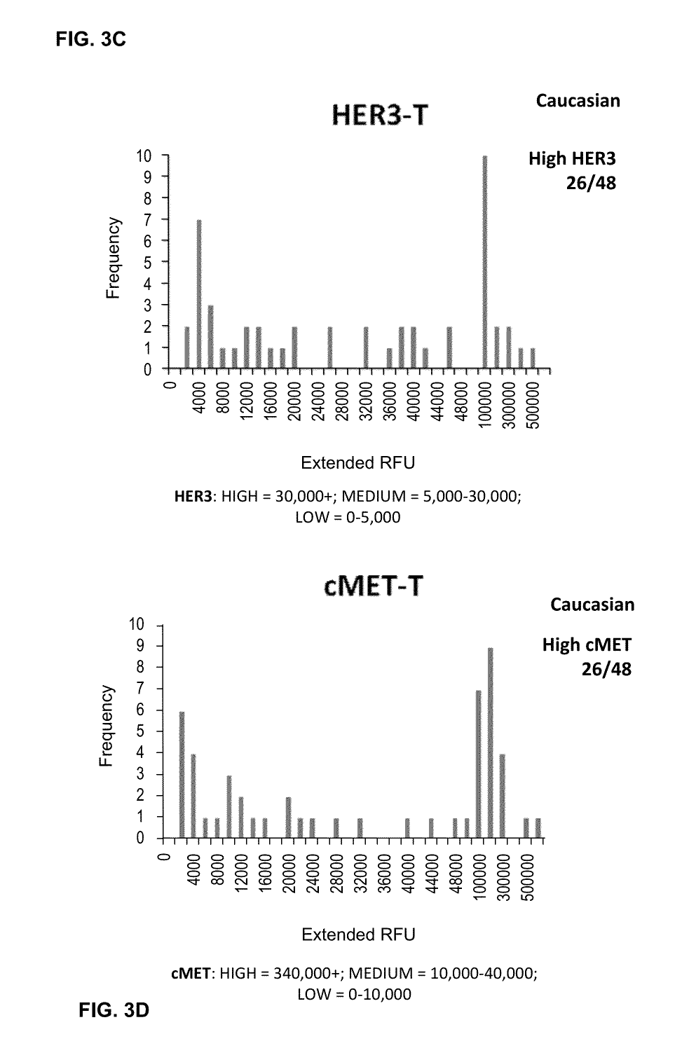

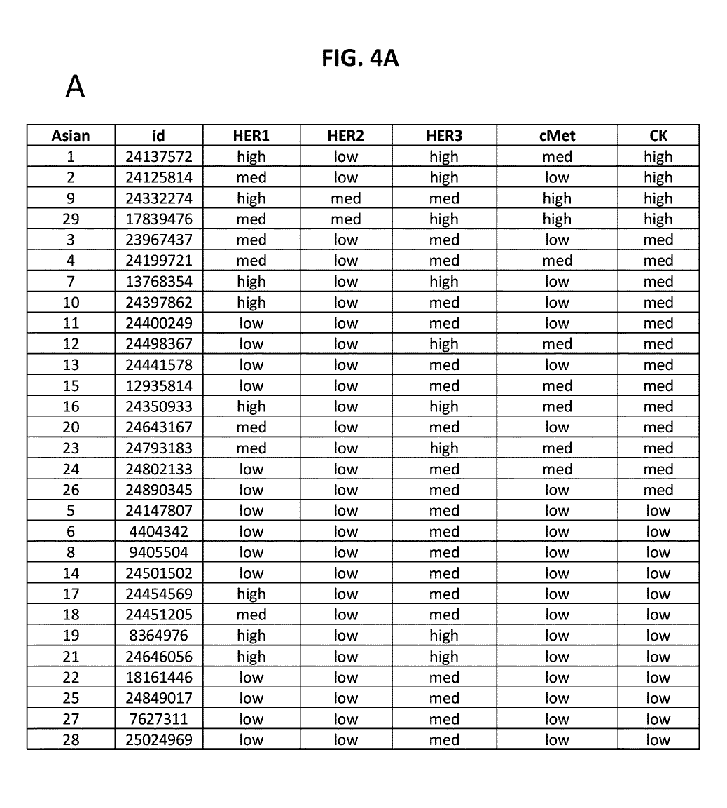

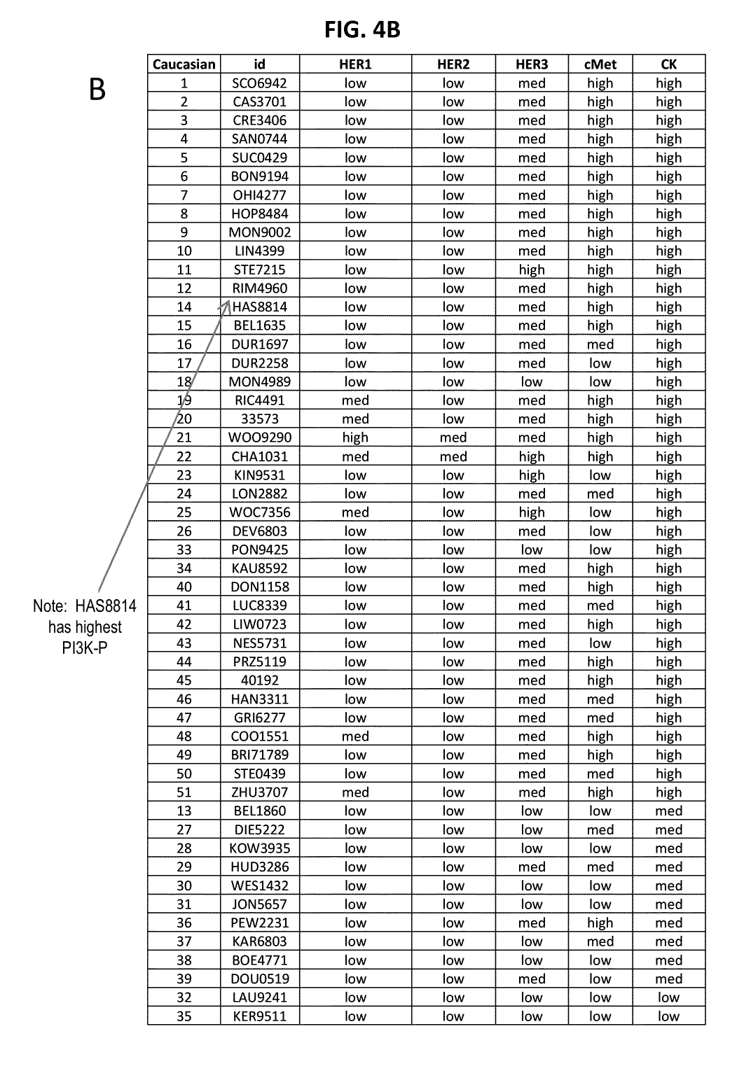

The c-Met receptor tyrosine kinase can be overexpressed in many malignancies and is important in biological and biochemical functions. Activation of the c-Met receptor can lead to increased cell growth, invasion, angiogenesis, and metastasis. Amplification and/or activation mutations within the tyrosine kinase domain, juxtamembrane domain, or semaphorin domain have been identified for c-Met. A number of therapeutic strategies have been employed to inhibit c-Met. Several clinical trials are investigating c-Met and its ligand, hepatocyte growth factor, for various malignancies. As such, methods for profiling c-Met expression and/or phosphorylation in cancer cells found in a patient's tumor tissue, whole blood or fine needle aspirate (FNA) sample provide valuable insight into the overall disease pathogenesis, and therefore lead to better anticancer therapy selection. See, e.g., FIGS. 3 and 4 of PCT Publication No. WO 2011/008990, which are herein incorporated by reference in their entirety for all purposes.

The multiplexed protein microarray platform described herein (e.g., CEER) utilizes the formation of a unique immuno-complex requiring the co-localization of two detector enzyme-conjugated-antibodies once target proteins are captured on the microarray surface. The channeling events between two detector enzymes (glucose oxidase (GO) and horseradish peroxidase (HRP)) in proximity enables the profiling of a receptor tyrosine kinase (RTK) such as c-Met with extreme sensitivity. The analytical specificity is greatly enhanced given the requirement for simultaneous binding of three different antibodies. In particular, the multiplexed proximity assay is based on (1) a multiplexed protein microarray platform combined with (2) a triple-antibody-enzyme channeling signal amplification process. The unique and novel design is provided by the triple-antibody enzyme approach that confers ultra-high sensitivity while preserving specificity:
(1) The selected target is captured by target-specific antibodies printed in serial dilutions on a microarray surface. This format requires a co-localization of two additional detector-antibodies linked with enzymes for subsequent channeling events per each target protein bound (see, e.g., FIG. 5 of PCT Publication No. WO 2011/008990, which is herein incorporated by reference in its entirety for all purposes).
(2) The immuno-complex formed by the initial target binding by capture antibodies and the secondary binding of GO (TON of 10⁵/min) conjugated antibodies that recognize an alternate epitope on the captured target molecules can produce H₂O₂ in the presence of the GO substrate, glucose.
(3) The target-specific local influx of H₂O₂ is then utilized by phospho-peptide-specific antibodies conjugated with HRP (TON of 10⁴/min) that bind to the phosphorylated peptide on the captured targets, hence amplifying target specific signals. Specificity for the detection of phosphorylated targets is greatly increased through the collaborative immuno-detection and amplification process given the requirement for simultaneous binding of three different types of antibodies. The detection and quantification of as few as ˜2-3×10⁴ phosphorylation events is routinely achieved by this method, bringing its detection to a “single” cell level. In certain instances, this collaborative immunoassay configuration can be further applied to investigate protein interactions and activations.

Table 3 illustrates the percentage of patients with primary tumors having c-Met expression, mutation, or activation. Interestingly, patients with MET amplification in gastric cancer do not respond to c-Met inhibitors.

TABLE 3

| | MET Expression | MET Mutation | Met Amplification |
|---|---|---|---|
| Tumor Type | (% patients) | (% patients) | (% patients) |
| Brain | 54-88 | 0-9 | 9-20 |
| Head & Neck | 52-68 | 11-27 | n/a |
| Mesothelioma | 74-100 | 0 | n/a |
| Lung | 41-72 | 8-13 | 0 |
| Thyroid | 40-91 | 6-10 | n/a |
| Breast | 25-60 | 0 | n/a |
| Renal cell | 54-87 | 13-100 | Trisomy 7 |
| Hepatoma | 68 | 0-30 | n/a |
| Colon | 55-78 | 0 | 4-89 |
| Ovarian | 64 | 0-4 | 0 |
| Gastric | 75-90 | n/a | 10-20 |
| Melanoma | 17-30 | 0 | n/a |

c-Met has been demonstrated to interact with and phosphorylate kinases such as RON, EGFR, HER2, HER3, PI3K, and SHC. c-Met may interact with other kinases as well, e.g., p95HER2, IGF-1R, c-KIT, and others. The multiplexed protein microarray described herein may be performed to interrogate the status of one or more of these kinases and their pathways using the a patient's tumor tissue, whole blood (e.g., circulating tumor cells) or FNA sample. The results of the assay enable the determination of the correct anticancer therapy for each individual patient.

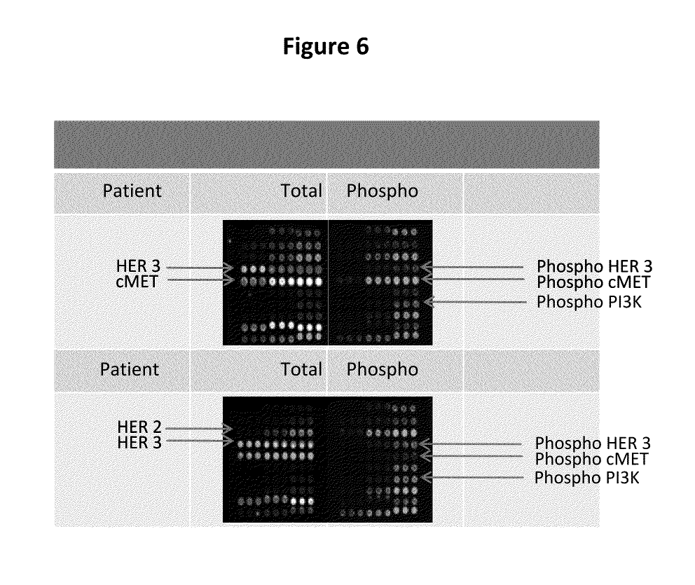

FIG. 6 of PCT Publication No. WO 2011/008990, which is herein incorporated by reference in its entirety for all purposes, illustrates an exemplary addressable array of the invention for determining the expression and/or activation status of the following markers: c-MET, HER1/ErbB1, HER2/ErbB2, p95ErbB2, HER3/ErbB3, IGF-1R, RON, c-KIT, PI3K, SHC, VEGFR1, VEGFR2, and VEGFR3. Interrogation of these receptor tyrosine kinases and their pathways using the proximity assay microarray format advantageously enables the prediction of a patient's response to a particular c-Met inhibitor therapy. As a non-limiting example, patients who respond to XL-880 will have activated c-MET and VEGFR2, while non-responders will have a combination of RTKs activated. Importantly, the proximity assay microarray platform can also be used to select the appropriate combination therapy. For example, patients with activated c-MET, VEGFR2, and EGFR should be treated with a combination of Iressa+XL880, while patients with activated c-MET, VEGFR2, ErbB1, ErbB2, ErbB3, and p95ErbB2 should be treated with Tykerb+XL880.

The multiplexed proximity-mediated platform advantageously provides single cell level sensitivity for detecting the activation of RTKs such as c-Met in a limited amount of sample. As such, tumor tissue, circulating tumor cell (CTC) and/or mFNA samples obtained from patients with metastatic cancer can be profiled to provide valuable information for tailoring therapy and impacting clinical practice.

### Example 5

### Serial Profiling and Monitoring of Cancer Therapy

The expression/activation profiling of kinases and other signal transduction pathway molecules on a serial sampling of tumor tissues provides valuable information on changes occurring in tumor cells as a function of time and therapies. This temporal profiling of tumor progression enables clinicians to monitor rapidly evolving cancer signatures in each patient. This example illustrates a novel and robust assay to detect the level of expression and the degree of phosphorylation of receptor tyrosine kinase (RTK) pathways implicated in cancer and demonstrates the advantages of using such a therapy-guiding diagnostic system with single cell level sensitivity. The assay generally relies on samples such as tumor tissue (e.g., lung tumor), fine needle aspirates (FNAs) and blood and achieves high sensitivity and specificity for interrogating the limited amount of cancer cells obtained from such samples.

The multiplexed protein microarray platform described herein (e.g., CEER) can be used to interrogate the expression/activation of kinases and other signal transduction pathway molecules associated with a malignancy involving aberrant c-Met signaling (e.g., lung cancer). As such, methods for profiling cancer markers in cancer cells found in a patient's tumor tissue, whole blood or fine needle aspirate (FNA) sample provide valuable insight into the overall disease pathogenesis, and therefore lead to better anticancer therapy selection.

As a non-limiting example, tumor tissue, whole blood, or FNA samples may be obtained from patients with lung cancer for RTK pathway interrogation using the proximity assays described herein (e.g., CEER). Alternatively, samples for pathway analysis can be obtained from frozen tissues either by sectioning or performing a frozen FNA procedure. In certain instances, tissue sectioning is the preferred method for frozen specimens for subsequent profile analysis, while the relatively non-invasive FNA procedure is the preferred method for obtaining samples from patients (and xenografts) in a clinical environment.

Frozen tissue samples may be collected by the following methods:

### Option #1. Tissue Section Collection:

1. Keep a plastic weighing boat on dry ice, in which sample cutting will take place.
   a. To chill the materials, keep razor blades or microtome blades, fine forceps, and pre-labeled sample collection vials on dry ice.
2. Take frozen human cancer tissues from −80° C. freezer and transfer samples immediately onto dry ice.
3. Place frozen tissue to weighting boat on dry ice, cut small pieces of frozen tissue (10 μm section×3) using razor blade or microtome blade, and transfer the tissue into pre-chilled and pre-labeled sample collection vial using pre-chilled forceps.
4. Close cap and keep it on dry ice.
5. Place collected specimens into a double plastic bag first and then into a Styrofoam container (primary container) with adequate amount of dry ice.
   a. Use at least 6-8 pounds dry ice. Use more in the summer months.
      NOTE: Exact amount of dry ice will be determined after consulting with a shipping company.
   b. Consult with shipping company for the international shipping process for necessary permits and documentations.
   c. Do not use wet ice, or coolants (i.e., Cool Packs).
6. Make certain the requisition and sample list is placed in the box, but on the outside of the double bag.
7. Securely seal the container and label “Frozen Tissue—Do Not Thaw.”
   Option #2. FNA Prep from Frozen Tissues:
1. Take frozen human cancer tissues from −80° C. freezer and transfer sample vials immediately on dry ice.
2. Samples ready for FNA procedure should be placed on wet ice for 10 minutes to soften the tissue.
3. FNA sample collection should be performed by passing a 23 or 25 gauge needle through softened frozen tissue 5 to 10 times. Return remaining sample vial to dry ice.
4. Wipe the FNA sample collection vial lid with alcohol.
5. Frozen FNA tissues should be collected by direct injection into the collection vial containing 100 μl of “protein later solution” (Prometheus Laboratories; San Diego, Calif.). Dispense collected tissue materials by gently mixing the content.
6. Hold the FNA collection vial firmly with one hand and perform rapid finger tapping (˜15×) to ensure through cell lysis (vortex for 10 seconds if possible).
7. Place collected specimens into a double plastic bag first and then into a Styrofoam container (primary container) with Cool Packs.
   a. Consult with shipping company for the international shipping process for necessary permits and documentations.
8. Make certain the requisition and sample list is placed in the box, but on the outside of the double bag.
9. Securely seal the container and label with “Biological Specimen.”

The multiplexed proximity-mediated platform advantageously provides single cell level sensitivity for detecting the expression and/or activation of RTKs and their pathways over time to detect changes occurring in tumor cells as a function of time and therapies. This temporal profiling of tumor status, e.g., by performing a serial sampling of tumor tissue or other sample over time, enables clinicians to monitor rapidly evolving cancer signatures in each patient and provides valuable information for tailoring therapy and impacting clinical practice.

### Example 6

### Selection of Patients for Treatment After Determination of Primary Tissue of Origin by a Gene Expression Panel

Approximately 3% to 5% of all metastatic tumors are classified into the category of cancer of unknown primary (CUP). Correct diagnosis of the tissue of origin is important in treatment decisions because current therapies are based largely on anatomical site. For example, gene expression panels can be useful in identifying patients with metastatic lung cancer who would benefit from therapy consistent with that given to patients diagnosed initially with lung cancer. Suitable systems include, but are not limited to, the Rosetta Genomics CUP assay, which classifies cancers and tissues of origin through the analysis of the expression patterns of microRNAs (see, e.g., PCT Publication No. WO 08/117,278); the Aviara DX (Carlsbad, Calif.) CancerTYPE ID™ assay, an RT-PCR-based expression assay that measures 92 genes to identify the primary site of origin for 39 tumor types; and the Pathwork™ Tissue of Origin Test (Sunnyvale, Calif.), which measures the expression of more than 1600 genes on a microarray and compares a tumor's gene expression “signature” against those of 15 known tissue types. Once the patient has been identified with the lung as the tissue of primary cancer, pathway activation profiles can be used to select the appropriate targeted therapies to include in the treatment schedule.

The following protocol provides an exemplary embodiment of the present invention wherein gene expression profiling is used in conjunction with activation state profiling to select the appropriate targeted therapy or combination of targeted therapies for the treatment of a malignancy involving aberrant cMet signaling such as lung cancer:
1) Two or more glass slides with 7 μm thick sections of a tissue removed, either surgically or by fine needle biopsy, from a metastatic tumor are obtained from the patient. These cells are fixed in formalin and embedded in paraffin (FFPE). One additional slide of the same tumor is stained with H&E.
2) A pathologist reviews the H&E slide and indicates the area to be collected for the CancerTYPE ID™ assay. The slides are sent to Aviara DX for analysis.
3) The test report from Aviara DX indicates the top 5 most probable sites of origin as determined from a k-nearest neighbor analysis and a prediction is derived. As a non-limiting example, if the prediction for the patient is the lung as the tumor of unknown origin, the patient's tumor cells can be assessed for pathway activation.
4) Tumor cells (e.g., CTCs) are isolated from blood and prepared for analysis as described, e.g., in Example 1 of PCT Publication No. WO 2011/008990, which is herein incorporated by reference in its entirety for all purposes. Alternatively, a fine needle biopsy can be used to prepare a tumor cell extract as described, for example, in Example 2 of PCT Publication No. WO 2011/008990, which is herein incorporated by reference in its entirety for all purposes. The cell preparations are assayed as described in either Example 1 or Example 2 herein. The activation profile is evaluated in a similar manner as described in Example 4 or Example 5 herein. The appropriate targeted therapy or combination of targeted therapies is then selected.

### Example 7

### Data Analysis for Quantitation of Signal Transduction Pathway Proteins in Cancer Cells

This example illustrates the quantitation of the expression and/or activation levels of one or more analytes such as one or more signal transduction proteins in a biological sample (e.g., blood or tumor tissue) against a standard curve generated for the particular analyte of interest.

In some embodiments, each CEER slide is scanned at three photomultiplier (PMT) gain settings to improve sensitivity and reduce the impact of saturation. Perkin Elmer ScanArray Express software is used for spot finding and signal quantitation. The identifiers for each spot are imported from a GenePix Array List (.gal) file. The de-identified study specific number for each clinical sample on a slide is incorporated into the resulting data set.

In other embodiments, background corrected signal intensities are averaged for replicate spots printed in triplicate. The relative fluorescence value of the respective reagent blank is subtracted from each sample. Several quality criteria are used to filter data from further analysis including limits on the spot footprint, coefficient of variation for spot replicates, overall pad background and the intensity of the reagent blank.

For each assay, a sigmoidal standard curve can be generated from multiple (e.g., two, three, four, five, six, seven, etc.) concentrations of serially diluted cell lysates prepared from cell lines such as MD-468 (HER1 positive), SKBr3 (HER2 positive), BT474 (HER2 and p95HER2 positive), HCC827 (c-MET and HER1 positive), T47D stimulated with IGF (IGF1R positive), and/or T47D stimulated with HRG (HER3 positive). Each curve can be plotted as a function of signal intensity vs. log concentration derived units, CU (Computed Unit). The data can be fit to a five parameter equation (5PL) by nonlinear regression (Ritz, C. and Streibig, J. C., J. Statistical Software, 12, 1-22 (2005)), simultaneously fitting all three dilutions of the capture antibody. Fitting is carried out using R, an open source statistical software package (Development Core Team, R: A language and environment for statistical computing. R Foundation for Statistical Computing, Vienna, Austria. ISBN 3-900051-07-0, URL http://www.R-project.org.R (2008)). To avoid over parameterization of the mathematical model and thereby improve accuracy, four parameters can be constrained, while each dilution can be solved for an individual inflection point. This process can be repeated for each PMT gain setting of 45, 50 and 60. This results in nine standard curves generated per assay, from three dilutions of capture antibody and three PMT scans. The built-in redundancy in the assay allows for one or more of the dilution/scan combinations to be eliminated if the fit of the standard curve has an R² less than 0.95 and thus improves subsequent predictions.

CU Calculation (Based on Standard Curve)

The individual predictions from each of the standard curves (e.g., 3 capture antibody dilutions and 3 PMT gain-set scanning) can be combined into a single, final prediction. For each prediction, the slope of the point on the standard curve is calculated. This slope is taken with log-units on the x-axis, i.e., the units in the denominator of the slope are log Computed Units (CU). Second, a weighted average of the predictions is calculated, where the weights are determined from the slopes. Specifically, the weights are summed, and each point is given a weight equal to its slope divided by the total slopes. Each assay can be validated against predictions for known controls.

### Example 8

### Detection and Quantification of Total Oncogenic Protein and Activated Protein Levels in Caucasian Patient Samples of NSCLC

This example demonstrates a method for the detection and quantification of protein levels in malignant tumor tissues biopsied from Caucasian patients presenting with non-small cell lung cancer (NSCLC). In one particular embodiment, the present method enables the detection and measurement of expression levels of a plurality of biomarkers associated with NSCLC, such as but not limited to cMET, HER1, HER2, HER3, PI3K, SHC and CK. The method can also detect and quantify the levels of total and activated (e.g., phosphorylated) protein in the same biological sample harvested from a patient's lung tumor tissue.

In certain embodiments, protein levels are determined along with activated protein levels using a multiplexed protein microarray platform array such as CEER. The expression levels of multiple full-length and modified (i.e., truncated, phosphorylated, and/or methylated) proteins can be detected and quantified. The expression levels of the protein can be quantified by comparison to control proteins, such as but not limited to IgG and CK. In certain embodiments, co-expression levels of a plurality of oncogenic proteins, such as ligands, receptors, and downstream signaling effectors, can be determined and directly compared.

In certain embodiments, the biological samples used in the present invention can be isolated from tumor tissue samples collected from patients with NSCLC. The lung tumor tissue can carry activating genetic mutations associated with tyrosine kinase inhibitor resistance. In NSCLC, KRAS mutations (e.g., G12C, G12D, G12R and/or G12V) are associated with primary resistance to EGFR TKIs and the EGFR mutation T790M is mainly associated with secondary or acquired resistance.

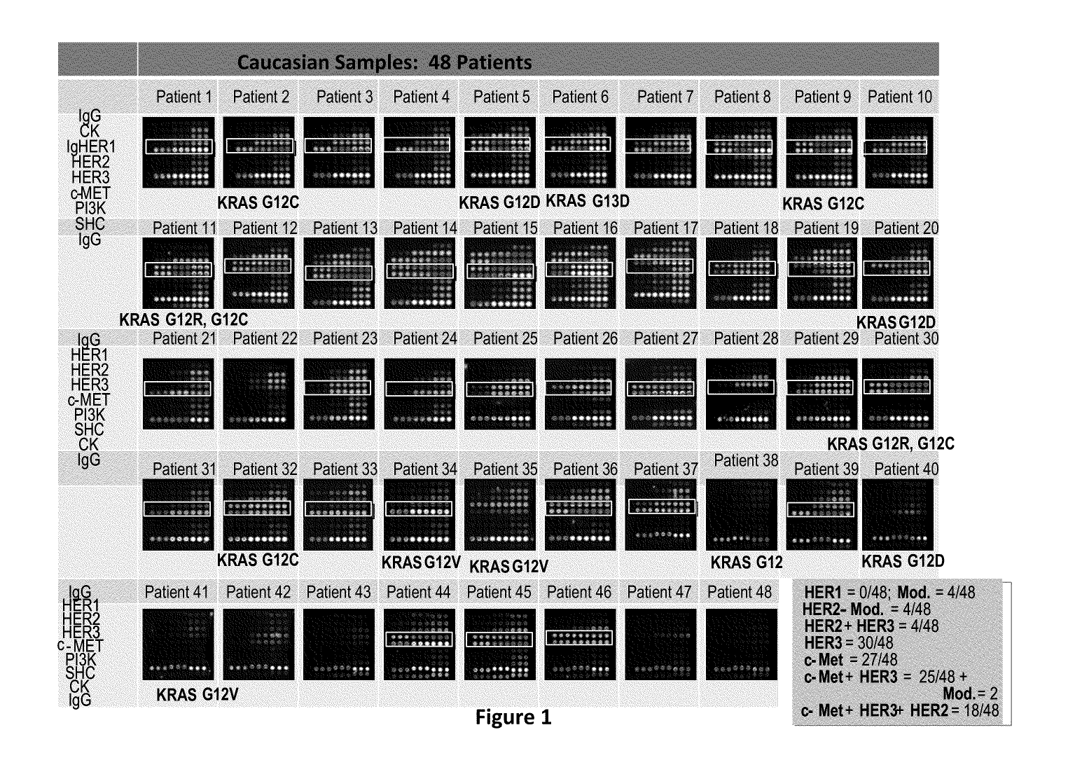

FIG. 1 illustrates that levels of HER1 and HER2 proteins were very low in most of the NSCLC tumor samples from Caucasian patients. In contrast, HER3 and cMET proteins were expressed at medium to high levels in more than 50% of the patient samples. In FIG. 1, the total levels of HER1, HER2, HER3, cMET, CK, PI3K, and SHC proteins are clearly visualized on the array. FIG. 1 also demonstrates that the method described herein can be used to detect protein expression in NSCLC samples with KRAS mutations, such as but not limited to G12C, G12D, G12V, or the homozygous mutation of G12R, G12C. FIGS. 2C, 2D, 3C, and 3D provide more illustrations of the protein expression profile detected in the NSCLC tumor samples. In particular, these figures demonstrate that expression levels for HER1, HER2, HER3 and cMET can be grouped into three distinct categories of high, medium, and low levels of expression. FIG. 4B provides a summary chart of the expression levels of HER1, HER2, HER3, cMET and CK in the tumor tissue collected from 51 Caucasian patients with NSCLC. FIG. 5 illustrates that protein levels of a plurality of proteins can be compared to determine relationships between oncogenic signaling molecules. FIG. 5 shows that high cMET expression was associated with medium to high HER3 expression in the NSCLC samples from Caucasian patients. FIG. 5 also illustrates that Caucasian patients tend to have high cMet levels as compared to Asian patients. FIG. 6 shows an example of two patients who expressed high levels of total cMET and HER3 proteins who also expressed phosphorylated cMET, HER3, and PI3K.

### Example 9

### Detection and Quantification of Total Oncogenic Protein and Activated Protein Levels in Asian Patient Samples

This example demonstrates a method for the simultaneous detection and quantification of oncogenic protein levels in malignant tumor tissues biopsied from Asian patients presenting with non-small cell lung cancer (NSCLC). In one particular embodiment, the present method enables the detection and measurement of expression levels of a plurality of oncogenic proteins (e.g., cMET, HER1, HER2, HER3, PI3K, SHC, and CK) as well as expression levels of their activated (e.g., phosphorylated) forms in a biological sample harvested from patient tumor tissue.

In certain embodiments, total and activated protein levels are determined using COPIA (also referred to as CEER) arrays. The expression levels of multiple full-length and modified (i.e., truncated, phosphorylated, and/or methylated) proteins can be detected and quantified. The expression levels of the total protein can be quantified by comparison to a control protein such as, but not limited to IgG. In certain embodiments, co-expression levels of a plurality of oncogenic proteins, such as ligand, receptors, and downstream signaling effectors, can be measured and directly compared among biological samples.

In certain embodiments, the biological samples used in the present invention can be isolated from patients with NSCLC. Lung tumor samples harvested from these patients can contain activating genetic mutations associated with tyrosine kinase inhibitor resistance. In NSCLC, KRAS mutations are associated with primary resistance to EGFR TKIs and the EGFR mutation T790M is mainly associated with secondary or acquired resistance.

In certain embodiments, the dynamic range of protein expression detected using the CEER assay can be categorized into three separate groups based on the Relative Fluorescence Unit (RFU) values. The ranges of HER1 expression were established to be 1-10,000 RFU for low levels of expression; 10,000-36,000 for medium; and 36,000 and above for high. An expression of HER2 below 50,000 RFU is defined as low, and above 50,000 is set as medium expression. For HER3, below 5,000 RFUs is considered low expression; 5,000-30,000 RFU is medium; and over 30,000 is high. In certain embodiments, the low level of cMET is at less than 10,000 RFU; medium from 10,000-40,000; and high at over 40,000 RFU.

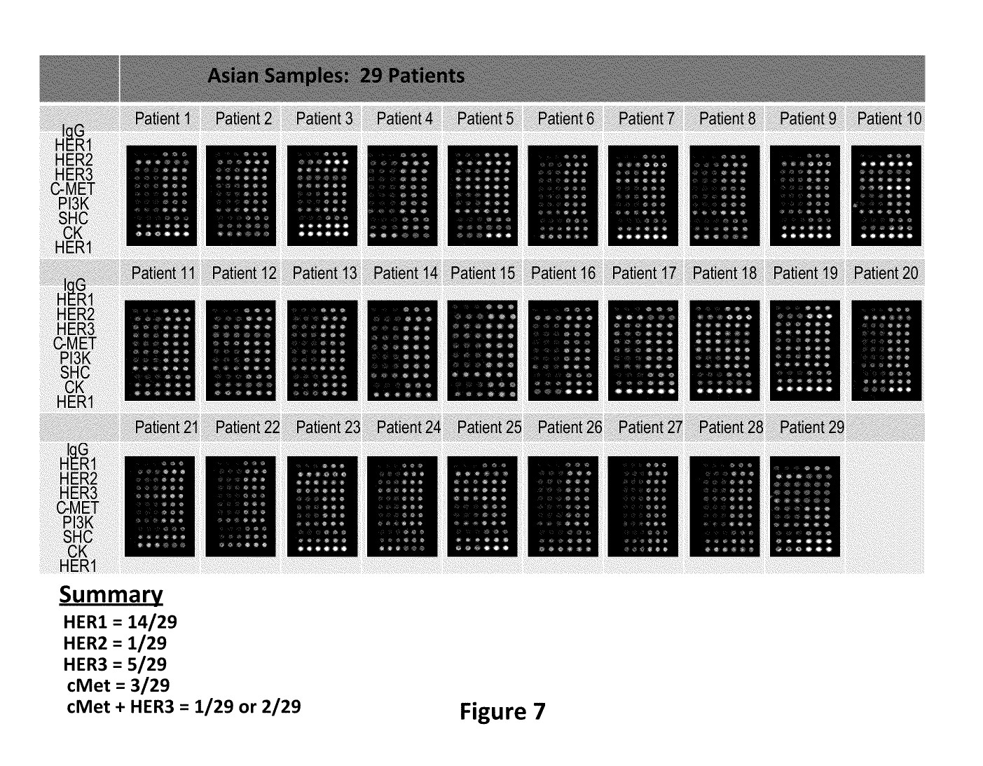

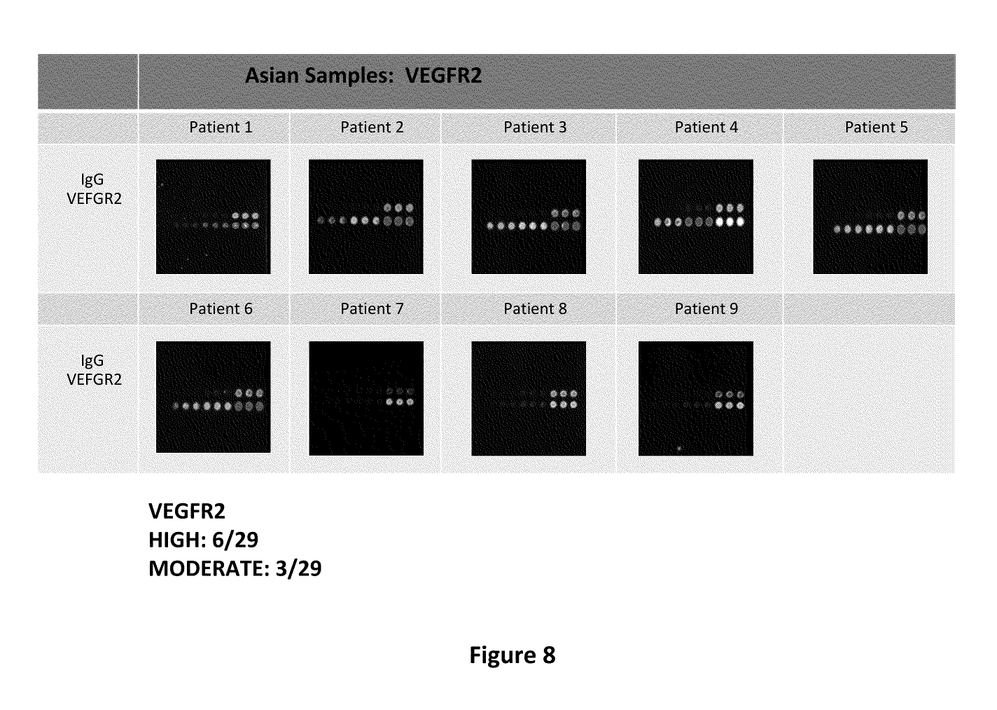

FIG. 7 illustrates the dynamic range of expression levels of HER1, HER2, HER3, cMET, PI3K, SHC, and CK in 29 Asian patient samples with NSCLC. In particular, about 48% of the patients expressed high levels of HER1, and most expressed low levels of HER2, HER3 and cMET. A very low percentage of patients sampled co-expressed high levels of cMET and HER3 (1 out of 29 patients). This is in contrast to a study performed in Caucasian patients, wherein 27 out of 51 NSCLC tumor samples co-expressed high levels of cMET and medium to high levels of HER3. FIGS. 2A, 2B, 3A and 3B provide more illustration of the HER1, HER2, HER3 and cMET expression profiles in the tissue samples from Asian patients with NSCLC. The expression levels can be grouped into three distinct categories based on the extended RFU values from the CEER assay. FIG. 4A provides a summary chart of the expression profile of HER1, HER2, HER3, cMET, and CK. FIG. 5 shows that low expression of cMET was most prevalent in the cohort of Asian patient samples. FIG. 5 also shows that 4 Asian patients with NSCLC expressed medium levels of cMET and high levels of HER3. FIG. 8 illustrates that the NSCLC tumor samples harvested from Asian patients exhibited variable levels of expression of VEGFR2. In particular, 6 of the 29 patient samples exhibited high levels of VEGR2 and 3 patients displayed moderate levels.

### Example 10

### Detection of Activated Levels of cMet, HER1, HER2, HER3, IGF-1R, c-Kit, PI3K, Shc, and CK in Various Cancer Cell Lines

This example demonstrates the detection of activated (phosphorylated) levels of cMET, HER1, HER2, HER3, IGF-1R, c-Kit, PI3K, SHC and CK proteins in cancer cell lines following cMET stimulation or inhibition. In some embodiments, the presence and/or activation state of oncogenic proteins correlated to primary and/or secondary resistance to EGFR TKI therapy can be measured using a proximity assay such as CEER.

In one particular embodiment, cells from a human cancer cell line (e.g., the NCI-N87 cell line established from a liver metastasis of a gastric carcinoma from patient; Park et al., Cancer Res., 50:2773-2780, 1990) were treated with different concentrations of cMET inhibitor. The levels of expression and activation of proteins associated with cancer were detected in the cells using the highly sensitive CEER array. Dose response curves were used to calculate EC50 values which are indicative of the inhibition and activation states of the analyzed proteins.

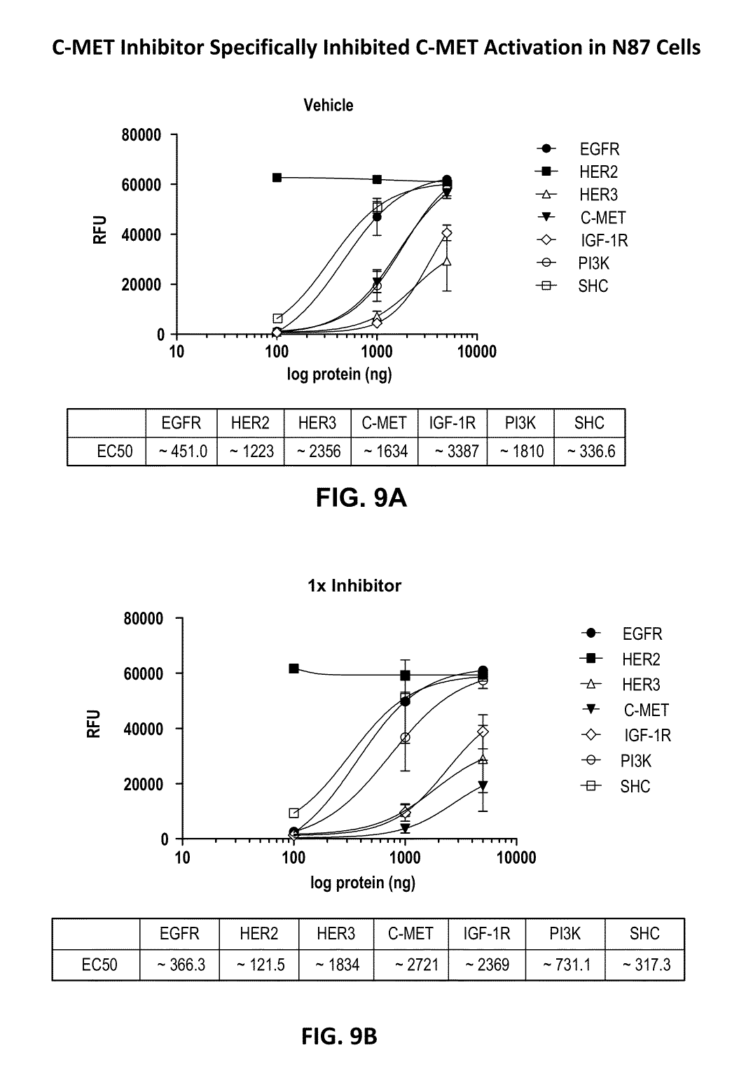

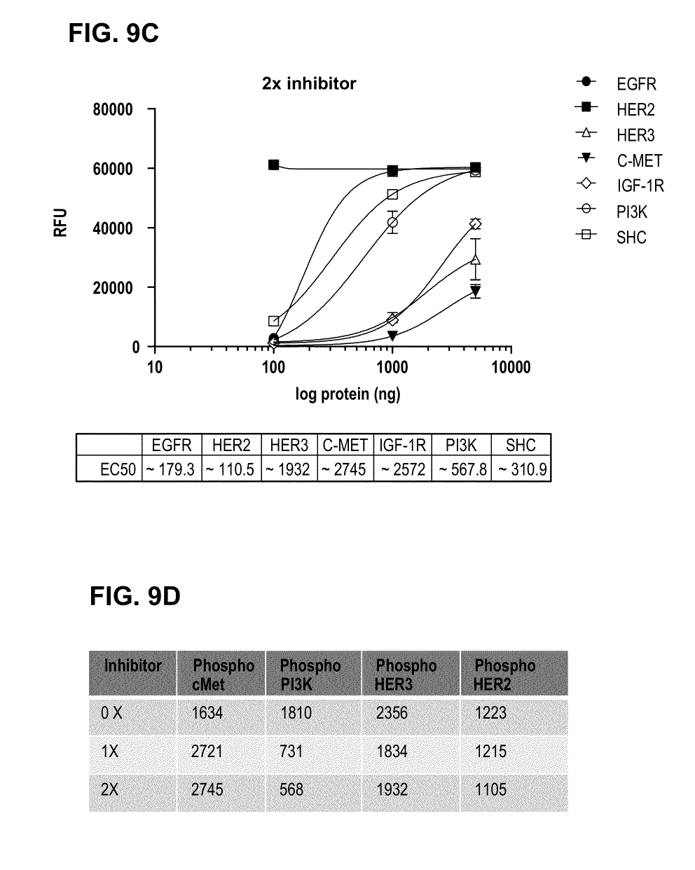

FIG. 9 illustrates that cMET inhibitor specifically inhibited cMET activation in N87 cells. Comparisons of the drug response curves in FIGS. 9A, 9B and 9C as well as the summary chart in FIG. 9D show that increased inhibitor concentration caused an increase in the EC50 value of phospho-cMET, which demonstrates cMET inhibition. Exposing the cells to 1× concentration of cMET inhibitor dramatically induced PI3K activation, as noted by the sharp decrease in EC50. FIG. 9D also shows slight activation of HER3 in the presence of cMET inhibitor. Increasing the concentration of cMET inhibitor to 2× induced a further increase in PI3K activation, suggesting that phosphorylated PI3K in the experiment was due to a cMET-independent signaling pathway.

In one particular embodiment, cells from a NSCLC cell line (e.g., HCC827 cells or other gefitinib sensitive lung adenocarcinoma cells with EGFR-activating mutations; American Type Culture Collection, Manassas, Va.) were treated with different concentrations of ligand or signaling molecule (e.g., the ligand for the cMET receptor, HGF). The levels of expression and activation of proteins associated with cancer were measured in the treated cells with high sensitivity using CEER arrays. By varying the amount of ligand exposed to the cancer cells, the expression profiles of the analyzed proteins were correlated to the amount of ligand used to stimulate the cells. Dose response curves were used to calculate EC50 values which can be used to interpret the inhibition and activation states of the analyzed proteins.

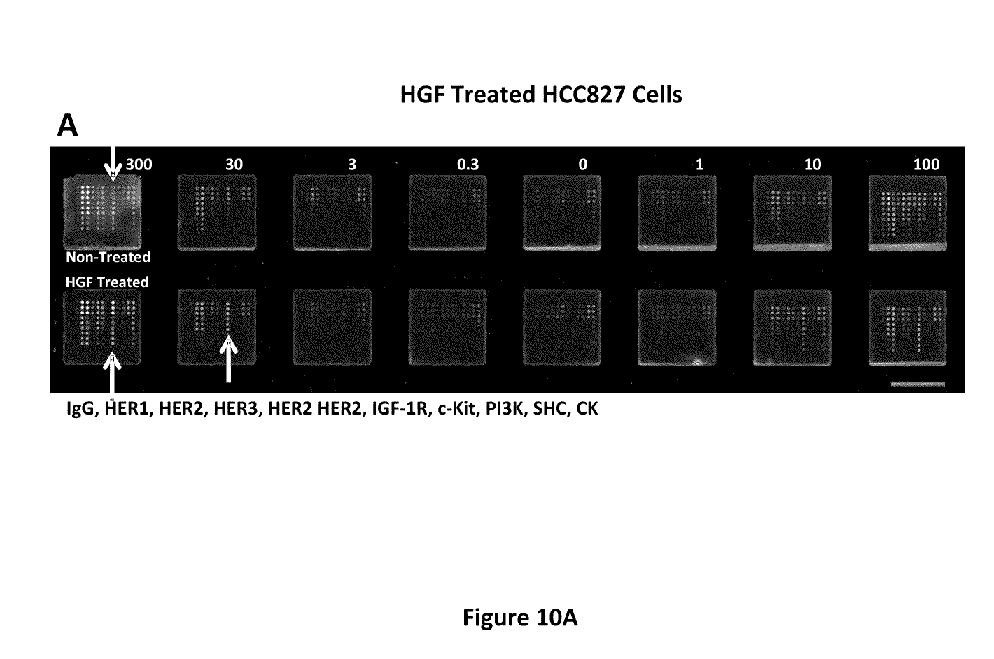

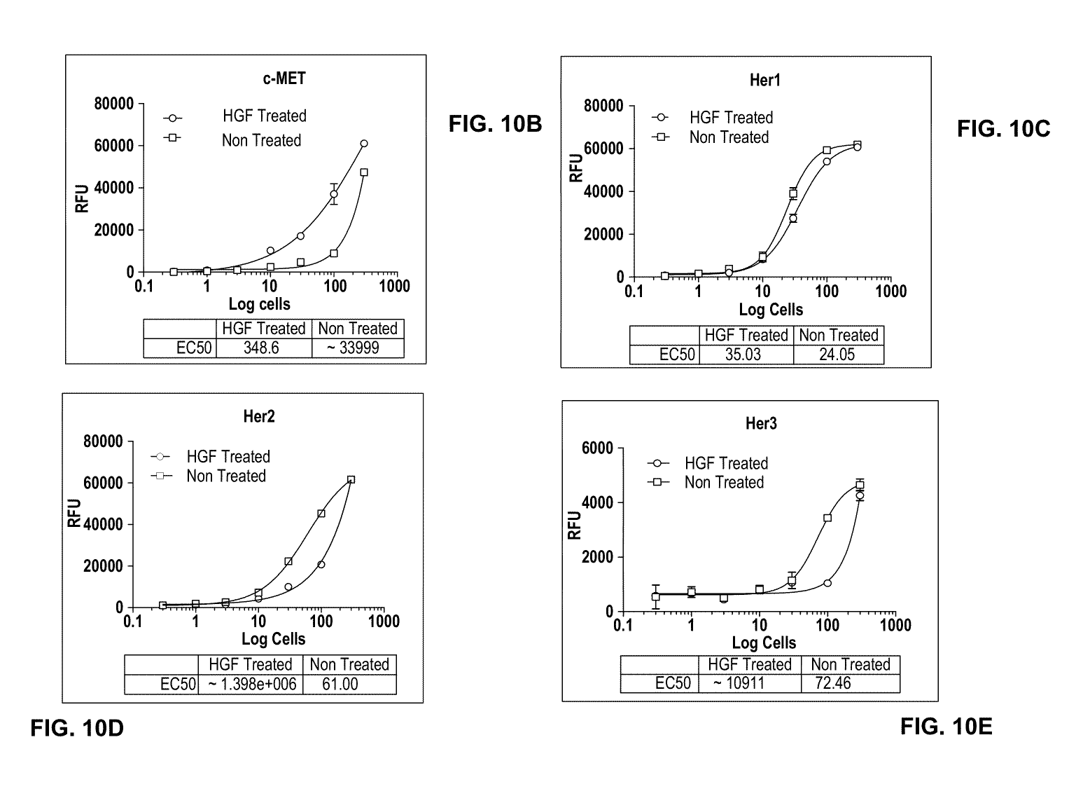

FIG. 10 illustrates the difference in protein expression in HCC827 cells following HGF stimulation. FIG. 10A shows that the expression levels of activated cMET, HER1, HER2, HER3, IGF-1R, c-Kit, PI3K, SHC, and CK proteins responded to varying HGF levels. FIG. 10B illustrates that HGF activated cMET effectively, while on the other hand, HGF decreased HER1, HER2, and HER3 activation, as determined by the increased EC50 value following HGF treatment. FIG. 10E illustrates that HGF and cMET binding interferes with HER3 activation, and HER1 and HER2 to a lesser extent (see also, FIGS. 10C and 10D).

This example demonstrates that cMET interacts with HER1, HER2, and HER3. Although the interaction is weak, it is sufficient to transphosphorylate and activate HER1, HER2, and HER3. When cMET binds to HGF, the complex forms stable homodimers which do no interact with HER1, HER2 and HER3. This results in decrease phosphorylated HER1, HER2 and HER3 upon HGF stimulation.

### Example 11

### Selection of Anticancer Drug Therapy for a Patient with a Malignancy Involving Aberrant cMet Signaling

This example demonstrates a method of determining the selection of an appropriate therapy for a subject with a malignancy involving aberrant cMet signaling. The method is based upon the expression/activation profiling of analytes of signaling transduction pathway proteins (e.g., HER1, HER2, HER3, cMet, IGF1R, cKit, PI3K, Shc, VEGFR1, VEGFR2, VEGFR3, truncated cMet and/or truncated HER3) in the subject's tumor tissue sample using, e.g., the Collaborative Enzyme Enhanced Reactive Immunoassay (CEER) as described herein. In addition, the expression/activation profiling of kinases and other signal transduction pathway components on a serial sampling of tumor tissues from a subject provides valuable information on changes occurring in tumor cells as a function of time and therapeutic regimens. This temporal profiling of tumor progression enables clinicians to monitor rapidly evolving cancer signatures in each subject. This example illustrates the use of the method of the present invention to predict a subject's response to therapies based on the expression/activation profile of a plurality of signaling pathway components in a subject's tumor tissue sample.

As a non-limiting example, a patient with a malignancy involving aberrant cMet signaling (such as, but not limited to, carcinomas of the breast, liver, lung, gastric, ovary, kidney, and thyroid, and non-small cell lung cancer (NSCLC)) will likely respond to a cMet inhibitor due to expression of HER3, HGF/SF and cMet transcripts and proteins. Examples of cMet inhibitors include, but are not limited to, monoclonal antibodies, such as AMG102 and MetMAb; small molecule inhibitors of cMet, such as ARQ197, JNJ-38877605, PF-04217903, SGX523, GSK 1363089/XL880, XL184, MGCD265, and MK-2461, and combinations thereof. In another aspect, a patient with a malignancy involving aberrant cMet signaling such as NSCLC with a KRAS mutation will likely respond to a cMet inhibitor due to expression of HER3, HGF/SF and cMet. In yet another aspect of the present invention, a patient with a malignancy involving aberrant cMet signaling such as NSCLC and expression of activated PI3K will likely respond to a cMet inhibitor due to the expression of cMet and HGF/SF. In another non-limiting example, a patient with a malignancy involving aberrant cMet signaling such as NSCLC will likely respond to a cMet inhibitor due to activation of cMet (e.g., phospho-cMet) or co-activation of cMet and HER3 (e.g., phospho-cMet and phospho-HER3). In another aspect, a patient with a malignancy involving aberrant cMet signaling such as NSCLC will likely respond to a cMet inhibitor due to the expression of truncated cMet protein and high expression of HER3. In yet another aspect, a patient with a malignancy involving aberrant cMet signaling such as NSCLC will likely respond to a cMet inhibitor due to the expression of truncated HER3 protein and high expression of cMet. In another non-limiting example, a patient with a malignancy involving aberrant cMet signaling such as NSCLC will likely respond to a cMet inhibitor due to cMet expression and activation. In an additional non-limiting example, a patient with a malignancy involving aberrant cMet signaling such as NSCLC will likely respond to a cMet inhibitor due to the expression and activation of cMet and HER3, along with low expression of HER1 and HER2.

This example demonstrates the determination of whether to administer a cMet inhibitor alone or a cMet inhibitor in combination with a pathway-directed therapy for a subject with a malignancy involving aberrant cMet signaling, based upon the differences between the expression of analytes of signaling transduction pathway proteins (e.g., HER1, HER2, HER3, cMet, IGF1R, cKit, PI3K, Shc, VEGFR1, VEGFR2, VEGFR3, truncated cMet and/or truncated HER3) in the subject's tumor tissue sample using the Collaborative Enzyme Enhanced Reactive Immunoassay (CEER) as described herein.

A pathway-directed therapy includes the use of therapeutic agents for the treatment of a patient's disease wherein the agents can alter the expression level and/or activated level of one or more signaling pathway components. Non-limiting examples of pathway-directed therapies include cMet inhibitors, EGFR inhibitors, VEGFR inhibitors, pan-HER inhibitors, and combinations thereof. Examples of cMet inhibitors include, but are not limited to, neutralizing antibodies such as MAG102 (Amgen) and MetMab (Roche), and tyrosine kinase inhibitors (TKIs) such as ARQ 197, XL 184, PF-02341066, GSK1363089/XL880, MP470, MGCD265, SGX523, PF04217903, JNJ38877605, and combinations thereof. Examples of EGFR inhibitors include, but are not limited to, Cetaximab, Panitumumab, Matuzumab, Nimotuzumab, ErbB1 vaccine, Erlotinib, Gefitinib, EKB 569, CL-387-785, and combinations thereof. Examples of VEGF inhibitors include, but are not limited to, Bevacizumab (Avastin), HuMV833, VEGF-Trap, AZD 2171, AMG-706, Sunitinib (SU11248), Sorafenib (BAY43-9006), AE-941 (Neovastat), Vatalanib (PTK787/ZK222584), and combinations thereof. Non-limiting examples of pan-HER include PF-00299804, neratinib (HKI-272), AC480 (BMS-599626), BMS-690154, PF-02341066, HM781-36B, CI-1033, BIBW-2992, and combinations thereof.

As a non-limiting example, a patient with a malignancy involving aberrant cMet signaling (such as, but not limited to, carcinomas of the breast, liver, lung, gastric, ovary, kidney, and thyroid, and non-small cell lung cancer) will likely respond to and should receive a therapy comprising a cMet inhibitor in combination with a pathway-directed therapy due to the co-activation of EGFR, HER2, and HER3, along with cMet expression. In one particular instance, a patient with a malignancy involving aberrant cMet signaling such as NSCLC will likely respond to a combination therapy comprising a cMet inhibitor and an EGFR inhibitor due to expression and activation of cMet, EGFR and HER2. On the other hand, a patient will likely be resistant to a combination therapy comprising a cMet inhibitor and an EGFR inhibitor if co-activation of HER2 and HER3 is determined in the patient's tumor sample. In another aspect, a patient with a malignancy involving aberrant cMet signaling such as NSCLC will likely respond to a cMet inhibitor in combination with a pathway-directed therapy due to co-expression of cMet, HER2 and HER3, in addition to activation of PI3K. In another particular instance, a patient with a malignancy involving aberrant cMet signaling such as NSCLC will likely respond to and should receive a combination therapy comprising a cMet inhibitor and a VEGF inhibitor due to the expression and activation of cMet and HER3, along with the expression and activation of VEGFR1, VEGFR2 and/or VEGFR3. In another non-limiting example, a patient with a malignancy involving aberrant cMet signaling such as NSCLC with an EGFR mutation and expression of cMet will likely respond to a combination therapy of a cMet inhibitor and a pathway-directed therapy.

In a non-limiting example, a patient with a malignancy involving aberrant cMet signaling (such as, but not limited to, carcinomas of the breast, liver, lung, gastric, ovary, kidney, and thyroid, and non-small cell lung cancer) will likely respond to a cMet inhibitor in combination with Iressa (i.e., gefitinib) and a VEGFR inhibitor due to co-activation of cMet, VEGFR2 (see, e.g., FIG. 8), and EGFR. In another non-limiting example, a patient with a malignancy involving aberrant cMet signaling such as NSCLC will likely respond to a combination therapy with a cMet inhibitor, a VEGFR inhibitor, and Tykerb (i.e., lapatinib) due to co-activation of cMet, VEGFR2, EGFR, HER2, HER3, and truncated HER2 protein (e.g., p95HER2).

### Example 12

### Selection of Targeted Agent(s) Based on Matching Differential Protein Expression and Mutational Profile in NSCLC Patients

This example provides a follow-on study that further demonstrates a method for the detection and quantification of protein levels in malignant tumor tissues biopsied from Asian and Caucasian patients with non-small cell lung cancer (NSCLC). In one embodiment, the present method enables the detection and measurement of expression and/or activation levels of a plurality of biomarkers associated with NSCLC, including, but not limited to, EGFR, ErbB2, ErbB3, cMET, IGF1R, cKIT, Shc, PI3K, AKT, ERK, VEGFR2, Src, FAK, Stat5, JAK2, and CrKL. The method can also detect and quantify the levels of total and activated (e.g., phosphorylated) protein in the same biological sample harvested from a patient's lung tumor tissue.

### BACKGROUND

Treatment of non-small cell lung cancer (NSCLC) with tyrosine kinase inhibitors (TKIs) shows initial response, but most tumors develop acquired resistance to TKIs due to secondary resistance mutations (e.g., T790M) and amplification of other RTKs. Patients stratified for mono therapy treatment based on genotype alone is not sufficient as other kinases contribute to survival of the tumor cells, and thus necessitating inhibition of multiple kinases.

### Methods:

The Collaborative Enzyme Enhanced Reactive-immunoassay (CEER) is a multiplexed protein microarray platform requiring co-localization of two detector enzyme-conjugated-antibodies. CEER is capable of high detection sensitivity from viable tissue samples such as a fine needle aspirate (FNA) and circulating tumor cells (CTC). In some instances the sample is collected using a 23-35 gauge needle. Lysates were prepared from frozen tissues obtained surgically from 71 Caucasian and 29 Asian patients with NSCLC. Expression and activation of EGFR, ErbB2, ErbB3, cMET, IGF1R, cKIT, Shc, PI3K, AKT, ERK, VEGFR2, Src, FAK, Stat5, JAK2, CrKL, and other pathway proteins were profiled. Genotyping on panel of mutations in KRAS, EGFR, and BRAF were performed on all samples.

### Results:

We have observed differential expression patterns between Asian and Caucasian patients. KRAS was mutated in 30% of all the patients. In Asian patients, EGFR over-expression was higher (27%) compared to the Caucasians (3%). Caucasian patients expressed high HER3 (58%), cMET (25%), and co-expression of both cMET and HER3 (27%). VEGFR2 was highly expressed in 17% of all patients. Patients over-expressing cMET, HER3, and HER2 will benefit from a combination of pan-HER and cMET inhibitors. Those over-expressing cMET and EGFR will benefit from cMET and EGFR inhibitors. Lastly, patients over-expressing VEGFR2 and cMET will benefit from cMET and VEGFR inhibitors. The methods described herein can provide a comprehensive differential biomarker prevalence analysis for each NSCLC sub-populations for all tested RTKs and signaling proteins.

### CONCLUSION

Mono therapy and patient selection based on genotype is not an effective criteria for treatment options. Instead, a comprehensive pathway profile using FNA/CTC should guide selection of appropriate therapy (e.g., combined or sequenced), and shifts in pathway profile should be monitored for appropriate response.

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1. A method for therapy selection for a subject with a malignancy involving aberrant c-Met signaling, said method comprising:
(a) detecting and/or quantifying the expression level and/or activation level of cMet protein in a sample taken from the subject;
(b) detecting and/or quantifying the expression level and/or activation level of HER3 protein in the sample;
(c) comparing the expression level and/or activation level of cMet protein and/or HER3 protein in the sample to (i) the expression level and/or activation level of a control protein and/or (ii) the expression level and/or activation level of cMet protein and/or HER3 protein in a control sample; and
(d) determining whether to administer a cMet inhibitor alone or a cMet inhibitor in combination with a pathway-directed therapy based upon a difference between the expression level and/or activation level of cMet protein and/or HER3 protein in the sample compared to the control protein and/or control sample.

2. The method of claim 1, wherein the control protein comprises IgG.

3. The method of claim 1, wherein the control sample comprises a cell line or tissue sample not having said malignancy involving aberrant c-Met signaling.

4. (canceled)

5. (canceled)

6. (canceled)

7. (canceled)

8. The method of claim 1, wherein said malignancy involving aberrant c-Met signaling is non-small-cell lung cancer (NSCLC).

9. The method of claim 1, wherein step (d) comprises determining that the cMet inhibitor should be administered alone when the expression level and/or activation level of cMet protein in said sample is determined to range from medium to high compared to the control protein and/or control sample.

10. (canceled)

11. (canceled)

12. The method of claim 1, wherein step (d) comprises determining that the cMet inhibitor should be administered alone when the expression level and/or activation level of HER3 protein in said sample is determined to range from medium to high compared to the control protein and/or control sample.

13. (canceled)

14. The method of claim 1, further comprising detecting and/or quantifying the expression level of HER1 protein, HER2 protein, HGF/SF protein, or combinations thereof.

15. The method of claim 14, wherein step (d) further determining that the cMet inhibitor should be administered alone when the expression level of HER1 protein and the expression level HER2 protein in said sample is low compared to the control protein and/or control sample.

16. The method of claim 14, wherein step (d) comprises determining that the cMet inhibitor should be administered alone when the expression level of HGF/SF protein in said sample is determined to range from medium to high compared to the control protein and/or control sample.

17. The method of claim 1, wherein said subject has a KRAS mutation.

18. (canceled)

19. The method of claim 17, wherein step (d) comprises determining that the cMet inhibitor should be administered alone when the KRAS mutation is present in said sample and the expression level and/or activation level of cMet protein, HER3 protein, and HGF/SF protein in said sample is each independently determined to range from medium to high compared to the control protein and/or control sample.

20. The method of claim 1, wherein step (d) comprises determining that the cMet inhibitor should be administered alone when the expression level of cMet protein in said sample is determined to range from low to medium compared to the control protein and/or control sample, and when the activation level of cMet protein is high compared to the control protein and/or control sample.

21. The method of claim 1, wherein step (d) comprises determining that the cMet inhibitor should be administered alone when the expression level of HER3 protein in said sample is determined to range from low to medium compared to the control protein and/or control sample, and when the activation level of HER3 protein is high compared to the control protein or control sample.

22. The method of claim 1, further comprising detecting and/or quantifying the expression level and/or activation level of a truncated cMet protein in the sample.

23. The method of claim 22, wherein step (d) comprises determining that the cMet inhibitor should be administered alone when the expression level of the truncated cMet protein in the sample is detectable and the expression level of HER3 protein in said sample is determined to range from medium to high compared to the control protein and/or control sample.

24. The method of claim 1, further comprising detecting and/or quantifying the expression level and/or activation level of a truncated HER3 protein in the sample.

25. The method of claim 24, wherein step (d) comprises determining that the cMet inhibitor should be administered alone when the expression level of the truncated HER3 protein in the sample is detectable and the expression level of cMet protein in said sample is determined to range from medium to high compared to the control protein and/or control sample.

26. The method of claim 1, further comprising detecting and/or quantifying the expression level and/or activation level of PI3K protein in the sample.

27. The method of claim 26, wherein step (d) comprises determining that the cMet inhibitor should be administered alone when PI3K protein is activated in said sample and the expression level and/or activation level of cMet protein and HGF/SF protein in said sample is each independently determined to range from medium to high compared to the control protein and/or control sample.

28. The method of claim 1, further comprising genotyping said subject for an EGFR mutation.

29. (canceled)

30. The method of claim 28, wherein step (d) comprises determining that the cMet inhibitor should be administered in combination with a pathway-directed therapy when said EGFR mutation is present and when the expression level of cMet protein in said sample is determined to range from medium to high compared to the control protein and/or control sample.

31. (canceled)

32. The method of claim 1, further comprising detecting and quantifying the expression level and/or activation level of EGFR protein, HER2 protein, PI3K protein, VEGFR1 protein, VEGFR2 protein, and/or VEGFR3 protein in the sample.

33. The method of claim 32, wherein step (d) comprises determining that the cMet inhibitor should be administered in combination with a pathway-directed therapy when the activation level of EGFR protein, HER2 protein, and HER3 protein in said sample is each independently determined to range from medium to high compared to the control protein and/or control sample and the expression level of cMet protein in said sample is determined to range from medium to high compared to the control protein and/or control sample.

34. The method of claim 32, wherein step (d) comprises determining that the cMet inhibitor should be administered in combination with a pathway-directed therapy when the activation level of PI3K protein in said sample is determined to range from medium to high compared to the control protein or control sample and the expression level of HER2 protein, HER3 protein, and cMet protein in said sample is each independently determined to range from medium to high compared to the control protein or control sample.

35. The method of claim 32, wherein step (d) comprises determining that the cMet inhibitor should be administered in combination with a pathway-directed therapy when the expression level and/or activation level of cMet protein, EGFR protein, and HER2 protein in said sample is each independently determined to range from medium to high compared to the control protein and/or control sample.

36. (canceled)

37. The method of claim 32, wherein step (d) comprises determining that the cMet inhibitor should be administered in combination with a pathway-directed therapy when the expression level and/or activation level of cMet protein, HER3 protein, and any one, two, or all three of VEGFR1-3 proteins in said sample is each independently determined to range from medium to high compared to the control protein and/or control sample.

38. (canceled)

39. (canceled)

40. (canceled)

41. A method for monitoring the status of a malignancy involving aberrant cMet signaling in a subject or monitoring how a patient with said malignancy is responding to a therapy, said method comprising:
(a) detecting and/or quantifying serial changes to the expression level and/or activation level of cMet protein in a sample taken from the subject;
(b) detecting and/or quantifying serial changes to the expression level and/or activation level of HER3 protein in the sample; and
(c) comparing the expression level and/or activation level of cMet protein and/or HER3 protein in the sample to (i) the expression level and/or activation level of a control protein over time and/or (ii) the expression level and/or activation level of cMet protein and/or HER3 protein in a control sample over time,
wherein an increasing expression level and/or activation level of cMet protein and/or HER3 protein over time indicates disease progression or a negative response to said therapy, and
wherein a decreasing expression level and/or activation level of cMet protein and/or HER3 protein over time indicates disease remission or a positive response to said therapy.

42-53. (canceled)
